(19) United States
(12) Patent Application Publication
(10) Pub. No.: US 2022/0087900 A1
(43) Pub. Date: Mar. 24, 2022

(54) POLYMER PROCESS BAGS AND METHODS FOR MANUFACTURING THE SAME

(71) Applicant: SIO2 MEDICAL PRODUCTS, INC., Auburn, AL (US)

(72) Inventors: Ahmad Taha, Auburn, AL (US); Michael Bucholtz, Balston Spa, NY (US); Robert S. Abrams, Auburn, AL (US)

(21) Appl. No.: 17/420,914

(22) PCT Filed: Jan. 7, 2020
(86) PCT No.: PCT/US2020/012638

§ 371 Date: Jul. 6, 2021

Related U.S. Application Data
(60) Provisional application No. 62/789,048, filed on Jan. 7, 2019.
(60) Provisional application No. 62/846,515, filed on May 10, 2019.
(60) Provisional application No. 62/864,416, filed on Jun. 20, 2019.
(60) Provisional application No. 62/876,800, filed on Jul. 22, 2019.

(51) Int. Cl. A61J 1/14; A61J 1/10; C12M 1/00; C23C 16/505; C23C 16/40; C23C 16/32; C23C 16/36; C23C 16/04; B23K 26/21

(52) U.S. Cl. CPC A61J 1/1468; A61J 1/10; C12M 23/14; C23C 16/505; B23K 26/21; C23C 16/325; C23C 16/36; C23C 16/045; C23C 16/401

(57) ABSTRACT

A pharmaceutical package comprises a polymeric wall having an interior surface and an outer surface; and a tie coating of SiOxCy and/or a barrier coating of SiOx and/or a protective coating of SiOxCy on the interior surface. The walls may be formed into the vessel by means of laser welding. The package can be, for example, a rigid container, a bioprocessing bag or a transfer bag, wherein the coatings afford improved barrier properties and/or are effective to block extractables/leachables from the substrate and any coatings thereon and the coatings are able to maintain their desirable characteristics described herein against stretching/elongation conditions. The bag or rigid container can be used in the entire process of the CAR T cell manufacture/therapy. A method of handling the coated packages comprises limiting stretching the packages. The method of monitoring or controlling the internal pressure of the coated packages is also described.

This application incorporates by reference in their entirety U.S. Provisional Application No. 62/789,048, filed Jan. 7, 2019; U.S. Provisional Application No. 62/846,515, filed May 10, 2019; U.S. Provisional Application No. 62/864,416, filed Jun. 20, 2019 and U.S. Provisional Application No. 62/876,800 filed Jul. 22, 2019.

### FIELD

The present disclosure relates to the technical field of coated surfaces, for example interior surfaces of pharmaceutical packages or other vessels such as polymer bags or flasks for storing or other contact with fluids. Examples of suitable fluids include foods or biologically active compounds or body fluids, for example blood. The present disclosure also relates to a pharmaceutical package or other vessels and to a method for coating an inner or interior surface of a pharmaceutical package or other vessel such as a bioprocessing or transfer bag or a bag used for CAR-T cell therapy including CAR-T cell manufacturing or treatment.

The present disclosure also relates to improved methods for processing and manufacturing pharmaceutical packages or other vessels, particularly single-use bioprocessing bags and/or aseptic transfer bags for the preparation, storage and transport of biopharmaceutical solutions, intermediates and final bulk products. Optionally the processing bag can be a bag used for CAR-T cell therapy including CAR-T cell manufacturing or treatment.

### BACKGROUND OF THE DISCLOSURE

One important consideration in manufacturing pharmaceutical packages or other vessels for storing or other contact with fluids, for example vials and pre-filled syringes, is that the contents of the pharmaceutical package or other vessel desirably will have a substantial shelf life. During this shelf life, it can be important to isolate the material filling the pharmaceutical package or other vessel from the vessel wall containing it, or from barrier coatings or layers or other functional layers applied to the pharmaceutical package or other vessel wall to avoid leaching material from the pharmaceutical package or other vessel wall, barrier coating or layer, or other functional layers into the prefilled contents or vice versa.

Since many of these pharmaceutical packages or other vessels are inexpensive and used in large quantities, for certain applications it will be useful to reliably obtain the necessary shelf life without increasing the manufacturing cost to a prohibitive level.

For decades, most parenteral therapeutics have been delivered to end users in Type I medical grade borosilicate glass vessels such as vials or pre-filled syringes. The relatively strong, impermeable and inert surface of borosilicate glass has performed adequately for most drug products. However, the recent advent of costly, complex and sensitive biologics as well as such advanced delivery systems as auto injectors has exposed the physical and chemical shortcomings of glass pharmaceutical packages or other vessels, including possible contamination from metals, flaking, delamination, and breakage, among other problems. Moreover, glass contains several components which can leach out during storage and cause damage to the stored material.

In more detail, borosilicate pharmaceutical packages or other vessels exhibit a number of drawbacks. Glass is manufactured from sand containing a heterogeneous mixture of many elements (silicon, oxygen, boron, aluminum, sodium, calcium) with trace levels of other alkali and earth metals. Type I borosilicate glass consists of approximately 76% SiO2, 10.5% B2O3, 5% Al2O3, 7% Na2O and 1.5% CaO and often contains trace metals such as iron, magnesium, zinc, copper and others. The heterogeneous nature of borosilicate glass creates a non-uniform surface chemistry at the molecular level.

Glass forming processes used to create glass vessels expose some portions of the vessels to temperatures as great as 1200° C. Under such high temperatures alkali ions migrate to the local surface and form oxides. The presence of ions extracted from borosilicate glass devices may be involved in degradation, aggregation and denaturation of some biologics. Many proteins and other biologics must be lyophilized (freeze dried), because they are not sufficiently stable in solution in glass vials or syringes.

As a result, some companies have turned to plastic pharmaceutical packages or other vessels, which provide tighter dimensional tolerances and less breakage than glass.

Although plastic is superior to glass with respect to breakage, dimensional tolerances and surface uniformity, its use for primary pharmaceutical packaging remains limited due to the following shortcomings:
Gas (oxygen) permeability: Plastic allows small molecule gases to permeate into (or out of) the device. The permeability of plastics to gases can be significantly greater than that of glass and, in many cases (as with oxygen-sensitive drugs such as epinephrine), plastics previously have been unacceptable for that reason.
Water vapor transmission: Plastics allow water vapor to pass through devices to a greater degree than glass. This can be detrimental to the shelf life of a solid (lyophilized) drug. Alternatively, a liquid product may lose water in an arid environment.
Leachables and extractables: Plastic pharmaceutical packages or other vessels contain organic compounds that can leach out or be extracted into the drug product. These compounds can contaminate the drug and/or negatively impact the drug's stability. Leachables are chemicals that migrate from single-use processing equipment into various components of the drug product during manufacturing. Extractables are chemical entities (organic and inorganic) that can be extracted from disposables using common laboratory solvents in controlled experiments. They represent the worst-case scenario and are used as a tool to predict the types of leachables that may be encountered during pharmaceutical production. So extractables are the “potentials” and leachables are the “actuals.” More chemicals are available to leach from single-use processing equipment manufactured from polymers than from other materials such as glass and metal.

Clearly, while plastic and glass pharmaceutical packages or other vessels each offer certain advantages in pharmaceutical primary packaging, neither is optimal for all drugs, biologics or other therapeutics. Thus, there can be a desire for plastic pharmaceutical packages or other vessels, in particular plastic syringes, with gas and solute barrier properties which approach the properties of glass. Moreover, there can be a need for plastic syringes with sufficient lubricity and/or passivation or protective properties and a lubricity and/or passivation layer or pH protective coating which can be compatible with the syringe contents. There also can be a need for glass vessels with surfaces that do not tend to delaminate or dissolve or leach constituents when in contact with the vessel contents.

The materials used to fabricate single-use processing equipment, such as bioprocess bags or transfer bags, for biopharmaceutical manufacturing are usually polymers, such as plastic or elastomers (rubber), rather than the traditional metal or glass. Polymers offer more versatility because they are light-weight, flexible, and much more durable than their traditional counterparts. Plastic and rubber are also disposable, so issues associated with cleaning and its validation can be avoided. Additives can also be incorporated into polymers to give them clarity rivaling that of glass or to add color that can be used to label or code various types of processing components.

Given all the positive attributes that polymers possess, there are also some negatives to consider when working with them in pharmaceutical applications. In the presence of heat, light, oxygen, and various external influences (such as sterilization), polymers can degrade over time if not properly stabilized. Degradation can manifest itself as cracking, discoloration, or surface blooming/exudation—and this can severely affect the mechanical properties of the polymers. Stabilizing additives are incorporated into many polymers to prevent this degradation. However, the resulting formulation is more complex than that of metal and glass, and it makes materials such as plastic and rubber much more prone to leaching unwanted chemicals into drug product formulations when they are used in applications such as manufacturing or packaging. While such materials typically have certain downsides, their benefits greatly outweigh their associated risks.

When a plastic resin is processed, it is often introduced into an extruder, where it is melted at high temperatures and mixed by a series of screws into a homogenous molten mixture. Additional heat and shear are encountered by the plastic when it is extruded and molded or shaped into a final product form, such as tubing or a bioprocessing bag. The degree of potential degradation depends on the nature of a polymer's chemical composition, the manner in which it is processed or molded, and the end use of the finished product. For example, the inherent stability of a polymer substrate will be influenced by its molecular structure, polymerization process, presence of residual catalysts, and finishing steps used in production. Processing conditions during extrusion (e.g., temperature, shear, and residence time in the extruder) can dramatically affect polymer degradation. End-use conditions that expose a polymer to excessive heat or light (such as outdoor applications or sterilization techniques used in medical practices) can foster premature failure of polymer products as well, leading to a loss of flexibility or strength. If left unchecked the results often can be total failure of the plastic component.

Polymer degradation can be controlled by the use of additives in the plastic or elastomer system. These are specialty chemicals that provide a desired effect to a polymer. The effect can be stabilization that allows a polymer to maintain its strength and flexibility or performance improvement that adds color or some special characteristic such as antistatic or antimicrobial properties. Additives known as plasticizers can affect the stress-strain relationship of a polymer (1). Polyvinylchloride (PVC) is used for home water pipes and is a very rigid material. With the addition of plasticizers, however, it becomes very flexible and can be used to make intravenous (IV) bags and inflatable devices. Stabilizers incorporated into plastic and rubber are constantly working to provide much-needed protection to the polymer substrate. This is a dynamic process that changes according to the external stress on the system.

The utility of polymers in disposable bioprocess equipment (and in all medical or pharmaceutical applications) far outweighs the risks associated with their use. The key is to manage those risks proactively. It is important to ensure that the correct polymer is chosen for a given bioprocessing application. Many different types of plastic and elastomers are commercially available, each with different physical and chemical properties. Special consideration should be given to the compatibility of their additives. For example, many different phenolic antioxidants are on the market, each with the same active site (the hindered phenol moiety). The feature that sets them apart from one another is the remainder of each molecule, which is what makes them soluble or compatible with a given polymer substrate. An antioxidant that is compatible with nylon might not be the best choice for use in polyolefins.

Ensuring compatibility often lessens the amount of leaching that can occur. It is also very prudent to select polymers and additives that are approved for use in food-contact applications. Such compounds have already undergone a fair amount of analytical and toxicological testing, so a good amount of information is often available for them. These materials are often important products for resin and additive manufacturers, so there is less likelihood of product discontinuation. They are also regulated by the FDA, so significant changes in their composition or manufacturing processes have to be reported to the agency and customers that purchase the materials. Thus, a basic change control process is in place.

Polymers offer many advantages as the primary materials used in manufacturing disposable bioprocess equipment. Plastic and rubber substrates are susceptible to degradation during extrusion, molding, and certain end-use applications, so they must be stabilized with additives. Because of their complex formulations, these polymers are more prone to leachables than are some of the traditional materials used in bioprocessing equipment, such as glass and metal. Managing risks associated with polymer use can be accomplished by proper material selection, implementation of the industry-recommended testing programs, and partnering with the vendors that manufacture and sell single-use bioprocessing equipment.

Even with appropriate systems and protocols including in situ and post-processing testing of the products in place, improved product offerings are needed for bioprocessing bags and transfer bags which further mitigate the risks associated with known polymer-based solutions.

### SUMMARY OF THE DISCLOSURE

Particular embodiments of the disclosure are set forth in the following numbered paragraphs:
1. A pharmaceutical package or vessel used for CAR-T cell therapy including CAR-T cell manufacturing or treatment, comprising:
   a polymeric wall having an interior surface and an outer surface;
   a tie coating or layer of SiOxCy, wherein x is from about 0.5 to about 2.4 and y is from about 0.6 to about 3, on the interior surface of the wall; and/or
   a barrier coating or layer of SiOx, wherein x is from 1.5 to 2.9, on the interior surface of the wall, or when present, the tie coating or layer of SiOxCy; and/or
   a passivation coating or layer or pH protective coating or layer of SiOxCy or SiNxCy, wherein x is from about 0.5 to about 2.4 and y is from about 0.6 to about 3, on the interior surface of the wall or, when present, on the innermost surface of the tie coating or layer or the barrier coating or layer; and/or
   a surface layer or coating of any of, or combination of, the following:
      silicon-based barrier coating system;
      amorphous carbon coating;
      fluorocarbon coating;
      direct fluorination;
      antiscratch/antistatic coating;
      antistatic coating;
      antistatic additive compound in polymer;
      oxygen scavenging additive compound in polymer;
      colorant additive compound in polymer;
      or antioxidation additive compound in polymer,
   on any of the interior surface of the wall or, when present, the inner surface of any of the other coatings or layers.
2. The pharmaceutical package or vessel of paragraph 1, wherein the package or vessel is flexible or stretchable.
3. The pharmaceutical package or vessel of paragraph 2, wherein the package or vessel is a bag, a bioprocess bag or a transfer bag.
4. The pharmaceutical package or vessel of paragraph 3, wherein the polymeric wall(s) is formed into the vessel or package by means of laser welding after the wall(s) have been coated with the tie coating or layer and/or the barrier coating or layer and/or the passivation coating or layer or pH protective coating or layer and/or the surface layer or coating.
5. The pharmaceutical package or vessel of paragraph 3, wherein the polymeric wall(s) are formed into the vessel or package by means of laser welding before the wall(s) have been coated with the tie coating and/or the barrier coating or layer and/or the passivation layer or coating or pH protective layer or coating and/or the surface layer or coating.
6. The pharmaceutical package or vessel of paragraph 3, wherein the laser welding uses a laser beam to melt the wall(s) in the joint area of the parts of the walls to be joined by delivering a controlled amount of energy to a precise location.
7. The pharmaceutical package or vessel of paragraph 6, wherein a heat input of the laser beam is controlled by adjusting the laser beam size and/or moving the laser beam.
8. The pharmaceutical package or vessel of paragraph 7, wherein the laser beam is delivered to the joint area through the upper “transparent” part and is absorbed by the lower absorbing part, which converts infra-red (IR) energy into heat.
9. The pharmaceutical package or vessel of paragraph 8, wherein the parts of the wall(s) to be joined are held together by clamping for heat transfer between the parts.
10. The pharmaceutical package or vessel of any of paragraphs 1-9, further comprising carbon black and/or other absorbers blended into the resin of the polymeric wall.
11. The pharmaceutical package or vessel of paragraph 3, wherein the laser welding is facilitated by one or more micron-scale laser beams.
12. The pharmaceutical package or vessel of paragraph 3, wherein the laser welding utilizes fiber-optic cable, scan head with mirrors coated for appropriate wave length, focusing optics, and programmable multi-axis servo stages for accurate and reproducible laser beam delivery.
13. The pharmaceutical package or vessel of paragraph 12, wherein the laser welding further comprises one or more servo motors to move and precisely position the laser beam.
14. The pharmaceutical package or vessel of paragraph 2, wherein the pharmaceutical package is a bioprocessing bag, or a transfer bag.
15. The pharmaceutical package or vessel of paragraph 2, wherein the coating(s) is able to maintain its desirable characteristics described herein against stretching/elongation conditions.
16. The pharmaceutical package or vessel of paragraph 1, wherein the package or vessel contains a rigid structure.
17. The pharmaceutical package or vessel of paragraph 16, wherein the rigid structure is a rigid support structure, a frame, or a rigid box.
18. The pharmaceutical package or vessel of paragraph 15, wherein the layer(s) or coating(s) and the surface thereunder are being stretched/elongated by 5%, optionally 10%, optionally 20%, optionally 30%, optionally 40%, optionally 50%, optionally 70%, optionally 90%, optionally 100%, optionally 150%, optionally 200% of the original size.
19. The pharmaceutical package or vessel of paragraph 15, wherein the layer(s) or coating(s) affords improved barrier properties to gases, moisture and solvents and maintains the blocking properties after being stretched/elongated.
20. The pharmaceutical package or vessel of paragraph 19, wherein the layer(s) or coating(s) and the surface thereunder are being stretched/elongated by 5%, optionally 10%, optionally 20%, optionally 30%, optionally 40%, optionally 50%, optionally 70%, optionally 90%, optionally 100%, optionally 150%, optionally 200% of the original size.
21. The pharmaceutical package or vessel of paragraph 2, wherein the layer(s) or coating(s) is effective to block extractables/leachables from the substrate and any coatings thereon and maintain the blocking properties after being stretched/elongated.
22. The pharmaceutical package or vessel of paragraph 21, wherein the coating(s) and the surface under there is being stretched/elongated by 5%, optionally 10%, optionally 20%, optionally 30%, optionally 40%, optionally 50%, optionally 70%, optionally 90%, optionally 100%, optionally 150%, optionally 200% of the original size.
23. The pharmaceutical package or vessel of paragraph 1, wherein the polymeric wall comprises a film material selected from the group consisting of a polyolefin, a cyclic olefin polymer, a cyclic olefin copolymer, a polypropylene, a polyester, a polyethylene terephthalate (commonly abbreviated PET, PETE, or the obsolete PETP or PET-P PET), a polyethylene naphthalate, a polycarbonate, a polylactic acid, an ethylene vinyl acetate (EVA), an ultra low density polyethylene (ULDPE), a linear low density polyethylene (LLDPE), a polyethylene vinyl alcohol-copolymers (EVOH), an Ethylene-vinyl acetate (EVA) material, a polyamide (PA) polymer, a synthetic polymer (such as polyamide or Nylon), an aliphatic polyamide, a semi-aromatic polyamide, a styrenic polymer or co-polymer, or any combination, composite or blend of any two or more thereof.
24. The pharmaceutical package or vessel of paragraph 1, wherein the package or vessel is a rigid container.
25. A pharmaceutical package or vessel used for CAR-T cell therapy including CAR-T cell manufacturing or treatment comprising:
   a polymeric wall having an interior surface and an outer surface;
   a tie coating or layer of SiOxCy, wherein x is from about 0.5 to about 2.4 and y is from about 0.6 to about 3, on the interior surface of the wall;
   a barrier coating or layer of SiOx, wherein x is from 1.5 to 2.9, on the tie coating or layer of SiOxCy; and
   a passivation coating or layer or pH protective coating or layer of SiOxCy or SiNxCy, wherein x is from about 0.5 to about 2.4 and y is from about 0.6 to about 3, on the innermost surface of the barrier coating or layer.
26. The pharmaceutical package or vessel of paragraph 25, wherein the coating(s) and the surface thereunder are being stretched/elongated by 5%, optionally 10%, optionally 25%, optionally 30%, optionally 40%, optionally 50%, optionally 70%, optionally 90%, optionally 100%, optionally 150%, optionally 200% of the original size.
27. The pharmaceutical package or vessel of paragraph 25, wherein the package or vessel is a bioprocess bag or a transfer bag, or a bag; or a tube, a stopper, or a connector.
28. The pharmaceutical package or vessel of paragraph 25, wherein the package or vessel is a rigid container.
29. A pharmaceutical package or vessel, used for CAR-T cell therapy including CAR-T cell manufacturing and treatment comprising:
   a polymeric wall having an interior surface and an outer surface; and
   a passivation layer or coating or pH protective layer or coating of SiOxCy or SiNxCy, wherein x is from about 0.5 to about 2.4 and y is from about 0.6 to about 3, on the interior surface of the wall.
30. The pharmaceutical package or vessel of paragraph 29, wherein the package or vessel is flexible or stretchable.
31. The pharmaceutical package or vessel of paragraph 29, wherein the package or vessel is a bag, a bioprocess bag or a transfer bag.
32. The pharmaceutical package or vessel of paragraph 30, wherein the coating(s) is able to maintain its desirable characteristics against stretching/elongation conditions.
33. The pharmaceutical package or vessel of paragraph 29, wherein the package or vessel contains a rigid structure.
34. The pharmaceutical package or vessel of paragraph 32, after the coating and the surface thereunder being stretched/elongated by 5%, optionally 10%, optionally 20%, optionally 25%, optionally 30%, optionally 40%, optionally 50%, optionally 70%, optionally 90%, optionally 100%, optionally 150%, optionally 200% of the original size.
35. The pharmaceutical package or vessel of paragraph 29, wherein the pharmaceutical package or vessel is a rigid container.
36. A method of handling a silicon-based coating coated pharmaceutical package or vessel, comprising limiting stretching during manufacturing, packaging, filling, processing and transporting of the packages or vessels.
37. The method of paragraph 36, wherein the silicon-based coating comprises:
   a tie coating or layer of SiOxCy, wherein x is from about 0.5 to about 2.4 and y is from about 0.6 to about 3, on the interior surface of the wall; and/or
   a barrier coating or layer of SiOx, wherein x is from 1.5 to 2.9, on the interior surface of the wall, or when present, on the tie coating or layer of SiOxCy; and/or
   a passivation coating or layer or pH protective coating or layer of SiOxCy or SiNxCy, wherein x is from about 0.5 to about 2.4 and y is from about 0.6 to about 3, on the interior surface of the wall or, when present, on the innermost surface of the tie coating or layer or the barrier coating or layer; and/or
   a surface layer or coating of any of, or combination of, the following:
      silicon-based barrier coating system;
      amorphous carbon coating;
      fluorocarbon coating;
      direct fluorination;
      antiscratch/antistatic coating;
      antistatic coating;
      antistatic additive compound in polymer;
      oxygen scavenging additive compound in polymer;
      colorant additive compound in polymer;
      or antioxidation additive compound in polymer.
38. The method of paragraph 36, wherein the limiting stretching comprises avoiding folding or avoiding sharp creases, optionally placing the package or vessel in
   a tube or sleeve; or
   a rigid frame, optionally made of stainless steel; or
   a flexible intermediate bulk container (FIBC), optionally made of a woven fabric, optionally with four loops on each of the top four corners; or
   on a pallet, optionally lifted up from underneath.
39. The method of paragraph 36, wherein the packages or vessels when filled with contents weigh from: 0 to about 5000 pounds, 0 to about 3000 pounds, 0 to about 2000 pounds, 0 to about 1000 pounds, 0 to about 500 pounds, 0 to about 100 pounds, 0 to about 50 pounds, 0 to about 25 pounds, 0 to about 10 pounds, 0 to about 5 pounds, or 0 to about 1 pound.
40. The method of paragraph 36, wherein the packages or vessels, optionally with the handling tools are moved by robot or overhead gantry system.
41. A pharmaceutical package or vessel of paragraph 1, further comprising a pressure device.
42. The pharmaceutical package or vessel of paragraph 41, wherein the package is a single use bioreactor bag.
43. The pharmaceutical package or vessel of paragraph 41, wherein the pressure device is a pressure monitor.
44. The pharmaceutical package or vessel of paragraph 43, wherein the pressure monitor is capable of monitoring the pressure from 0 to about 1 psi.
45. The pharmaceutical package or vessel of paragraph 43, wherein the pressure monitor is compatible with gamma sterilization.
46. The pharmaceutical package or vessel of paragraph 41, wherein the pressure device is a pressure relief valve or a check valve.
47. The pharmaceutical package or vessel of paragraph 41, which has at least one port.
48. The pharmaceutical package or vessel of paragraph 47, wherein the pressure device is installed in one of the ports.
49. The pharmaceutical package or vessel of any one of the preceding paragraphs, wherein the coating(s) is able to maintain its desirable characteristics during multiple freezing/thawing processes.
50. The pharmaceutical package or vessel of any one of the preceding paragraphs, wherein any pharmaceutical material contained in the package or vessel is able to maintain its integrity during multiple freezing/thawing processes.

An aspect of the disclosure is a bioprocessing or transfer vessel comprising a wall and a barrier coating or layer applied on the wall. Optionally a passivation layer or pH protective coating may be contained on the wall, either directly on the wall or on the barrier coating or layer. The vessel may further contain a fluid composition, such as a gas, liquid, powder, or other composition.

The wall may be initially produced as a film, such as a polymeric film, and then configured and processed into a vessel, such as a bioprocessing or transfer bag or a bag used for CAR-T cell therapy including CAR-T cell manufacturing or treatment. The barrier coating or layer, and/or the passivation layer or pH protective coating, may be applied when the wall is in its film form or after configured into the vessel form. A number of processes may be used to format or manufacture the film into one or more walls of a vessel. A method or process of the present disclosure utilizes welding, particularly laser welding. Laser welding of plastic parts has established itself as a robust, flexible and precise joining process. Laser welding enables highly efficient and flexible assembly from a small-scale production of parts with complex geometries to a high volume industrial manufacturing, where it can be easily integrated into automation lines. This highly repeatable and clean process with no relative parts movement during the welding cycle offers numerous advantages. Thanks to its localized heat input and low mechanical stresses, this process enables welding of sensitive assemblies in medical device manufacturing, industrial and consumer electronics and automotive components without damaging delicate inner components by heat or vibrations.

“Facts About Chimeric Antigen Receptor (CAR)T-Cell Therapy” published by Leukemia & Lymphoma Society, revised June 2018 has described the concept of CAR T cell therapy and the process of CAR T cell manufacturing.

The barrier coating or layer comprises SiOx, wherein x is from 1.5 to 2.9, from 2 to 1000 nm thick. The barrier coating or layer of SiOx can have an interior surface facing the lumen and an outer surface facing the wall interior surface.

The passivation layer or pH protective coating comprises SiOxCy or SiNxCy wherein x is from about 0.5 to about 2.4 and y is from about 0.6 to about 3. Optionally in one embodiment, x can be about 1.1 and y can be about 1.1. The passivation layer or pH protective coating can have an interior surface facing the lumen and an outer surface facing the interior surface of the barrier coating or layer. The passivation layer or pH protective coating can be effective to increase the calculated shelf life of the package (total Si/Si dissolution rate).

The passivation layer or pH protective coating comprises SiOxCy or SiNxCy wherein x is from about 0.5 to about 2.4 and y is from about 0.6 to about 3. The passivation layer or pH protective coating can have an interior surface facing the lumen and an outer surface facing the interior surface of the barrier coating or layer. The passivation layer or pH protective coating can be effective to decrease the Si dissolution rate of the barrier coating or layer.

In at least one embodiment, a pharmaceutical package or vessel, for example a bioprocess bag or a transfer bag or a bag used for CAR-T cell therapy including CAR-T cell manufacturing or treatment, comprises:
a polymeric wall having an interior surface and an outer surface;
a tie coating or layer of SiOxCy, wherein x is from about 0.5 to about 2.4 and y is from about 0.6 to about 3, on the interior surface of the wall; and/or
a barrier coating or layer of SiOₓ, wherein x is from 1.5 to 2.9, on the interior surface of the wall, or when present, on the tie coating or layer of SiOxCy; and/or
a passivation layer or pH protective coating of SiOxCy or SiNxCy, wherein x is from about 0.5 to about 2.4 and y is from about 0.6 to about 3, on the interior surface of the wall or, when present, in the innermost surface of the tie coating or layer or the barrier coating or layer of SiOₓ; and/or
a surface layer or coating of any of, or combination of, the following:
   silicon-based barrier coating system;
   amorphous carbon coating;
   fluorocarbon coating;
   direct fluorination;
   antiscratch/antistatic coating;
   antistatic coating;
   antistatic additive compound in polymer;
   oxygen scavenging additive compound in polymer;
   colorant additive compound in polymer;
   or antioxidation additive compound in polymer,
wherein the coating(s) affords improved barrier properties to gases, moisture and solvents and/or the coating(s) is effective to block extractables/leachables from the substrate and any coatings thereon and/or the coating(s) is able to maintain its desirable characteristics described herein against stretching/elongation conditions.

In at least one embodiment, on the interior surface of the pharmaceutical package or vessel, the coating(s) affords improved barrier properties to gases, moisture and solvents and/or the coating(s) is effective to block extractables/leachables from the substrate and any coatings thereon and/or the coating(s) is able to maintain its blocking properties after the coating(s) and the surface thereunder are being stretched/elongated by 5%, optionally 10%, optionally 20%, optionally 30%, optionally 40%, optionally 50%, optionally 70%, optionally 90%, optionally 100%, optionally 150%, optionally 200% of the original size.

In at least one embodiment, on the interior surface of the pharmaceutical package or vessel, the coating(s) affords improved barrier properties to gases, moisture and solvents and maintains the blocking properties after being stretched/elongated.

In at least one embodiment, on the interior surface of the pharmaceutical package or vessel, the coating(s) affords improved barrier properties to gases, moisture and solvents and maintains the blocking properties after being stretched/elongated by 5%, optionally 10%, optionally 20%, optionally 30%, optionally 40%, optionally 50%, optionally 70%, optionally 90%, optionally 100%, optionally 150%, optionally 200% of the original size.

In at least one embodiment, on the interior surface of the pharmaceutical package or vessel, the coating(s) is effective to block extractables/leachables from the substrate and any coatings thereon and maintains the blocking properties after being stretched/elongated.

In at least one embodiment, on the interior surface of the pharmaceutical package or vessel, the coating(s) is effective to block extractables/leachables from the substrate and any coatings thereon and maintains the blocking properties after the coating(s) and the surface under there being stretched/elongated by 5%, optionally 10%, optionally 20%, optionally 30%, optionally 40%, optionally 50%, optionally 70%, optionally 90%, optionally 100%, optionally 150%, optionally 200% of the original size.

In at least one embodiment, the pharmaceutical package or vessel is, for example, a bioprocess bag or a transfer bag or a bag used for CAR-T cell therapy including CAR-T cell manufacturing or treatment, comprising:
a polymeric wall having an interior surface and an outer surface;
a tie coating or layer of SiOxCy, wherein x is from about 0.5 to about 2.4 and y is from about 0.6 to about 3, on the interior surface of the wall;
a barrier coating or layer of SiOₓ, wherein x is from 1.5 to 2.9, on the tie coating or layer of SiOxCy; and
a passivation layer or pH protective coating of SiOₓCy or SiNₓCy, wherein x is from about 0.5 to about 2.4 and y is from about 0.6 to about 3, on the barrier coating or layer of SiOₓ;
wherein the coatings are effective to block extractables/leachables from the substrate and any coatings thereon when the coatings and the surface thereunder are not being stretched or after being stretched/elongated.

In at least one embodiment, the pharmaceutical package or vessel is, for example, a bioprocess bag or a transfer bag or a bag used for CAR-T cell therapy including CAR-T cell manufacturing or treatment, comprising:
a polymeric wall having an interior surface and an outer surface;
a tie coating or layer of SiOxCy, wherein x is from about 0.5 to about 2.4 and y is from about 0.6 to about 3, on the interior surface of the wall;
a barrier coating or layer of SiOₓ, wherein x is from 1.5 to 2.9, on the tie coating or layer of SiOxCy; and
a passivation layer or pH protective coating of SiOₓCy or SiNₓCy, wherein x is from about 0.5 to about 2.4 and y is from about 0.6 to about 3, on the barrier coating or layer of SiOₓ;
wherein the coatings are effective to block extractables/leachables from the substrate and any coatings thereon after the coatings and the surface thereunder being stretched/elongated by 5%, optionally 10%, optionally 25%, optionally 30%, optionally 40%, optionally 50%, optionally 70%, optionally 90%, optionally 100%, optionally 150%, optionally 200% of the original size.

In at least one embodiment, the pharmaceutical package or vessel is, for example, a bioprocess bag or a transfer bag or a bag used for CAR-T cell therapy including CAR-T cell manufacturing or treatment, comprising:
a polymeric wall having an interior surface and an outer surface; and
a passivation layer or pH protective coating of SiOₓCy or SiNₓCy, wherein x is from about 0.5 to about 2.4 and y is from about 0.6 to about 3, on the interior surface of the wall;
wherein the coating is effective to block extractables/leachables from the substrate after the coating and the surface thereunder being stretched/elongated by 5%, optionally 10%, optionally 20%, optionally 25%, optionally 30%, optionally 40%, optionally 50%, optionally 70%, optionally 90%, optionally 100%, optionally 150%, optionally 200% of the original size.

In at least one embodiment, the package or vessel is a tube, a stopper, or a connector.

In at least one embodiment, the film, wall, or vessel is coated with a barrier coating system which improves the barrier to oxygen, DMSO and moisture and thereby extends the shelf life time of the contained sample. The barrier coating system may include a tie coating or layer of SiOxCy, wherein x is from about 0.5 to about 2.4 and y is from about 0.6 to about 3, each as determined by X-ray photoelectron spectroscopy (XPS); a barrier coating or layer of SiOx, wherein x is from 1.5 to 2.9 as determined by XPS, between the tie coating or layer and the lumen; and optionally, a pH protective coating or layer of SiOxCy, wherein x is from about 0.5 to about 2.4 and y is from about 0.6 to about 3, each as determined by XPS, between the barrier coating or layer and the lumen.

The fluid composition can be contained in the lumen and can have a pH between 4 and 10, alternatively between 5 and 9.

Still another aspect of the disclosure can be an article comprising a wall, a barrier coating or layer, and a passivation layer or pH protective coating.

The barrier coating or layer comprises SiOx, wherein x is from 1.5 to 2.9, from 2 to 1000 nm thick. The barrier coating or layer of SiOx can have an interior surface facing the lumen and an outer surface facing the wall interior surface. The barrier coating or layer can be effective to reduce the ingress of atmospheric gas through the wall compared to an uncoated wall.

The passivation layer or pH protective coating can be on the barrier coating or layer, optionally with one or more intervening layers, and comprises SiOxCy or SiNxCy wherein x is from about 0.5 to about 2.4 and y is from about 0.6 to about 3. The passivation layer or pH protective coating can be formed by chemical vapor deposition of a precursor selected from a linear siloxane, a monocyclic siloxane, a polycyclic siloxane, a polysilsesquioxane, a linear silazane, a monocyclic silazane, a polycyclic silazane, a polysilsesquiazane, a silatrane, a silquasilatrane, a silproatrane, an azasilatrane, an azasilquasiatrane, an azasilproatrane, or a combination of any two or more of these precursors. The rate of erosion of the passivation layer or pH protective coating, if directly contacted by a fluid composition having a pH between 4 and 10, alternatively between 5 and 9, can be less than the rate of erosion of the barrier coating or layer, if directly contacted by the fluid composition.

Other precursors and methods can be used to apply the pH protective coating or layer or passivating treatment. Similarly, these can be used as a separate surface coatings or layers in addition to or as an alternative to the pH protective coatings or layers described above. To accommodate the latter format, these layers and coatings are referred to herein as surface layers and coatings but may be described herein as a passivation or pH protective treatment. For example, hexamethylene disilazane (HMDZ) can be used as the precursor. Another way of applying the pH protective coating or layer is to apply as the pH protective coating or layer an amorphous carbon or fluorocarbon coating (or a fluorinated hydrocarbon coating), or a combination of the two. Amorphous carbon coatings can be formed by PECVD using a saturated hydrocarbon, (e.g. methane or propane) or an unsaturated hydrocarbon (e.g. ethylene, acetylene) as a precursor for plasma polymerization. Fluorocarbon coatings (or a fluorinated hydrocarbon coating) can be derived from fluorocarbons (for example, hexafluoroethylene or tetrafluoroethylene). Either type of coating, or a combination of both, can be deposited by vacuum PECVD or atmospheric pressure PECVD.

It is further contemplated that fluorosilicon precursors can be used to provide a pH protective coating or layer over an SiOx barrier layer. This can be carried out by using as a precursor a fluorinated silane precursor such as hexafluorosilane and a PECVD process. The resulting coating would also be expected to be a non-wetting coating. It is further contemplated that any embodiment of the pH protective coating or layer processes described in this specification can also be carried out without using the article to be coated to contain the plasma.

Yet another coating modality contemplated for protecting or passivating an SiOx barrier layer is coating the barrier layer using a polyamidoamine epichlorohydrin resin. For example, the barrier coated part can be dip coated in a fluid polyamidoamine epichlorohydrin resin melt, solution or dispersion and cured by autoclaving or other heating at a temperature between 60 and 100° C. It is contemplated that a coating of polyamidoamine epichlorohydrin resin can be preferentially used in aqueous environments between pH 5-8, as such resins are known to provide high wet strength in paper in that pH range. Wet strength is the ability to maintain mechanical strength of paper subjected to complete water soaking for extended periods of time, so it is contemplated that a coating of polyamidoamine epichlorohydrin resin on an SiOx barrier layer will have similar resistance to dissolution in aqueous media. It is also contemplated that, because polyamidoamine epichlorohydrin resin imparts a lubricity improvement to paper, it will also provide lubricity in the form of a coating on a thermoplastic surface made of, for example, COC or COP.

Even another approach for protecting a SiOx layer is to apply as a pH protective coating or layer a liquid-applied coating of a polyfluoroalkyl ether, followed by atmospheric plasma curing the pH protective coating or layer. For example, it is contemplated that the process practiced under the trademark TriboGlide®, described in this specification, can be used to provide a pH protective coating or layer that is also a lubricity layer, as TriboGlide® is conventionally used to provide lubricity.

The surface layers and coatings, and the pH protection or passivation coatings and layers, are described herein as protecting an SiOx layer or coating; but that is not required for the embodiments of the present disclosure. The surface layers and coatings, and the pH protection or passivation coatings and layers, may be applied directly to a surface of the wall of the vessel or container or other surface, such as a film or bag.

The preferred drug contact surface includes a coating or layer that provides flexibility while retaining the desirable characteristics of the coatings or layers described herein, including but not limited to moisture barrier, resistance to degradation, compatibility, and the like. Of particular interest is a coating or layer that can provide 1×, 10×, 100×, or larger stretch and elongation of the underlying surface, wall, or film, without detrimentally reducing the desirable characteristics of the coatings or layers described herein, including but not limited to moisture barrier, resistance to degradation, compatibility, and the like. Accordingly, while the embodiments of the present disclosure provide one or more such coatings and layers, other coatings and layers may be contemplated within the scope and breadth of the current disclosure.

The laser welding method of the present disclosure uses a laser beam to melt the plastic in the joint area by delivering a controlled amount of energy to a precise location. This level of precision in controlling the heat input is based on the ease of adjusting the beam size and the range of methods available for precise positioning and moving the beam. The process is based on the same basic requirements of material compatibility as other plastic welding techniques, but is often found to be more forgiving of resin chemistry and melt temperature differences than most other plastic welding processes. Nearly all thermoplastics can be welded using a proper laser source and appropriate joint design.

The adjoining parts, or parts of the vessel that are intended to be joined may be pre-assembled and clamped together to provide intimate contact between their joining surfaces. The laser beam is delivered to the parts interface through the upper “transparent” part and is absorbed by the lower absorbing part, which converts infra-red (IR) energy into heat. The heat is conducted from the lower absorbing part to the upper part allowing the melt to propagate through the interface and form a bond. Precise positioning and clamping of the assembly is essential, as intimate contact is required for heat transfer between the parts. Carbon black and specially designed absorbers may be blended into resin or applied to the surface to enable IR radiation absorption in the lower part of assembly. Some techniques are dependent on the presence of an absorbing agent in the lower component, and this limits the process applicability for manufacturing of medical devices, electronics and some consumer goods when a “clear-to-clear” or a “clear-to-colored” assembly is required.

New laser welding processes reduce, mitigate, or avoid the use of an absorbing agent, such as by utilizing smaller dimension lasers. For example, one or more 2 micron lasers can be utilized to produce the laser weld desired, particularly when a “clear-to-clear” or a “clear-to-colored” assembly is required. This laser is characterized by a greatly increased absorption by clear polymers and enables a highly controlled melting through the thickness of optically clear parts. This has resulted in a greatly improved and simplified technique for laser welding of clear polymers for the medical device industry, which now can fully capitalize on benefits of this advanced assembly process.

The new laser welding processes provide a number of benefits. The laser welding process provides minimal to no flash (e.g., excess polymer material around the weld location), ensuring an aesthetically desired appearance. The process also reduces or removes particulate matter, residue, or other debris generation. Because of the unique laser welding approach, only localized heat input is needed or generated ensuring the structural integrity and performance of the package. Similarly, the non-contact process creates minimal mechanical stress levels on inner components during the weld and reduced residual stress, while still producing excellent bond strength and long-term stability. With this process, complex shapes can be welded to produce the desired package configuration while still ensuring that hermetic seals are achieved.

A broad range of tools may be utilized for the laser welding process. Ideal tools will have a number of features which enable the desired processing of the pharmaceutical vessels. The tools or machine equipment should ideally be non-contact, providing minimized tool wear and retooling cost. They should provide process adjustability and precision, with high process repeatability. Repeatability preferably includes highly controlled and consistent heat input, and precision clamping with no relative motion of parts during the welding cycle, to assure a highly repeatable welding process and consistent joint quality. This results in reduced scrap and quality control costs. Such tools and processing equipment may be readily available, including those commercially available from Dukane IAS, LLC of St. Charles, Ill. The technology from Dukane IAS, LLC utilizes fiber-optic cable, scan head with mirrors coated for appropriate wave length, focusing optics, and programmable multi-axis servo stages for accurate and reproducible laser beam delivery. Dukane systems utilize servo motors to move and precisely position the laser when larger parts are welded. Servo technology can also be used to move the part instead of the laser beam to simplify beam delivery options and reduce system cost while preserving the ability to weld large parts. These capabilities provide an ideal option for tooling capable of producing the laser welding described herein for the production of pharmaceutical packages, particularly bioprocessing bags or transfer bags or a bag used for CAR-T cell therapy including CAR-T cell manufacturing or treatment.

Optionally, the pharmaceutical package comprises a vessel, such as a bioprocessing bag or a transfer bag or a bag used for CAR-T cell therapy including CAR-T cell manufacturing or treatment, having a wall comprising one or more films. In at least one embodiment, the wall comprises a multi-layer film. The film is put on a roll. The coatings or treatments described herein are then applied using a reel-to-reel PECVD coating process (aka roll-to-roll process) where the coating is applied to at least one side of the film, such as the interior surface of the film or wall. The fabrication of the film(s) can be achieved using full roll-to-roll (R2R) processes by, for example, either: (i) in a discrete process configuration of one or more machines where each step (e.g., each coating or layer if one or more coatings or layers are applied) can be applied on separate roll-to-roll setups in series or in sequence, or (ii) in an inline process configuration where all the steps (e.g., each coating or layer is applied in one machine all at the same time or in sequence. The main difference is the number of machines (pairs of starting rolls and finished rolls) used to achieve the final finished roll product.

Once the film is formed, and optionally coated with one or more coatings or layers, the film may be formed into an intermediate or final configuration—such as a bag. One or more of the methods described herein may be used to form the desired configuration, such as by heat staking, fusing, sewing, hot molding, cold molding, injection molding, extrusion, welding, ultrasonic welding, or laser welding (including, as described herein). The desired configuration may be formed before or after the coating stages or steps are performed. If the forming is to occur after the coating stages or steps, i.e., once a coating or layer of SiOx, SiOxCy, and/or SiNxCy is applied, the final shape may be achieved by a number of methods. In at least one embodiment, the coated film may be cuffed (i.e., bent over itself) such that plastic substrate surfaces (instead of the coated surfaces) are able to contact each other and then joined such as by heat staking, fusing, sewing, hot molding, cold molding, injection molding, extrusion, welding, ultrasonic welding, or laser welding. Alternatively, a method such as high speed laser welding (e.g., femtosecond laser welding) could be used to join either the plastic substrate surfaces or the coated surfaces.

Additionally or alternatively, the film could be masked, either passively or actively, during the coating process to enable suitable surfaces to be joined to form the desired configuration. For example, active masking such as with a tape, removable or irremovable coating or layer, or other material that prevents a coating or layer of SiOx, SiOxCy, and/or SiNxCy from being applied to the substrate may be used to enable suitable surfaces to be joined to form the desired configuration. Additionally or alternatively, passive masking such as computer-assisted coaters or detectors may be utilized to ensure certain areas of the film are not coated. For example, the coatings systems may use computers to preserve certain portions, such as edge portions for example, of the film from receiving one or more coatings. The computers may be preprogrammed to identify the uncoated locations of the film. Additionally or alternatively, detectors such as mechanical or optical detectors may be utilized to preserve or identify uncoated portions of the substrate surface. Once the films are processed and the uncoated portions are identified, the plastic substrate surfaces (instead of the coated surfaces) are able to contact each other and then joined such as by heat staking, fusing, sewing, hot molding, cold molding, injection molding, extrusion, welding, ultrasonic welding, or laser welding. The entire film manufacturing, coating, masking, joining, and final forming of the desired configuration may be achieved in one or more machines, such as the roll-to-roll processes described herein.

The vessels, packages, bags, or other surfaces as previously described may contain a fluid. The fluid may comprise, but is not limited to, a member selected from the group consisting of:

### Inhalation Anesthetics

Aliflurane; Chloroform; Cyclopropane; Desflurane (Suprane); Diethyl Ether; Enflurane (Ethrane); Ethyl Chloride; Ethylene; Halothane (Fluothane); Isoflurane (Forane, Isoflo); Isopropenyl vinyl ether; Methoxyflurane; methoxyflurane; Methoxypropane; Nitrous Oxide; Roflurane; Sevoflurane (Sevorane, Ultane, Sevoflo); Teflurane; Trichloroethylene; Vinyl Ether; Xenon.

### Injectable Drugs

Ablavar (Gadofosveset Trisodium Injection); Abarelix Depot; Abobotulinumtoxin A Injection (Dysport); ABT-263; ABT-869; ABX-EFG; Accretropin (Somatropin Injection); Acetadote (Acetylcysteine Injection); Acetazolamide Injection (Acetazolamide Injection); Acetylcysteine Injection (Acetadote); Actemra (Tocilizumab Injection); Acthrel (Corticorelin Ovine Triflutate for Injection); Actummune; Activase; Acyclovir for Injection (Zovirax Injection); Adacel; Adalimumab; Adenoscan (Adenosine Injection); Adenosine Injection (Adenoscan); Adrenaclick; AdreView (Iobenguane 1123 Injection for Intravenous Use); Afluria; Ak-Fluor (Fluorescein Injection); Aldurazyme (Laronidase); Alglucerase Injection (Ceredase); Alkeran Injection (Melphalan Hcl Injection); Allopurinol Sodium for Injection (Aloprim); Aloprim (Allopurinol Sodium for Injection); Alprostadil; Alsuma (Sumatriptan Injection); ALTU-238; Amino Acid Injections; Aminosyn; Apidra; Apremilast; Alprostadil Dual Chamber System for Injection (Caverject Impulse); AMG 009; AMG 076; AMG 102; AMG 108; AMG 114; AMG 162; AMG 220; AMG 221; AMG 222; AMG 223; AMG 317; AMG 379; AMG 386; AMG 403; AMG 477; AMG 479; AMG 517; AMG 531; AMG 557; AMG 623; AMG 655; AMG 706; AMG 714; AMG 745; AMG 785; AMG 811; AMG 827; AMG 837; AMG 853; AMG 951; Amiodarone HCl Injection (Amiodarone HCl Injection); Amobarbital Sodium Injection (Amytal Sodium); Amytal Sodium (Amobarbital Sodium Injection); Anakinra; Anti-Abeta; Anti-Beta7; Anti-Beta20; Anti-CD4; Anti-CD20; Anti-CD40; Anti-IFNalpha; Anti-IL13; Anti-OX40L; Anti-oxLDS; Anti-NGF; Anti-NRP1; Arixtra; Amphadase (Hyaluronidase Inj); Ammonul (Sodium Phenylacetate and Sodium Benzoate Injection); Anaprox; Anzemet Injection (Dolasetron Mesylate Injection); Apidra (Insulin Glulisine [rDNA origin] Inj); Apomab; Aranesp (darbepoetin alfa); Argatroban (Argatroban Injection); Arginine Hydrochloride Injection (R-Gene 10); Aristocort; Aristospan; Arsenic Trioxide Injection (Trisenox); Articane HCl and Epinephrine Injection (Septocaine); Arzerra (Ofatumumab Injection); Asclera (Polidocanol Injection); Ataluren; Ataluren-DMD; Atenolol Inj (Tenormin I.V. Injection); Atracurium Besylate Injection (Atracurium Besylate Injection); Avastin; Azactam Injection (Aztreonam Injection); Azithromycin (Zithromax Injection); Aztreonam Injection (Azactam Injection); Baclofen Injection (Lioresal Intrathecal); Bacteriostatic Water (Bacteriostatic Water for Injection); Baclofen Injection (Lioresal Intrathecal); Bal in Oil Ampules (Dimercarprol Injection); BayHepB; BayTet; Benadryl; Bendamustine Hydrochloride Injection (Treanda); Benztropine Mesylate Injection (Cogentin); Betamethasone Injectable Suspension (Celestone Soluspan); Bexxar; Bicillin C-R 900/300 (Penicillin G Benzathine and Penicillin G Procaine Injection); Blenoxane (Bleomycin Sulfate Injection); Bleomycin Sulfate Injection (Blenoxane); Boniva Injection (Ibandronate Sodium Injection); Botox Cosmetic (OnabotulinumtoxinA for Injection); BR3-FC; Bravelle (Urofollitropin Injection); Bretylium (Bretylium Tosylate Injection); Brevital Sodium (Methohexital Sodium for Injection); Brethine; Briobacept; BTT-1023; Bupivacaine HCl; Byetta; Ca-DTPA (Pentetate Calcium Trisodium Inj); Cabazitaxel Injection (Jevtana); Caffeine Alkaloid (Caffeine and Sodium Benzoate Injection); Calcijex Injection (Calcitrol); Calcitrol (Calcijex Injection); Calcium Chloride (Calcium Chloride Injection 10%); Calcium Disodium Versenate (Edetate Calcium Disodium Injection); Campath (Altemtuzumab); Camptosar Injection (Irinotecan Hydrochloride); Canakinumab Injection (Ilaris); Capastat Sulfate (Capreomycin for Injection); Capreomycin for Injection (Capastat Sulfate); Cardiolite (Prep kit for Technetium Tc99 Sestamibi for Injection); Carticel; Cathflo; Cefazolin and Dextrose for Injection (Cefazolin Injection); Cefepime Hydrochloride; Cefotaxime; Cefiriaxone; Cerezyme; Carnitor Injection; Caverject; Celestone Soluspan; Celsior; Cerebyx (Fosphenytoin Sodium Injection); Ceredase (Alglucerase Injection); Ceretec (Technetium Tc99m Exametazime Injection); Certolizumab; CF-101; Chloramphenicol Sodium Succinate (Chloramphenicol Sodium Succinate Injection); Chloramphenicol Sodium Succinate Injection (Chloramphenicol Sodium Succinate); Cholestagel (Colesevelam HCL); Choriogonadotropin Alfa Injection (Ovidrel); Cimzia; Cisplatin (Cisplatin Injection); Clolar (Clofarabine Injection); Clomiphine Citrate; Clonidine Injection (Duraclon); Cogentin (Benztropine Mesylate Injection); Colistimethate Injection (Coly-Mycin M); Coly-Mycin M (Colistimethate Injection); Compath; Conivaptan Hcl Injection (Vaprisol); Conjugated Estrogens for Injection (Premarin Injection); Copaxone; Corticorelin Ovine Triflutate for Injection (Acthrel); Corvert (Ibutilide Fumarate Injection); Cubicin (Daptomycin Injection); CF-101; Cyanokit (Hydroxocobalamin for Injection); Cytarabine Liposome Injection (DepoCyt); Cyanocobalamin; Cytovene (ganciclovir); D.H.E. 45; Dacetuzumab; Dacogen (Decitabine Injection); Dalteparin; Dantrium IV (Dantrolene Sodium for Injection); Dantrolene Sodium for Injection (Dantrium IV); Daptomycin Injection (Cubicin); DarbepoietinAlfa; DDAVP Injection (Desmopressin Acetate Injection); Decavax; Decitabine Injection (Dacogen); Dehydrated Alcohol (Dehydrated Alcohol Injection); Denosumab Injection (Prolia); Delatestryl; Delestrogen; Delteparin Sodium; Depacon (Valproate Sodium Injection); Depo Medrol (Methylprednisolone Acetate Injectable Suspension); DepoCyt (Cytarabine Liposome Injection); DepoDur (Morphine Sulfate XR Liposome Injection); Desmopressin Acetate Injection (DDAVP Injection); Depo-Estradiol; Depo-Provera 104 mg/ml; Depo-Provera 150 mg/ml; Depo-Testosterone; Dexrazoxane for Injection, Intravenous Infusion Only (Totect); Dextrose/Electrolytes; Dextrose and Sodium Chloride Inj (Dextrose 5% in 0.9% Sodium Chloride); Dextrose; Diazepam Injection (Diazepam Injection); Digoxin Injection (Lanoxin Injection); Dilaudid-HP (Hydromorphone Hydrochloride Injection); Dimercarprol Injection (Bal in Oil Ampules); Diphenhydramine Injection (Benadryl Injection); Dipyridamole Injection (Dipyridamole Injection); DMOAD; Docetaxel for Injection (Taxotere); Dolasetron Mesylate Injection (Anzemet Injection); Doribax (Doripenem for Injection); Doripenem for Injection (Doribax); Doxercalciferol Injection (Hectorol Injection); Doxil (Doxorubicin Hcl Liposome Injection); Doxorubicin Hcl Liposome Injection (Doxil); Duraclon (Clonidine Injection); Duramorph (Morphine Injection); Dysport (Abobotulinumtoxin A Injection); Ecallantide Injection (Kalbitor); EC-Naprosyn (naproxen); Edetate Calcium Disodium Injection (Calcium Disodium Versenate); Edex (Alprostadil for Injection); Engerix; Edrophonium Injection (Enlon); Eliglustat Tartate; Eloxatin (Oxaliplatin Injection); Emend Injection (Fosaprepitant Dimeglumine Injection); Enalaprilat Injection (Enalaprilat Injection); Enlon (Edrophonium Injection); Enoxaparin Sodium Injection (Lovenox); Eovist (Gadoxetate Disodium Injection); Enbrel (etanercept); Enoxaparin; Epicel; Epinepherine; Epipen; Epipen Jr.; Epratuzumab; Erbitux; Ertapenem Injection (Invanz); Erythropoieten; Essential Amino Acid Injection (Nephramine); Estradiol Cypionate; Estradiol Valerate; Etanercept; Exenatide Injection (Byetta); Evlotra; Fabrazyme (Adalsidase beta); Famotidine Injection; FDG (Fludeoxyglucose F 18 Injection); Feraheme (Ferumoxytol Injection); Feridex I.V. (Ferumoxides Injectable Solution); Fertinex; Ferumoxides Injectable Solution (Feridex I.V.); Ferumoxytol Injection (Feraheme); Flagyl Injection (Metronidazole Injection); Fluarix; Fludara (Fludarabine Phosphate); Fludeoxyglucose F 18 Injection (FDG); Fluorescein Injection (Ak-Fluor); Follistim AQ Cartridge (Follitropin Beta Injection); Follitropin Alfa Injection (Gonal-f RFF); Follitropin Beta Injection (Follistim AQ Cartridge); Folotyn (Pralatrexate Solution for Intravenous Injection); Fondaparinux; Forteo (Teriparatide (rDNA origin) Injection); Fostamatinib; Fosaprepitant Dimeglumine Injection (Emend Injection); Foscarnet Sodium Injection (Foscavir); Foscavir (Foscarnet Sodium Injection); Fosphenytoin Sodium Injection (Cerebyx); Fospropofol Disodium Injection (Lusedra); Fragmin; Fuzeon (enfuvirtide); GA101; Gadobenate Dimeglumine Injection (Multihance); Gadofosveset Trisodium Injection (Ablavar); Gadoteridol Injection Solution (ProHance); Gadoversetamide Injection (OptiMARK); Gadoxetate Disodium Injection (Eovist); Ganirelix (Ganirelix Acetate Injection); Gardasil; GC1008; GDFD; Gemtuzumab Ozogamicin for Injection (Mylotarg); Genotropin; Gentamicin Injection; GENZ-112638; Golimumab Injection (Simponi Injection); Gonal-f RFF (Follitropin Alfa Injection); Granisetron Hydrochloride (Kytril Injection); Gentamicin Sulfate; Glatiramer Acetate; Glucagen; Glucagon; HAE1; Haldol (Haloperidol Injection); Havrix; Hectorol Injection (Doxercalciferol Injection); Hedgehog Pathway Inhibitor; Heparin; Herceptin; hG-CSF; Humalog; Human Growth Hormone; Humatrope; HuMax; Humegon; Humira; Humulin; Ibandronate Sodium Injection (Boniva Injection); Ibuprofen Lysine Injection (NeoProfen); Ibutilide Fumarate Injection (Corvert); Idamycin PFS (Idarubicin Hydrochloride Injection); Idarubicin Hydrochloride Injection (Idamycin PFS); Ilaris (Canakinumab Injection); Imipenem and Cilastatin for Injection (Primaxin I.V.); Imitrex; Incobotulinumtoxin A for Injection (Xeomin); Increlex (Mecasermin [rDNA origin] Injection); Indocin IV (Indomethacin Inj); Indomethacin Inj (Indocin IV); Infanrix; Innohep; Insulin; Insulin Aspart [rDNA origin] Inj (NovoLog); Insulin Glargine [rDNA origin] Injection (Lantus); Insulin Glulisine [rDNA origin] Inj (Apidra); Interferon alfa-2b, Recombinant for Injection (Intron A); Intron A (Interferon alfa-2b, Recombinant for Injection); Invanz (Ertapenem Injection); Invega Sustenna (Paliperidone Palmitate Extended-Release Injectable Suspension); Invirase (saquinavir mesylate); Iobenguane 1123 Injection for Intravenous Use (AdreView); Iopromide Injection (Ultravist); Ioversol Injection (Optiray Injection); Iplex (Mecasermin Rinfabate [rDNA origin] Injection); Iprivask; Irinotecan Hydrochloride (Camptosar Injection); Iron Sucrose Injection (Venofer); Istodax (Romidepsin for Injection); Itraconazole Injection (Sporanox Injection); Jevtana (Cabazitaxel Injection); Jonexa; Kalbitor (Ecallantide Injection); KCL in D5NS (Potassium Chloride in 5% Dextrose and Sodium Chloride Injection); KCL in D5W; KCL in NS; Kenalog 10 Injection (Triamcinolone Acetonide Injectable Suspension); Kepivance (Palifermin); Keppra Injection (Levetiracetam); Keratinocyte; KFG; Kinase Inhibitor; Kineret (Anakinra); Kinlytic (Urokinase Injection); Kinrix; Klonopin (clonazepam); Kytril Injection (Granisetron Hydrochloride); lacosamide Tablet and Injection (Vimpat); Lactated Ringer's; Lanoxin Injection (Digoxin Injection); Lansoprazole for Injection (Prevacid I.V.); Lantus; Leucovorin Calcium (Leucovorin Calcium Injection); Lente (L); Leptin; Levemir; Leukine Sargramostim; Leuprolide Acetate; Levothyroxine; Levetiracetam (Keppra Injection); Lovenox; Levocarnitine Injection (Carnitor Injection); Lexiscan (Regadenoson Injection); Lioresal Intrathecal (Baclofen Injection); Liraglutide [rDNA] Injection (Victoza); Lovenox (Enoxaparin Sodium Injection); Lucentis (Ranibizumab Injection); Lumizyme; Lupron (Leuprolide Acetate Injection); Lusedra (Fospropofol Disodium Injection); Maci; Magnesium Sulfate (Magnesium Sulfate Injection); Mannitol Injection (Mannitol IV); Marcaine (Bupivacaine Hydrochloride and Epinephrine Injection); Maxipime (Cefepime Hydrochloride for Injection); MDP Multidose Kit of Technetium Injection (Technetium Tc99m Medronate Injection); Mecasermin [rDNA origin] Injection (Increlex); Mecasermin Rinfabate [rDNA origin] Injection (Iplex); Melphalan Hcl Injection (Alkeran Injection); Methotrexate; Menactra; Menopur (Menotropins Injection); Menotropins for Injection (Repronex); Methohexital Sodium for Injection (Brevital Sodium); Methyldopate Hydrochloride Injection, Solution (Methyldopate Hcl); Methylene Blue (Methylene Blue Injection); Methylprednisolone Acetate Injectable Suspension (Depo Medrol); MetMab; Metoclopramide Injection (Reglan Injection); Metrodin (Urofollitropin for Injection); Metronidazole Injection (Flagyl Injection); Miacalcin; Midazolam (Midazolam Injection); Mimpara (Cinacalet); Minocin Injection (Minocycline Inj); Minocycline Inj (Minocin Injection); Mipomersen; Mitoxantrone for Injection Concentrate (Novantrone); Morphine Injection (Duramorph); Morphine Sulfate XR Liposome Injection (DepoDur); Morrhuate Sodium (Morrhuate Sodium Injection); Motesanib; Mozobil (Plerixafor Injection); Multihance (Gadobenate Dimeglumine Injection); Multiple Electrolytes and Dextrose Injection; Multiple Electrolytes Injection; Mylotarg (Gemtuzumab Ozogamicin for Injection); Myozyme (Alglucosidase alfa); Nafcillin Injection (Nafcillin Sodium); Nafcillin Sodium (Nafcillin Injection); Naltrexone XR Inj (Vivitrol); Naprosyn (naproxen); NeoProfen (Ibuprofen Lysine Injection); Nandrol Decanoate; Neostigmine Methylsulfate (Neostigmine Methylsulfate Injection); NEO-GAA; NeoTect (Technetium Tc 99m Depreotide Injection); Nephramine (Essential Amino Acid Injection); Neulasta (pegfilgrastim); Neupogen (Filgrastim); Novolin; Novolog; NeoRecormon; Neutrexin (Trimetrexate Glucuronate Inj); NPH (N); Nexterone (Amiodarone HCl Injection); Norditropin (Somatropin Injection); Normal Saline (Sodium Chloride Injection); Novantrone (Mitoxantrone for Injection Concentrate); Novolin 70/30 Innolet (70% NPH, Human Insulin Isophane Suspension and 30% Regular, Human Insulin Injection); NovoLog (Insulin Aspart [rDNA origin] Inj); Nplate (romiplostim); Nutropin (Somatropin (rDNA origin) for Inj); Nutropin AQ; Nutropin Depot (Somatropin (rDNA origin) for Inj); Octreotide Acetate Injection (Sandostatin LAR); Ocrelizumab; Ofatumumab Injection (Arzerra); Olanzapine Extended Release Injectable Suspension (Zyprexa Relprevv); Omnitarg; Omnitrope (Somatropin [rDNA origin] Injection); Ondansetron Hydrochloride Injection (Zofran Injection); OptiMARK (Gadoversetamide Injection); Optiray Injection (Ioversol Injection); Orencia; Osmitrol Injection in Aviva (Mannitol Injection in Aviva Plastic Vessel); Osmitrol Injection in Viaflex (Mannitol Injection in Viaflex Plastic Vessel); Osteoprotegrin; Ovidrel (Choriogonadotropin Alfa Injection); Oxacillin (Oxacillin for Injection); Oxaliplatin Injection (Eloxatin); Oxytocin Injection (Pitocin); Paliperidone Palmitate Extended-Release Injectable Suspension (Invega Sustenna); Pamidronate Disodium Injection (Pamidronate Disodium Injection); Panitumumab Injection for Intravenous Use (Vectibix); Papaverine Hydrochloride Injection (Papaverine Injection); Papaverine Injection (Papaverine Hydrochloride Injection); Parathyroid Hormone; Paricalcitol Injection Fliptop Vial (Zemplar Injection); PARP Inhibitor; Pediarix; PEGIntron; Peginterferon; Pegfilgrastim; Penicillin G Benzathine and Penicillin G Procaine; Pentetate Calcium Trisodium Inj (Ca-DTPA); Pentetate Zinc Trisodium Injection (Zn-DTPA); Pepcid Injection (Famotidine Injection); Pergonal; Pertuzumab; Phentolamine Mesylate (Phentolamine Mesylate for Injection); Physostigmine Salicylate (Physostigmine Salicylate (injection)); Physostigmine Salicylate (injection) (Physostigmine Salicylate); Piperacillin and Tazobactam Injection (Zosyn); Pitocin (Oxytocin Injection); Plasma-Lyte 148 (Multiple Electrolytes Inj); Plasma-Lyte 56 and Dextrose (Multiple Electrolytes and Dextrose Injection in Viaflex Plastic Vessel); PlasmaLyte; Plerixafor Injection (Mozobil); Polidocanol Injection (Asclera); Potassium Chloride; Pralatrexate Solution for Intravenous Injection (Folotyn); Pramlintide Acetate Injection (Symlin); Premarin Injection (Conjugated Estrogens for Injection); Prep kit for Technetium Tc99 Sestamibi for Injection (Cardiolite); Prevacid I.V. (Lansoprazole for Injection); Primaxin I.V. (Imipenem and Cilastatin for Injection); Prochymal; Procrit; Progesterone; ProHance (Gadoteridol Injection Solution); Prolia (Denosumab Injection); Promethazine HCl Injection (Promethazine Hydrochloride Injection); Propranolol Hydrochloride Injection (Propranolol Hydrochloride Injection); Quinidine Gluconate Injection (Quinidine Injection); Quinidine Injection (Quinidine Gluconate Injection); R-Gene 10 (Arginine Hydrochloride Injection); Ranibizumab Injection (Lucentis); Ranitidine Hydrochloride Injection (Zantac Injection); Raptiva; Reclast (Zoledronic Acid Injection); Recombivarix HB; Regadenoson Injection (Lexiscan); Reglan Injection (Metoclopramide Injection); Remicade; Renagel; Renvela (Sevelamer Carbonate); Repronex (Menotropins for Injection); Retrovir IV (Zidovudine Injection); rhApo2L/TRAIL; Ringer's and 5% Dextrose Injection (Ringers in Dextrose); Ringer's Injection (Ringers Injection); Rituxan; Rituximab; Rocephin (ceftriaxone); Rocuronium Bromide Injection (Zemuron); Roferon-A (interferon alfa-2a); Romazicon (flumazenil); Romidepsin for Injection (Istodax); Saizen (Somatropin Injection); Sandostatin LAR (Octreotide Acetate Injection); Sclerostin Ab; Sensipar (cinacalcet); Sensorcaine (Bupivacaine HCl Injections); Septocaine (Articane HCl and Epinephrine Injection); Serostim LQ (Somatropin (rDNA origin) Injection); Simponi Injection (Golimumab Injection); Sodium Acetate (Sodium Acetate Injection); Sodium Bicarbonate (Sodium Bicarbonate 5% Injection); Sodium Lactate (Sodium Lactate Injection in AVIVA); Sodium Phenylacetate and Sodium Benzoate Injection (Ammonul); Somatropin (rDNA origin) for Inj (Nutropin); Sporanox Injection (Itraconazole Injection); Stelara Injection (Ustekinumab); Stemgen; Sufenta (Sufentanil Citrate Injection); Sufentanil Citrate Injection (Sufenta); Sumavel; Sumatriptan Injection (Alsuma); Symlin; Symlin Pen; Systemic Hedgehog Antagonist; Synvisc-One (Hylan G-F 20 Single Intra-articular Injection); Tarceva; Taxotere (Docetaxel for Injection); Technetium Tc 99m; Telavancin for Injection (Vibativ); Temsirolimus Injection (Torisel); Tenormin I.V. Injection (Atenolol Inj); Teriparatide (rDNA origin) Injection (Forteo); Testosterone Cypionate; Testosterone Enanthate; Testosterone Propionate; Tev-Tropin (Somatropin, rDNA Origin, for Injection); tgAAC94; Thallous Chloride; Theophylline; Thiotepa (Thiotepa Injection); Thymoglobulin (Anti-Thymocyte Globulin (Rabbit); Thyrogen (Thyrotropin Alfa for Injection); Ticarcillin Disodium and Clavulanate Potassium Galaxy (Timentin Injection); Tigan Injection (Trimethobenzamide Hydrochloride Injectable); Timentin Injection (Ticarcillin Disodium and Clavulanate Potassium Galaxy); TNKase; Tobramycin Injection (Tobramycin Injection); Tocilizumab Injection (Actemra); Torisel (Temsirolimus Injection); Totect (Dexrazoxane for Injection, Intravenous Infusion Only); Trastuzumab-DM1; Travasol (Amino Acids (Injection)); Treanda (Bendamustine Hydrochloride Injection); Trelstar (Triptorelin Pamoate for Injectable Suspension); Triamcinolone Acetonide; Triamcinolone Diacetate; Triamcinolone Hexacetonide Injectable Suspension (Aristospan Injection 20 mg); Triesence (Triamcinolone Acetonide Injectable Suspension); Trimethobenzamide Hydrochloride Injectable (Tigan Injection); Trimetrexate Glucuronate Inj (Neutrexin); Triptorelin Pamoate for Injectable Suspension (Trelstar); Twinject; Trivaris (Triamcinolone Acetonide Injectable Suspension); Trisenox (Arsenic Trioxide Injection); Twinrix; Typhoid Vi; Ultravist (Iopromide Injection); Urofollitropin for Injection (Metrodin); Urokinase Injection (Kinlytic); Ustekinumab (Stelara Injection); Ultralente (U); Valium (diazepam); Valproate Sodium Injection (Depacon); Valtropin (Somatropin Injection); Vancomycin Hydrochloride (Vancomycin Hydrochloride Injection); Vancomycin Hydrochloride Injection (Vancomycin Hydrochloride); Vaprisol (Conivaptan Hcl Injection); VAQTA; Vasovist (Gadofosveset Trisodium Injection for Intravenous Use); Vectibix (Panitumumab Injection for Intravenous Use); Venofer (Iron Sucrose Injection); Verteporfin Inj (Visudyne); Vibativ (Telavancin for Injection); Victoza (Liraglutide [rDNA] Injection); Vimpat (lacosamide Tablet and Injection); Vinblastine Sulfate (Vinblastine Sulfate Injection); Vincasar PFS (Vincristine Sulfate Injection); Victoza; Vincristine Sulfate (Vincristine Sulfate Injection); Visudyne (Verteporfin Inj); Vitamin B-12; Vivitrol (Naltrexone XR Inj); Voluven (Hydroxyethyl Starch in Sodium Chloride Injection); Xeloda; Xenical (orlistat); Xeomin (Incobotulinumtoxin A for Injection); Xolair; Zantac Injection (Ranitidine Hydrochloride Injection); Zemplar Injection (Paricalcitol Injection Fliptop Vial); Zemuron (Rocuronium Bromide Injection); Zenapax (daclizumab); Zevalin; Zidovudine Injection (Retrovir IV); Zithromax Injection (Azithromycin); Zn-DTPA (Pentetate Zinc Trisodium Injection); Zofran Injection (Ondansetron Hydrochloride Injection); Zingo; Zoledronic Acid for Inj (Zometa); Zoledronic Acid Injection (Reclast); Zometa (Zoledronic Acid for Inj); Zosyn (Piperacillin and Tazobactam Injection); Zyprexa Relprevy (Olanzapine Extended Release Injectable Suspension).

### Liquid Drugs (Non-Injectable)

Abilify; AccuNeb (Albuterol Sulfate Inhalation Solution); Actidose Aqua (Activated Charcoal Suspension); Activated Charcoal Suspension (Actidose Aqua); Advair; Agenerase Oral Solution (Amprenavir Oral Solution); Akten (Lidocaine Hydrochloride Ophthalmic Gel); Alamast (Pemirolast Potassium Ophthalmic Solution); Albumin (Human) 5% Solution (Buminate 5%); Albuterol Sulfate Inhalation Solution; Alinia; Alocril; Alphagan; Alrex; Alvesco; Amprenavir Oral Solution; Analpram-HC; Arformoterol Tartrate Inhalation Solution (Brovana); Aristospan Injection 20 mg (Triamcinolone Hexacetonide Injectable Suspension); Asacol; Asmanex; Astepro; Astepro (Azelastine Hydrochloride Nasal Spray); Atrovent Nasal Spray (Ipratropium Bromide Nasal Spray); Atrovent Nasal Spray 0.06; Augmentin ES-600; Azasite (Azithromycin Ophthalmic Solution); Azelaic Acid (Finacea Gel); Azelastine Hydrochloride Nasal Spray (Astepro); Azelex (Azelaic Acid Cream); Azopt (Brinzolamide Ophthalmic Suspension); Bacteriostatic Saline; Balanced Salt; Bepotastine; Bactroban Nasal; Bactroban; Beclovent; Benzac W; Betimol; Betoptic S; Bepreve; Bimatoprost Ophthalmic Solution; Bleph 10 (Sulfacetamide Sodium Ophthalmic Solution 10%); Brinzolamide Ophthalmic Suspension (Azopt); Bromfenac Ophthalmic Solution (Xibrom); Bromhist; Brovana (Arformoterol Tartrate Inhalation Solution); Budesonide Inhalation Suspension (Pulmicort Respules); Cambia (Diclofenac Potassium for Oral Solution); Capex; Carac; Carboxine-PSE; Carnitor; Cayston (Aztreonam for Inhalation Solution); Cellcept; Centany; Cerumenex; Ciloxan Ophthalmic Solution (Ciprofloxacin HCL Ophthalmic Solution); Ciprodex; Ciprofloxacin HCL Ophthalmic Solution (Ciloxan Ophthalmic Solution); Clemastine Fumarate Syrup (Clemastine Fumarate Syrup); CoLyte (PEG Electrolytes Solution); Combiven; Comtan; Condylox; Cordran; Cortisporin Ophthalmic Suspension; Cortisporin Otic Suspension; Cromolyn Sodium Inhalation Solution (Intal Nebulizer Solution); Cromolyn Sodium Ophthalmic Solution (Opticrom); Crystalline Amino Acid Solution with Electrolytes (Aminosyn Electrolytes); Cutivate; Cuvposa (Glycopyrrolate Oral Solution); Cyanocobalamin (CaloMist Nasal Spray); Cyclosporine Oral Solution (Gengraf Oral Solution); Cyclogyl; Cysview (Hexaminolevulinate Hydrochloride Intravesical Solution); DermOtic Oil (Fluocinolone Acetonide Oil Ear Drops); Desmopressin Acetate Nasal Spray; DDAVP; Derma-Smoothe/FS; Dexamethasone Intensol; Dianeal Low Calcium; Dianeal PD; Diclofenac Potassium for Oral Solution (Cambia); Didanosine Pediatric Powder for Oral Solution (Videx); Differin; Dilantin 125 (Phenytoin Oral Suspension); Ditropan; Dorzolamide Hydrochloride Ophthalmic Solution (Trusopt); Dorzolamide Hydrochloride-Timolol Maleate Ophthalmic Solution (Cosopt); Dovonex Scalp (Calcipotriene Solution); Doxycycline Calcium Oral Suspension (Vibramycin Oral); Efudex; Elaprase (Idursulfase Solution); Elestat (Epinastine HCl Ophthalmic Solution); Elocon; Epinastine HCl Ophthalmic Solution (Elestat); Epivir HBV; Epogen (Epoetin alfa); Erythromycin Topical Solution 1.5% (Staticin); Ethiodol (Ethiodized Oil); Ethosuximide Oral Solution (Zarontin Oral Solution); Eurax; Extraneal (Icodextrin Peritoneal Dialysis Solution); Felbatol; Feridex I.V. (Ferumoxides Injectable Solution); Flovent; Floxin Otic (Ofloxacin Otic Solution); Flo-Pred (Prednisolone Acetate Oral Suspension); Fluoroplex; Flunisolide Nasal Solution (Flunisolide Nasal Spray 0.025%); Fluorometholone Ophthalmic Suspension (FML); Flurbiprofen Sodium Ophthalmic Solution (Ocufen); FML; Foradil; Formoterol Fumarate Inhalation Solution (Perforomist); Fosamax; Furadantin (Nitrofurantoin Oral Suspension); Furoxone; Gammagard Liquid (Immune Globulin Intravenous (Human) 10%); Gantrisin (Acetyl Sulfisoxazole Pediatric Suspension); Gatifloxacin Ophthalmic Solution (Zymar); Gengraf Oral Solution (Cyclosporine Oral Solution); Glycopyrrolate Oral Solution (Cuvposa); Halcinonide Topical Solution (Halog Solution); Halog Solution (Halcinonide Topical Solution); HEP-LOCK U/P (Preservative-Free Heparin Lock Flush Solution); Heparin Lock Flush Solution (Hepflush 10); Hexaminolevulinate Hydrochloride Intravesical Solution (Cysview); Hydrocodone Bitartrate and Acetaminophen Oral Solution (Lortab Elixir); Hydroquinone 3% Topical Solution (Melquin-3 Topical Solution); IAP Antagonist; Isopto; Ipratropium Bromide Nasal Spray (Atrovent Nasal Spray); Itraconazole Oral Solution (Sporanox Oral Solution); Ketorolac Tromethamine Ophthalmic Solution (Acular LS); Kaletra; Lanoxin; Lexiva; Leuprolide Acetate for Depot Suspension (Lupron Depot 11.25 mg); Levobetaxolol Hydrochloride Ophthalmic Suspension (Betaxon); Levocarnitine Tablets, Oral Solution, Sugar-Free (Carnitor); Levofloxacin Ophthalmic Solution 0.5% (Quixin); Lidocaine HCl Sterile Solution (Xylocaine MPF Sterile Solution); Lok Pak (Heparin Lock Flush Solution); Lorazepam Intensol; Lortab Elixir (Hydrocodone Bitartrate and Acetaminophen Oral Solution); Lotemax (Loteprednol Etabonate Ophthalmic Suspension); Loteprednol Etabonate Ophthalmic Suspension (Alrex); Low Calcium Peritoneal Dialysis Solutions (Dianeal Low Calcium); Lumigan (Bimatoprost Ophthalmic Solution 0.03% for Glaucoma); Lupron Depot 11.25 mg (Leuprolide Acetate for Depot Suspension); Megestrol Acetate Oral Suspension (Megestrol Acetate Oral Suspension); MEK Inhibitor; Mepron; Mesnex; Mestinon; Mesalamine Rectal Suspension Enema (Rowasa); Melquin-3 Topical Solution (Hydroquinone 3% Topical Solution); MetMab; Methyldopate Hcl (Methyldopate Hydrochloride Injection, Solution); Methylin Oral Solution (Methylphenidate HCl Oral Solution 5 mg/5 mL and 10 mg/5 mL); Methylprednisolone Acetate Injectable Suspension (Depo Medrol); Methylphenidate HCl Oral Solution 5 mg/5 mL and 10 mg/5 mL (Methylin Oral Solution); Methylprednisolone sodium succinate (Solu Medrol); Metipranolol Ophthalmic Solution (Optipranolol); Migranal; Miochol-E (Acetylcholine Chloride Intraocular Solution); Micro-K for Liquid Suspension (Potassium Chloride Extended Release Formulation for Liquid Suspension); Minocin (Minocycline Hydrochloride Oral Suspension); Nasacort; Neomycin and Polymyxin B Sulfates and Hydrocortisone; Nepafenac Ophthalmic Suspension (Nevanac); Nevanac (Nepafenac Ophthalmic Suspension); Nitrofurantoin Oral Suspension (Furadantin); Noxafil (Posaconazole Oral Suspension); Nystatin (oral) (Nystatin Oral Suspension); Nystatin Oral Suspension (Nystatin (oral)); Ocufen (Flurbiprofen Sodium Ophthalmic Solution); Ofloxacin Ophthalmic Solution (Ofloxacin Ophthalmic Solution); Ofloxacin Otic Solution (Floxin Otic); Olopatadine Hydrochloride Ophthalmic Solution (Pataday); Opticrom (Cromolyn Sodium Ophthalmic Solution); Optipranolol (Metipranolol Ophthalmic Solution); Patanol; Pediapred; PerioGard; Phenytoin Oral Suspension (Dilantin 125); Phisohex; Posaconazole Oral Suspension (Noxafil); Potassium Chloride Extended Release Formulation for Liquid Suspension (Micro-K for Liquid Suspension); Pataday (Olopatadine Hydrochloride Ophthalmic Solution); Patanase Nasal Spray (Olopatadine Hydrochloride Nasal Spray); PEG Electrolytes Solution (CoLyte); Pemirolast Potassium Ophthalmic Solution (Alamast); Penlac (Ciclopirox Topical Solution); PENNSAID (Diclofenac Sodium Topical Solution); Perforomist (Formoterol Fumarate Inhalation Solution); Peritoneal Dialysis Solution; Phenylephrine Hydrochloride Ophthalmic Solution (Neo-Synephrine); Phospholine Iodide (Echothiophate Iodide for Ophthalmic Solution); Podofilox (Podofilox Topical Solution); Pred Forte (Prednisolone Acetate Ophthalmic Suspension); Pralatrexate Solution for Intravenous Injection (Folotyn); Pred Mild; Prednisone Intensol; Prednisolone Acetate Ophthalmic Suspension (Pred Forte); Prevacid; PrismaSol Solution (Sterile Hemofiltration Hemodiafiltration Solution); ProAir; Proglycem; ProHance (Gadoteridol Injection Solution); Proparacaine Hydrochloride Ophthalmic Solution (Alcaine); Propine; Pulmicort; Pulmozyme; Quixin (Levofloxacin Ophthalmic Solution 0.5%); QVAR; Rapamune; Rebetol; Relacon-HC; Rotarix (Rotavirus Vaccine, Live, Oral Suspension); Rotavirus Vaccine, Live, Oral Suspension (Rotarix); Rowasa (Mesalamine Rectal Suspension Enema); Sabril (Vigabatrin Oral Solution); Sacrosidase Oral Solution (Sucraid); Sandimmune; Sepra; Serevent Diskus; Solu Cortef (Hydrocortisone Sodium Succinate); Solu Medrol (Methylprednisolone sodium succinate); Spiriva; Sporanox Oral Solution (Itraconazole Oral Solution); Staticin (Erythromycin Topical Solution 1.5%); Stalevo; Starlix; Sterile Hemofiltration Hemodiafiltration Solution (PrismaSol Solution); Stimate; Sucralfate (Carafate Suspension); Sulfacetamide Sodium Ophthalmic Solution 10% (Bleph 10); Synarel Nasal Solution (Nafarelin Acetate Nasal Solution for Endometriosis); Taclonex Scalp (Calcipotriene and Betamethasone Dipropionate Topical Suspension); Tamiflu; Tobi; TobraDex; Tobradex ST (Tobramycin/Dexamethasone Ophthalmic Suspension 0.3%/0.05%); Tobramycin/Dexamethasone Ophthalmic Suspension 0.3%/0.05% (Tobradex ST); Timolol; Timoptic; Travatan Z; Treprostinil Inhalation Solution (Tyvaso); Trusopt (Dorzolamide Hydrochloride Ophthalmic Solution); Tyvaso (Treprostinil Inhalation Solution); Ventolin; Vfend; Vibramycin Oral (Doxycycline Calcium Oral Suspension); Videx (Didanosine Pediatric Powder for Oral Solution); Vigabatrin Oral Solution (Sabril); Viokase; Viracept; Viramune; Vitamin K1 (Fluid Colloidal Solution of Vitamin K1); Voltaren Ophthalmic (Diclofenac Sodium Ophthalmic Solution); Zarontin Oral Solution (Ethosuximide Oral Solution); Ziagen; Zyvox; Zymar (Gatifloxacin Ophthalmic Solution); Zymaxid (Gatifloxacin Ophthalmic Solution).

### Drug Classes

5-alpha-reductaseinhibitors; 5-aminosalicylates; 5HT3 receptor antagonists; adamantane antivirals; adrenal cortical steroids; adrenal corticosteroid inhibitors; adrenergic bronchodilators; agents for hypertensive emergencies; agents for pulmonary hypertension; aldosterone receptor antagonists; alkylating agents; alpha-adrenoreceptor antagonists; alpha-glucosidase inhibitors; alternative medicines; amebicides; aminoglycosides; aminopenicillins; aminosalicylates; amylin analogs; Analgesic Combinations; Analgesics; androgens and anabolic steroids; angiotensin converting enzyme inhibitors; angiotensin II inhibitors; anorectal preparations; anorexiants; antacids; anthelmintics; anti-angiogenic ophthalmic agents; anti-CTLA-4 monoclonal antibodies; anti-infectives; antiadrenergic agents, centrally acting; antiadrenergic agents, peripherally acting; antiandrogens; antianginal agents; antiarrhythmic agents; antiasthmatic combinations; antibiotics/antineoplastics; anticholinergic antiemetics; anticholinergic antiparkinson agents; anticholinergic bronchodilators; anticholinergic chronotropic agents; anticholinergics/antispasmodics; anticoagulants; anticonvulsants; antidepressants; antidiabetic agents; antidiabetic combinations; antidiarrheals; antidiuretic hormones; antidotes; antiemetic/antivertigo agents; antifungals; antigonadotropic agents; antigout agents; antihistamines; antihyperlipidemic agents; antihyperlipidemic combinations; antihypertensive combinations; antihyperuricemic agents; antimalarial agents; antimalarial combinations; antimalarial quinolines; antimetabolites; antimigraine agents; antineoplastic detoxifying agents; antineoplastic interferons; antineoplastic monoclonal antibodies; antineoplastics; antiparkinson agents; antiplatelet agents; antipseudomonal penicillins; antipsoriatics; antipsychotics; antirheumatics; antiseptic and germicides; antithyroid agents; antitoxins and antivenins; antituberculosis agents; antituberculosis combinations; antitussives; antiviral agents; antiviral combinations; antiviral interferons; anxiolytics, sedatives, and hypnotics; aromatase inhibitors; atypical antipsychotics; azole antifungals; bacterial vaccines; barbiturate anticonvulsants; barbiturates; BCR-ABL tyrosine kinase inhibitors; benzodiazepine anticonvulsants; benzodiazepines; beta-adrenergic blocking agents; beta-lactamase inhibitors; bile acid sequestrants; biologicals; bisphosphonates; bone resorption inhibitors; bronchodilator combinations; bronchodilators; calcitonin; calcium channel blocking agents; carbamate anticonvulsants; carbapenems; carbonic anhydrase inhibitor anticonvulsants; carbonic anhydrase inhibitors; cardiac stressing agents; cardioselective beta blockers; cardiovascular agents; catecholamines; CD20 monoclonal antibodies; CD33 monoclonal antibodies; CD52 monoclonal antibodies; central nervous system agents; cephalosporins; cerumenolytics; chelating agents; chemokine receptor antagonist; chloride channel activators; cholesterol absorption inhibitors; cholinergic agonists; cholinergic muscle stimulants; cholinesterase inhibitors; CNS stimulants; coagulation modifiers; colony stimulating factors; contraceptives; corticotropin; coumarins and indandiones; cox-2 inhibitors; decongestants; dermatological agents; diagnostic radiopharmaceuticals; dibenzazepine anticonvulsants; digestive enzymes; dipeptidyl peptidase 4 inhibitors; diuretics; dopaminergic antiparkinsonism agents; drugs used in alcohol dependence; echinocandins; EGFR inhibitors; estrogen receptor antagonists; estrogens; expectorants; factor Xa inhibitors; fatty acid derivative anticonvulsants; fibric acid derivatives; first generation cephalosporins; fourth generation cephalosporins; functional bowel disorder agents; gallstone solubilizing agents; gamma-aminobutyric acid analogs; gamma-aminobutyric acid reuptake inhibitors; gamma-aminobutyric acid transaminase inhibitors; gastrointestinal agents; general anesthetics; genitourinary tract agents; GI stimulants; glucocorticoids; glucose elevating agents; glycopeptide antibiotics; glycoprotein platelet inhibitors; glycylcyclines; gonadotropin releasing hormones; gonadotropin-releasing hormone antagonists; gonadotropins; group I antiarrhythmics; group II antiarrhythmics; group III antiarrhythmics; group IV antiarrhythmics; group V antiarrhythmics; growth hormone receptor blockers; growth hormones; H. pylori eradication agents; H2 antagonists; hematopoietic stem cell mobilizer; heparin antagonists; heparins; HER2 inhibitors; herbal products; histone deacetylase inhibitors; hormone replacement therapy; hormones; hormones/antineoplastics; hydantoin anticonvulsants; illicit (street) drugs; immune globulins; immunologic agents; immunosuppressive agents; impotence agents; in vivo diagnostic biologicals; incretin mimetics; inhaled anti-infectives; inhaled corticosteroids; inotropic agents; insulin; insulin-like growth factor; integrase strand transfer inhibitor; interferons; intravenous nutritional products; iodinated contrast media; ionic iodinated contrast media; iron products; ketolides; laxatives; leprostatics; leukotriene modifiers; lincomycin derivatives; lipoglycopeptides; local injectable anesthetics; loop diuretics; lung surfactants; lymphatic staining agents; lysosomal enzymes; macrolide derivatives; macrolides; magnetic resonance imaging contrast media; mast cell stabilizers; medical gas; meglitinides; metabolic agents; methylxanthines; mineralocorticoids; minerals and electrolytes; miscellaneous agents; miscellaneous analgesics; miscellaneous antibiotics; miscellaneous anticonvulsants; miscellaneous antidepressants; miscellaneous antidiabetic agents; miscellaneous antiemetics; miscellaneous antifungals; miscellaneous antihyperlipidemic agents; miscellaneous antimalarials; miscellaneous antineoplastics; miscellaneous antiparkinson agents; miscellaneous antipsychotic agents; miscellaneous antituberculosis agents; miscellaneous antivirals; miscellaneous anxiolytics, sedatives and hypnotics; miscellaneous biologicals; miscellaneous bone resorption inhibitors; miscellaneous cardiovascular agents; miscellaneous central nervous system agents; miscellaneous coagulation modifiers; miscellaneous diuretics; miscellaneous genitourinary tract agents; miscellaneous GI agents; miscellaneous hormones; miscellaneous metabolic agents; miscellaneous ophthalmic agents; miscellaneous otic agents; miscellaneous respiratory agents; miscellaneous sex hormones; miscellaneous topical agents; miscellaneous uncategorized agents; miscellaneous vaginal agents; mitotic inhibitors; monoamine oxidase inhibitors; monoclonal antibodies; mouth and throat products; mTOR inhibitors; mTOR kinase inhibitors; mucolytics; multikinase inhibitors; muscle relaxants; mydriatics; narcotic analgesic combinations; narcotic analgesics; nasal anti-infectives; nasal antihistamines and decongestants; nasal lubricants and irrigations; nasal preparations; nasal steroids; natural penicillins; neuraminidase inhibitors; neuromuscular blocking agents; next generation cephalosporins; nicotinic acid derivatives; nitrates; NNRTIs; non-cardioselective beta blockers; non-iodinated contrast media; non-ionic iodinated contrast media; non-sulfonylureas; nonsteroidal anti-inflammatory agents; norepinephrine reuptake inhibitors; norepinephrine-dopamine reuptake inhibitors; nucleoside reverse transcriptase inhibitors (NRTIs); nutraceutical products; nutritional products; ophthalmic anesthetics; ophthalmic anti-infectives; ophthalmic anti-inflammatory agents; ophthalmic antihistamines and decongestants; ophthalmic diagnostic agents; ophthalmic glaucoma agents; ophthalmic lubricants and irrigations; ophthalmic preparations; ophthalmic steroids; ophthalmic steroids with anti-infectives; ophthalmic surgical agents; oral nutritional supplements; otic anesthetics; otic anti-infectives; otic preparations; otic steroids; otic steroids with anti-infectives; oxazolidinedione anticonvulsants; parathyroid hormone and analogs; penicillinase resistant penicillins; penicillins; peripheral opioid receptor antagonists; peripheral vasodilators; peripherally acting antiobesity agents; phenothiazine antiemetics; phenothiazine antipsychotics; phenylpiperazine antidepressants; plasma expanders; platelet aggregation inhibitors; platelet-stimulating agents; polyenes; potassium-sparing diuretics; probiotics; progesterone receptor modulators; progestins; prolactin inhibitors; prostaglandin D2 antagonists; protease inhibitors; proton pump inhibitors; psoralens; psychotherapeutic agents; psychotherapeutic combinations; purine nucleosides; pyrrolidine anticonvulsants; quinolones; radiocontrast agents; radiologic adjuncts; radiologic agents; radiologic conjugating agents; radiopharmaceuticals; RANK ligand inhibitors; recombinant human erythropoietins; renin inhibitors; respiratory agents; respiratory inhalant products; rifamycin derivatives; salicylates; sclerosing agents; second generation cephalosporins; selective estrogen receptor modulators; selective serotonin reuptake inhibitors; serotonin-norepinephrine reuptake inhibitors; serotoninergic neuroenteric modulators; sex hormone combinations; sex hormones; skeletal muscle relaxant combinations; skeletal muscle relaxants; smoking cessation agents; somatostatin and somatostatin analogs; spermicides; statins; sterile irrigating solutions; streptomyces derivatives; succinimide anticonvulsants; sulfonamides; sulfonylureas; synthetic ovulation stimulants; tetracyclic antidepressants; tetracyclines; therapeutic radiopharmaceuticals; thiazide diuretics; thiazolidinediones; thioxanthenes; third generation cephalosporins; thrombin inhibitors; thrombolytics; thyroid drugs; tocolytic agents; topical acne agents; topical agents; topical anesthetics; topical anti-infectives; topical antibiotics; topical antifungals; topical antihistamines; topical antipsoriatics; topical antivirals; topical astringents; topical debriding agents; topical depigmenting agents; topical emollients; topical keratolytics; topical steroids; topical steroids with anti-infectives; toxoids; triazine anticonvulsants; tricyclic antidepressants; trifunctional monoclonal antibodies; tumor necrosis factor (TNF) inhibitors; tyrosine kinase inhibitors; ultrasound contrast media; upper respiratory combinations; urea anticonvulsants; urinary anti-infectives; urinary antispasmodics; urinary pH modifiers; uterotonic agents; vaccine; vaccine combinations; vaginal anti-infectives; vaginal preparations; vasodilators; vasopressin antagonists; vasopressors; VEGF/VEGFR inhibitors; viral vaccines; viscosupplementation agents; vitamin and mineral combinations; vitamins.

### Diagnostic Tests

17-Hydroxyprogesterone; ACE (Angiotensin I converting enzyme); Acetaminophen; Acid phosphatase; ACTH; Activated clotting time; Activated protein C resistance; Adrenocorticotropic hormone (ACTH); Alanine aminotransferase (ALT); Albumin; Aldolase; Aldosterone; Alkaline phosphatase; Alkaline phosphatase (ALP); Alpha1-antitrypsin; Alpha-fetoprotein; Alpha-fetoprotien; Ammonia levels; Amylase; ANA (antinuclear antbodies); ANA (antinuclear antibodies); Angiotensin-converting enzyme (ACE); Anion gap; Anticardiolipin antibody; Anticardiolipin antivbodies (ACA); Anti-centromere antibody; Antidiuretic hormone; Anti-DNA; Anti-Dnase-B; Anti-Gliadin antibody; Anti-glomerular basement membrane antibody; Anti-HBc (Hepatitis B core antibodies; Anti-HBs (Hepatitis B surface antibody; Antiphospholipid antibody; Anti-RNA polymerase; Anti-Smith (Sm) antibodies; Anti-Smooth Muscle antibody; Antistreptolysin O (ASO); Antithrombin III; Anti-Xa activity; Anti-Xa assay; Apolipoproteins; Arsenic; Aspartate aminotransferase (AST); B12; Basophil; Beta-2-Microglobulin; Beta-hydroxybutyrate; B-HCG; Bilirubin; Bilirubin, direct; Bilirubin, indirect; Bilirubin, total; Bleeding time; Blood gases (arterial); Blood urea nitrogen (BUN); BUN; BUN (blood urea nitrogen); CA 125; CA 15-3; CA 19-9; Calcitonin; Calcium; Calcium (ionized); Carbon monoxide (CO); Carcinoembryonic antigen (CEA); CBC; CEA; CEA (carcinoembryonic antigen); Ceruloplasmin; CH50OChloride; Cholesterol; Cholesterol, HDL; Clot lysis time; Clot retraction time; CMP; CO2; Cold agglutinins; Complement C3; Copper; Corticotrophin releasing hormone (CRH) stimulation test; Cortisol; Cortrosyn stimulation test; C-peptide; CPK (Total); CPK-MB; C-reactive protein; Creatinine; Creatinine kinase (CK); Cryoglobulins; DAT (Direct antiglobulin test); D-Dimer; Dexamethasone suppression test; DHEA-S; Dilute Russell viper venom; Elliptocytes; Eosinophil; Erythrocyte sedimentation rate (ESR); Estradiol; Estriol; Ethanol; Ethylene glycol; Euglobulin lysis; Factor V Leiden; Factor VIII inhibitor; Factor VIII level; Ferritin; Fibrin split products; Fibrinogen; Folate; Folate (serum; Fractional excretion of sodium (FENA); FSH (follicle stimulating factor); FTA-ABS; Gamma glutamyl transferase (GGT); Gastrin; GGTP (Gamma glutamyl transferase); Glucose; Growth hormone; Haptoglobin; HBeAg (Hepatitis Be antigen); HBs-Ag (Hepatitis B surface antigen); Helicobacter pylori; Hematocrit; Hematocrit (HCT); Hemoglobin; Hemoglobin AlC; Hemoglobin electrophoresis; Hepatitis A antibodies; Hepatitis C antibodies; IAT (Indirect antiglobulin test); Immunofixation (IFE); Iron; Lactate dehydrogenase (LDH); Lactic acid (lactate); LDH; LH (Leutinizing hormone; Lipase; Lupus anticoagulant; Lymphocyte; Magnesium; MCH (mean corpuscular hemoglobin; MCHC (mean corpuscular hemoglobin concentration); MCV (mean corpuscular volume); Methylmalonate; Monocyte; MPV (mean platelet volume); Myoglobin; Neutrophil; Parathyroid hormone (PTH); Phosphorus; Platelets (plt); Potassium; Prealbumin; Prolactin; Prostate specific antigen (PSA); Protein C; Protein S; PSA (prostate specific antigen); PT (Prothrombin time); PTT (Partial thromboplastin time); RDW (red cell distribution width); Renin; Rennin; Reticulocyte count; reticulocytes; Rheumatoid factor (RF); Sed Rate; Serum glutamic-pyruvic transaminase (SGPT; Serum protein electrophoresis (SPEP); Sodium; T3-resin uptake (T3RU); T4, Free; Thrombin time; Thyroid stimulating hormone (TSH); Thyroxine (T4); Total iron binding capacity (TIBC); Total protein; Transferrin; Transferrin saturation; Triglyceride (TG); Troponin; Uric acid; Vitamin B12; White blood cells (WBC); Widal test.

### BRIEF DESCRIPTION OF THE DRAWINGS

FIG. 1 illustrates an exploded perspective view of a container according to the present disclosure, with the flexible bag 18 partially cut away to illustrate its interior.

FIG. 2 illustrates an axial sectional view of an apparatus for applying a PECVD SiOx coating on a two-dimensional flexible polymer film roll, wherein the film, in subsequent processing steps is severable into sections and wherein one or more sections may be combined to form a storage bag that may be used according to the present disclosure.

FIG. 3 illustrates an axial sectional view of an alternative apparatus for applying a PECVD SiOx coating on a two-dimensional flexible polymer film roll, wherein the film, in subsequent processing steps is severable into sections and wherein one or more sections may be combined to form a storage bag that may be used according to the present disclosure.

FIG. 4 illustrates a fragmentary section taken along section line a-a in FIG. 1 or FIG. 7 of a face-to-face seal according to any embodiment of this disclosure.

FIGS. 5 and 6 illustrate fragmentary sections taken along section line a-a in FIG. 1 or FIG. 7 of a lapped seal according to any embodiment of this disclosure.

FIG. 7 illustrates a plan view of a flexible bag 18 having an alternative seal plan, which can be substituted in FIG. 1 in any embodiment, with the flexible bag 18 partially cut away to illustrate its interior.

FIG. 8 illustrates a plan view of a flexible bag 18 having three spouts 24 for introduction of material from two or more sources and for removal of a reaction product.

The following reference characters are used in the drawings figures:

FIG. 9 is a schematic view of a chemical vapor deposition coating system useful for application of the coatings or layers of the present disclosure.

FIG. 10 is a Fourier Transform Infrared Spectrophotometer (FTIR) absorbance spectrum of a PECVD coating.

FIG. 11 is a Fourier Transform Infrared Spectrophotometer (FTIR) absorbance spectrum of a PECVD coating.

FIG. 12 is a Fourier Transform Infrared Spectrophotometer (FTIR) absorbance spectrum of a PECVD coating.

FIG. 13 is a Fourier Transform Infrared Spectrophotometer (FTIR) absorbance spectrum of a PECVD coating.

FIG. 14 is a Fourier Transform Infrared Spectrophotometer (FTIR) absorbance spectrum of a PECVD coating.

FIG. 15 is a Fourier Transform Infrared Spectrophotometer (FTIR) absorbance spectrum of a PECVD coating.

FIG. 16 is a Fourier Transform Infrared Spectrophotometer (FTIR) absorbance spectrum of a PECVD coating.

FIG. 17 is a Fourier Transform Infrared Spectrophotometer (FTIR) absorbance spectrum of a PECVD coating.

FIG. 18 is a Fourier Transform Infrared Spectrophotometer (FTIR) absorbance spectrum of a PECVD coating.

FIG. 19 is a Fourier Transform Infrared Spectrophotometer (FTIR) absorbance spectrum of a PECVD coating.

FIG. 20 is a Fourier Transform Infrared Spectrophotometer (FTIR) absorbance spectrum of a PECVD coating.

FIG. 21 is a Fourier Transform Infrared Spectrophotometer (FTIR) absorbance spectrum of a PECVD coating.

FIG. 22 is a Fourier Transform Infrared Spectrophotometer (FTIR) absorbance spectrum of a PECVD coating.

FIG. 23 is a Fourier Transform Infrared Spectrophotometer (FTIR) absorbance spectrum of a PECVD coating.

FIG. 24 is a Fourier Transform Infrared Spectrophotometer (FTIR) absorbance spectrum of a PECVD coating.

FIG. 25 is a Fourier Transform Infrared Spectrophotometer (FTIR) absorbance spectrum of a PECVD coating.

FIG. 26 is a Fourier Transform Infrared Spectrophotometer (FTIR) absorbance spectrum of a PECVD coating.

FIG. 27 is a Fourier Transform Infrared Spectrophotometer (FTIR) absorbance spectrum of a PECVD coating.

FIG. 28 is a schematic view of one of the systems for coating the vessels.

FIG. 29 is an image of an inverted i-chem jar during incubation in Example 1.

FIG. 30 presents LC-MS spectra of the extractables from the uncoated film (top scheme) and pH protective coating coated film (bottom scheme).

FIG. 31 presents LC-MS spectra of the extractables from stretched/elongated films coated with protective coating.

FIG. 32 presents the SEM images of the protective coating coated films after being stretched/elongated by 0%, 20%, 30% and 40%.

FIG. 33 presents the SEM images of the barrier coating coated films after being stretched/elongated by 0%, 5%, 10%, 50% and 100%.

FIG. 34 presents LC-MS spectra of the extractables from the trilayer coated films after being stretched/elongated by 0%, 10%, 25%, 50% and 100% except that the top scheme is the LC-MS spectra of the extractables from uncoated film as a reference.

FIG. 35 is a schematic sectional view of a coated vessel according to an embodiment of the disclosure.

FIG. 36 is an enlarged sectional view of the inner surface of a pH protective coating coated vessel of FIG. 1 according to an embodiment.

FIG. 37 is an enlarged sectional view of the inner surface of a trilayer coating coated vessel of FIG. 1 according to an embodiment.

FIG. 38 is an enlarged sectional view of the inner surface of a SiOx coating coated vessel of FIG. 1 according to an embodiment.

FIG. 39 is an image of an exemplary rigid frame in which the coated package is placed according to one embodiment.

FIG. 40 is an image of an exemplary flexible intermediate bulk container (FIBC) in which the coated package is placed according to one embodiment.

The following reference characters are used in the drawings figures:

| | |
|---|---|
| 10 | Container |
| 18 | Flexible bag |
| 20 | Film sheet |
| 22 | Seal |
| 24 | Spout |
| 28 | End seal |
| 30 | Barrier coating |
| 32 | Surface portion |
| 34 | Lapped seal |
| 36 | Face-to-face seal |
| 38 | Side seal |
| 40 | Perimeter seal |
| 42 | Valve (of 24) |
| 44 | Solvent |
| 46 | Lumen |
| 48 | Fused portion |
| 50 | Vessel holder |
| 98 | Vacuum source |
| 100 | PECVD apparatus |
| 102 | Polymer film |
| 104 | Unwind reel |
| 106 | Quick roller |
| 108 | Guide roller |
| 110 | Rewind reel |
| 112 | Chamber |
| 114 | Treatment area |
| 116 | Diffusion pump |
| 118 | Gas inlet |
| 120 | Unbalanced magnetron |
| 122 | Plasma energy source |
| 124 | Cathode |
| 144 | Reactant gas source |
| 152 | Pressure gauge |
| 160 | Outer electrode |
| 162 | Power supply |
| 168 | Top end |
| 202 | Polymer film |
| 210 | Vessel |
| 212 | Lumen |
| 214 | Wall |
| 216 | Outer surface |
| 218 | Fluid |
| 258 | Plunger |
| 285 | Coatings |
| 286 | pH protective coating or layer |
| 288 | Barrier layer |
| 289 | Tie coating or layer |
| 302 | Tie coater |
| 304 | Barrier coater |
| 306 | Protective coater |
| 308 | Fluid filler |
| 310 | Fluid supply |
| 312 | Closure installer |
| 314 | Closure supply |
| 404 | Vent |
| 574 | Main vacuum valve |
| 576 | Vacuum line |
| 578 | Manual bypass valve |
| 580 | Bypass line |
| 582 | Vent valve |
| 584 | Main reactant gas valve |
| 586 | Main reactant feed line |
| 588 | Reservoir |
| 590 | Capillary line |
| 592 | Shut off valve |
| 594 | Oxygen tank |
| 596 | Oxygen feed line |
| 598 | Mass flow controller |
| 600 | Oxygen shut-off valve |
| 602 | Reservoir |
| 604 | Feed line |
| 606 | Shut-off valve |
| 614 | Headspace |
| 616 | Pressure source |
| 618 | Pressure valve |
| 620 | Capillary connector |

### Definitions

In the context of the present disclosure, the following definitions and abbreviations are used.

RF is radio frequency.

The term “at least” in the context of the present disclosure means “equal or more” than the integer following the term. The word “comprising” does not exclude other elements or steps, and the indefinite article “a” or “an” does not exclude a plurality unless indicated otherwise. Whenever a parameter range is indicated, it is intended to disclose the parameter values given as limits of the range and all values of the parameter falling within said range.

“First” and “second” or similar references to, for example, processing stations or processing devices refer to the minimum number of processing stations or devices that are present, but do not necessarily represent the order or total number of processing stations and devices. These terms do not limit the number of processing stations or the particular processing carried out at the respective stations.

For purposes of the present disclosure, an “organosilicon precursor” is a compound having at least one of the linkages:

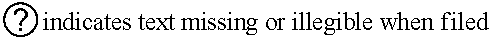

which is a tetravalent silicon atom connected to an oxygen or nitrogen atom and an organic carbon atom (an organic carbon atom being a carbon atom bonded to at least one hydrogen atom). A volatile organosilicon precursor, defined as such a precursor that can be supplied as a vapor in a PECVD apparatus, can be an optional organosilicon precursor. Optionally, the organosilicon precursor can be selected from the group consisting of a linear siloxane, a monocyclic siloxane, a polycyclic siloxane, a polysilsesquioxane, an alkyl trimethoxysilane, a linear silazane, a monocyclic silazane, a polycyclic silazane, a polysilsesquiazane, and a combination of any two or more of these precursors.

The feed amounts of PECVD precursors, gaseous reactant or process gases, and carrier gas are sometimes expressed in “standard volumes” in the specification and claims. The standard volume of a charge or other fixed amount of gas is the volume the fixed amount of the gas would occupy at a standard temperature and pressure (without regard to the actual temperature and pressure of delivery). Standard volumes can be measured using different units of volume, and still be within the scope of the present disclosure and claims. For example, the same fixed amount of gas could be expressed as the number of standard cubic centimeters, the number of standard cubic meters, or the number of standard cubic feet. Standard volumes can also be defined using different standard temperatures and pressures, and still be within the scope of the present disclosure and claims. For example, the standard temperature might be 0° C. and the standard pressure might be 760 Torr (as is conventional), or the standard temperature might be 20° C. and the standard pressure might be 1 Torr. But whatever standard is used in a given case, when comparing relative amounts of two or more different gases without specifying particular parameters, the same units of volume, standard temperature, and standard pressure are to be used relative to each gas, unless otherwise indicated.

The corresponding feed rates of PECVD precursors, gaseous reactant or process gases, and carrier gas are expressed in standard volumes per unit of time in the specification. For example, in the working examples the flow rates are expressed as standard cubic centimeters per minute, abbreviated as sccm. As with the other parameters, other units of time can be used, such as seconds or hours, but consistent parameters are to be used when comparing the flow rates of two or more gases, unless otherwise indicated.

A “vessel” in the context of the present disclosure can be any type of article with at least one opening and a wall defining an inner or interior surface. The substrate can be the inside wall of a vessel having a lumen. Though the disclosure is not necessarily limited to pharmaceutical packages or other vessels of a particular volume, pharmaceutical packages or other vessels are contemplated in which the lumen can have a void volume of from 0.001 mL to 1000 mL, optionally 0.5 to 50 mL, optionally from 1 to 10 mL, optionally from 0.5 to 5 mL, optionally from 1 to 3 mL. The substrate surface can be part or all of the inner or interior surface inner or interior surface of a vessel having at least one opening and an inner or interior surface inner or interior surface.

A vessel in the context of the present disclosure can have one or more openings. One or two openings, like the openings of a sample tube (one opening) or a syringe barrel (two openings) are preferred. If the vessel has two openings, they can be the same size or different sizes. If there is more than one opening, one opening can be used for the gas inlet for a PECVD coating method according to the present disclosure, while the other openings are either capped or open. A vessel according to the present disclosure can be a sample tube, for example for collecting or storing biological fluids like blood or urine, a syringe (or a part thereof, for example a syringe barrel) for storing or delivering a biologically active compound or composition, for example a medicament or pharmaceutical composition, a vial for storing biological materials or biologically active compounds or compositions, a pipe, for example a catheter for transporting biological materials or biologically active compounds or compositions, or a cuvette for holding fluids, for example for holding biological materials or biologically active compounds or compositions.

The vessel can be provided with a reagent or preservative for sample collection or analysis. For example, a vessel for blood collection can have an inner or interior surface defining a lumen and an exterior surface, the passivation layer or pH protective coating can be on the inner or interior surface, and the vessel can contain a compound or composition in its lumen, for example citrate or a citrate containing composition.

A vessel can be of any shape, a vessel having a substantially cylindrical wall adjacent to at least one of its open ends being preferred. Generally, the interior wall of the vessel can be cylindrically shaped, like, for example in a sample tube or a syringe barrel. Sample tubes and syringes or their parts (for example syringe barrels) are contemplated.

A “hydrophobic layer” in the context of the present disclosure means that the coating or layer lowers the wetting tension of a surface coated with the coating or layer, compared to the corresponding uncoated surface. Hydrophobicity can be thus a function of both the uncoated substrate and the coating or layer. The same applies with appropriate alterations for other contexts wherein the term “hydrophobic” is used. The term “hydrophilic” means the opposite, i.e. that the wetting tension is increased compared to reference sample. The present hydrophobic layers are primarily defined by their hydrophobicity and the process conditions providing hydrophobicity. Suitable hydrophobic coatings or layers and their application, properties, and use are described in U.S. Pat. No. 7,985,188, which is incorporated by reference in its entirety herein for all purposes. Additional coatings of applicability are disclosed in U.S. Pat. No. 9,554,968, PCTUS2014023813, PCTUS2015022154, PCTUS2012064489, U.S. Ser. No. 14/357,418, PCTUS2014023813, U.S. Ser. No. 14/774,073, PCTUS1348709, U.S. Ser. No. 14/412,472, PCTUS2016047622, and/or U.S. Ser. No. 13/240,797, each of which is incorporated by reference in its entirety herein for all purposes. Dual functional passivation layers or pH protective coatings that also have the properties of hydrophobic coatings or layers can be provided for any embodiment of the present disclosure.

The values of w, x, y, and z are applicable to the empirical composition SiwOxCyHz throughout this specification. The values of w, x, y, and z used throughout this specification should be understood as ratios or an empirical formula (for example for a coating or layer), rather than as a limit on the number or type of atoms in a molecule. For example, octamethylcyclotetrasiloxane, which has the molecular composition Si4O4C8H24, can be described by the following empirical formula, arrived at by dividing each of w, x, y, and z in the molecular formula by 4, the largest common factor: Si1O1C2H6. The values of w, x, y, and z are also not limited to integers. For example, (acyclic) octamethyltrisiloxane, molecular composition Si3O2C8H24, is reducible to Si1O0.67C2.67H8. Also, although SiOxCyHz can be described as equivalent to SiOxCy, it is not necessary to show the presence of hydrogen in any proportion to show the presence of SiOxCy.

“Wetting tension” is a specific measure for the hydrophobicity or hydrophilicity of a surface. An optional wetting tension measurement method in the context of the present disclosure is ASTM D 2578 or a modification of the method described in ASTM D 2578. This method uses standard wetting tension solutions (called dyne solutions) to determine the solution that comes nearest to wetting a plastic film surface for exactly two seconds. This is the film's wetting tension. The procedure utilized can be varied herein from ASTM D 2578 in that the substrates are not flat plastic films, but are tubes made according to the Protocol for Forming PET Tube and (except for controls) coated according to the Protocol for coating Tube Interior with Hydrophobic Coating or Layer (see Example 9 of EP2251671 A2).

A “lubricity coating or layer” according to the present disclosure is a coating or layer which has a lower frictional resistance than the uncoated surface.

A “passivation layer or pH protective coating” according to the present disclosure passivates or protects an underlying surface or layer from a fluid composition contacting the layer (as more extensively defined elsewhere in this specification).

Coatings of SiOx are deposited by plasma enhanced chemical vapor deposition (PECVD) or other chemical vapor deposition processes on the vessel of a pharmaceutical package, in particular a thermoplastic package, to serve as a barrier coating or layer preventing oxygen, air, carbon dioxide, or other gases from entering the vessel and/or to prevent leaching of the pharmaceutical material into or through the package wall. The barrier coating or layer can be effective to reduce the ingress of atmospheric gas, for example oxygen, into the lumen compared to a vessel without a passivation layer or pH protective coating.

In any embodiment the vapor-deposited coating or layer optionally can also, or alternatively, be a solute barrier coating or layer. A concern of converting from glass to plastic syringes centers around the potential for leachable materials from plastics. With plasma coating technology, the coatings or layers derived from non-metal gaseous precursors, for example HMDSO or OMCTS or other organosilicon compounds, will contain no trace metals and function as a barrier coating or layer to inorganic, metals and organic solutes, preventing leaching of these species from the coated substrate into syringe fluids. In addition to leaching control of plastic syringes, the same plasma passivation layer or pH protective coating technology offers potential to provide a solute barrier to the plunger tip, piston, stopper, or seal, typically made of elastomeric plastic compositions containing even higher levels of leachable organic oligomers and catalysts.

Moreover, certain syringes prefilled with synthetic and biological pharmaceutical formulations are very oxygen and moisture sensitive. A critical factor in the conversion from glass to plastic syringe barrels will be the improvement of plastic oxygen and moisture barrier performance. The plasma passivation layer or pH protective coating technology can be suitable to maintain the SiOx barrier coating or layer or layer for protection against oxygen and moisture over an extended shelf life.

Examples of solutes in drugs usefully excluded by a barrier layer in any embodiment include antibacterial preservatives, antioxidants, chelating agents, pH buffers, and combinations of any of these. In any embodiment the vapor-deposited coating or layer optionally can be a solvent barrier coating or layer for a solvent comprising a co-solvent used to increase drug solubilization.

In any embodiment the vapor-deposited coating or layer optionally can be a barrier coating or layer for water, glycerin, propylene glycol, methanol, ethanol, n-propanol, isopropanol, acetone, benzyl alcohol, polyethylene glycol, cotton seed oil, benzene, dioxane, or combinations of any two or more of these.

In any embodiment the vapor-deposited coating or layer optionally can be a metal ion barrier coating or layer.

In any embodiment the vapor-deposited coating or layer optionally can be a barrel wall material barrier coating or layer, to prevent or reduce the leaching of barrel material such as any of the base barrel resins mentioned previously and any other ingredients in their respective compositions.

The inventors have found, however, that such barrier coatings or layers or coatings of SiOx are eroded or dissolved by some fluid compositions, for example aqueous compositions having a pH above about 5. Since coatings applied by chemical vapor deposition can be very thin—tens to hundreds of nanometers thick—even a relatively slow rate of erosion can remove or reduce the effectiveness of the barrier coating or layer in less time than the desired shelf life of a product package. This can be particularly a problem for fluid pharmaceutical compositions, since many of them have a pH of roughly 7, or more broadly in the range of 5 to 9, similar to the pH of blood and other human or animal fluids. The higher the pH of the pharmaceutical preparation, the more quickly it erodes or dissolves the SiOx coating.

The inventors have further found that without a protective coating borosilicate glass surfaces are eroded or dissolved by some fluid compositions, for example aqueous compositions having a pH above about 5. This can be particularly a problem for fluid pharmaceutical compositions, since many of them have a pH of roughly 7, or more broadly in the range of 5 to 9, similar to the pH of blood and other human or animal fluids. The higher the pH of the pharmaceutical preparation, the more quickly it erodes or dissolves the glass. Delamination of the glass can also result from such erosion or dissolution, as small particles of glass are undercut by the aqueous compositions having a pH above about 5.

The inventors have further found that certain passivation layers or pH protective coatings of SiOxCy or SiNxCy formed from cyclic polysiloxane precursors, which passivation layers or pH protective coatings have a substantial organic component, do not erode quickly when exposed to fluid compositions, and in fact erode or dissolve more slowly when the fluid compositions have higher pHs within the range of 5 to 9. For example, at pH 8, the dissolution rate of a passivation layer or pH protective coating made from the precursor octamethylcyclotetrasiloxane, or OMCTS, can be quite slow. These passivation layers or pH protective coatings of SiOxCy or SiNxCy can therefore be used to cover a barrier coating or layer of SiOx, retaining the benefits of the barrier coating or layer by passivating or protecting it from the fluid composition in the pharmaceutical package. These passivation layers or pH protective coatings of SiOxCy or SiNxCy also can be used to cover a glass surface, for example a borosilicate glass surface, preventing delamination, erosion and dissolution of the glass, by passivating or protecting it from the fluid composition in the pharmaceutical package.

Although the present disclosure does not depend upon the accuracy of the following theory, it is believed that the material properties of an effective SiOxCy passivation layer or pH protective coating and those of an effective lubricity layer as described in U.S. Pat. No. 7,985,188 and in International Application PCT/US11/36097 are similar in some instances, such that a coating having the characteristics of a lubricity layer as described in certain working examples of this specification, U.S. Pat. No. 7,985,188, or International Application PCT/US11/36097 will also in certain cases serve as well as a passivation layer or pH protective coating to passivate or protect the barrier coating or layer of the package and vice versa.

Other precursors and methods can be used to apply the pH protective coating or layer or passivating treatment. Similarly, these can be used as a separate surface coatings or layers in addition to or as an alternative to the pH protective coatings or layers described above. To accommodate the latter format, these layers and coatings are referred to herein as surface layers and coatings but may be described herein as a passivation or pH protective treatment. For example, hexamethylene disilazane (HMDZ) can be used as the precursor. HMDZ has the advantage of containing no oxygen in its molecular structure. This passivation treatment is contemplated to be a surface treatment of the SiOx barrier layer with HMDZ. To slow down and/or eliminate the decomposition of the silicon dioxide coatings at silanol bonding sites, the coating must be passivated. It is contemplated that passivation of the surface with HMDZ (and optionally application of a few mono layers of the HMDZ-derived coating) will result in a toughening of the surface against dissolution, resulting in reduced decomposition. It is contemplated that HMDZ will react with the —OH sites that are present in the silicon dioxide coating, resulting in the evolution of NH3 and bonding of S—(CH3)3 to the silicon (it is contemplated that hydrogen atoms will be evolved and bond with nitrogen from the HMDZ to produce NH3).

It is contemplated that this HMDZ passivation can be accomplished through several possible paths.

One contemplated path is dehydration/vaporization of the HMDZ at ambient temperature. First, an SiOx surface is deposited, for example using hexamethylene disiloxane (HNDSO). The as-coated silicon dioxide surface is then reacted with HNDZ vapor. In an embodiment, as soon as the SiOx surface is deposited onto the article of interest, the vacuum is maintained. The HMDSO and oxygen are pumped away and a base vacuum is achieved. Once base vacuum is achieved, HMDZ vapor is flowed over the surface of the silicon dioxide (as coated on the part of interest) at pressures from the mTorr range to many Torr. The HMDZ is then pumped away (with the resulting NH3 that is a by-product of the reaction). The amount of NH3 in the gas stream can be monitored (with a residual gas analyzer—RGA—as an example) and when there is no more NH3 detected, the reaction is complete. The part is then vented to atmosphere (with a clean dry gas or nitrogen). The resulting surface is then found to have been passivated. It is contemplated that this method optionally can be accomplished without forming a plasma.

Alternatively, after formation of the SiOx barrier coating or layer, the vacuum can be broken before dehydration/vaporization of the HMDZ. Dehydration/vaporization of the HMDZ can then be carried out in either the same apparatus used for formation of the SiOx barrier coating or layer or different apparatus.

Dehydration/vaporization of HMDZ at an elevated temperature is also contemplated. The above process can alternatively be carried out at an elevated temperature exceeding room temperature up to about 150° C. The maximum temperature is determined by the material from which the coated part is constructed. An upper temperature should be selected that will not distort or otherwise damage the part being coated.

Dehydration/vaporization of HMDZ with a plasma assist is also contemplated. After carrying out any of the above embodiments of dehydration/vaporization, once the HMDZ vapor is admitted into the part, a plasma is generated. The plasma power can range from a few watts to 100+ watts (similar powers as used to deposit the SiOx). The above is not limited to HMDZ and could be applicable to any molecule that will react with hydrogen, for example any of the nitrogen-containing precursors described in this specification.

Another way of applying the pH protective coating or layer is to apply as the pH protective coating or layer an amorphous carbon or fluorocarbon coating (or a fluorinated hydrocarbon coating), or a combination of the two.

Amorphous carbon coatings can be formed by PECVD using a saturated hydrocarbon, (e.g. methane or propane) or an unsaturated hydrocarbon (e.g. ethylene, acetylene) as a precursor for plasma polymerization. Fluorocarbon coatings (or a fluorinated hydrocarbon coating) can be derived from fluorocarbons (for example, hexafluoroethylene or tetrafluoroethylene). Either type of coating, or a combination of both, can be deposited by vacuum PECVD or atmospheric pressure PECVD.

It is further contemplated that fluorosilicon precursors can be used to provide a pH protective coating or layer over an SiOx barrier layer. This can be carried out by using as a precursor a fluorinated silane precursor such as hexafluorosilane and a PECVD process. The resulting coating would also be expected to be a non-wetting coating.

It is further contemplated that any embodiment of the pH protective coating or layer processes described in this specification can also be carried out without using the article to be coated to contain the plasma.

Yet another coating modality contemplated for protecting or passivating an SiOx barrier layer is coating the barrier layer using a polyamidoamine epichlorohydrin resin. For example, the barrier coated part can be dip coated in a fluid polyamidoamine epichlorohydrin resin melt, solution or dispersion and cured by autoclaving or other heating at a temperature between 60 and 100° C. It is contemplated that a coating of polyamidoamine epichlorohydrin resin can be preferentially used in aqueous environments between pH 5-8, as such resins are known to provide high wet strength in paper in that pH range. Wet strength is the ability to maintain mechanical strength of paper subjected to complete water soaking for extended periods of time, so it is contemplated that a coating of polyamidoamine epichlorohydrin resin on an SiOx barrier layer will have similar resistance to dissolution in aqueous media. It is also contemplated that, because polyamidoamine epichlorohydrin resin imparts a lubricity improvement to paper, it will also provide lubricity in the form of a coating on a thermoplastic surface made of, for example, COC or COP.

Even another approach for protecting an SiOx layer is to apply as a pH protective coating or layer a liquid-applied coating of a polyfluoroalkyl ether, followed by atmospheric plasma curing the pH protective coating or layer. For example, it is contemplated that the process practiced under the trademark TriboGlide®, described in this specification, can be used to provide a pH protective coating or layer that is also a lubricity layer, as TriboGlide® is conventionally used to provide lubricity.

The surface layers and coatings, and the pH protection or passivation coatings and layers, are described herein as protecting an SiOx layer or coating; but that is not required for the embodiments of the present disclosure. The surface layers and coatings, and the pH protection or passivation coatings and layers, may be applied directly to a surface of the wall of the vessel or container or other surface, such as a film or bag.

The preferred drug contact surface includes a coating or layer that provides flexibility while retaining the desirable characteristics of the coatings or layers described herein, including but not limited to moisture barrier, resistance to degradation, compatibility, and the like. Of particular interest is a coating or layer that can provide 1×, 10×, 100×, or larger stretch and elongation of the underlying surface, wall, or film, without detrimentally reducing the desirable characteristics of the coatings or layers described herein, including but not limited to moisture barrier, resistance to degradation, compatibility, and the like. Accordingly, while the embodiments of the present disclosure provide one or more such coatings and layers, other coatings and layers may be contemplated within the scope and breadth of the current disclosure.

### DETAILED DESCRIPTION

The present disclosure will now be described more fully, with reference to the accompanying drawings, in which several embodiments are shown. This disclosure can, however, be embodied in many different forms and should not be construed as limited to the embodiments set forth here. Rather, these embodiments are examples of the disclosure, which has the full scope indicated by the language of the claims. Like numbers refer to like or corresponding elements throughout. The following disclosure relates to all embodiments unless specifically limited to a certain embodiment.

### CAR T-cell Therapy

CAR T-cell therapy: A type of treatment in which a patient's T cells (a type of immune cell) are changed in the laboratory (or pharmaceutical plant) so they will bind to cancer cells and kill them. Blood from a vein in the patient's arm flows through a tube to an apheresis machine, which removes the white blood cells, including the T cells, and sends the rest of the blood back to the patient. Then, the gene for a special receptor called a chimeric antigen receptor (CAR) is inserted into the T cells in the laboratory (or pharmaceutical plant). Millions of the CAR T cells are grown in the laboratory (or pharmaceutical plant) and then given to the patient by infusion. The CAR T cells are able to bind to an antigen on the cancer cells and kill them.

### CAR-T Outline

The typical CAR T cell manufacturing process begins with harvesting the patient's peripheral blood mononuclear cells (PBMCs) through leukapheresis. Leukapheresis is a procedure to separate and collect white blood cells. It is the first step in a treatment called CAR (chimeric antigen receptor) T-cell therapy. The collected T-cells are used to make a special version of T-cells called CARs. Leukapheresis typically occurs over several hours, during which the patient's blood is treated with anticoagulants and centrifuged to remove excess red blood cells and platelets. A peripheral blood mononuclear cell (PBMC) is any peripheral blood cell having a round nucleus. These cells consist of lymphocytes (T cells, B cells, NK cells) and monocytes, whereas erythrocytes and platelets have no nuclei, and granulocytes (neutrophils, basophils, and eosinophils) have multi-lobed nuclei. In humans, lymphocytes make up the majority of the PBMC population, followed by monocytes. Apheresis is a medical technology in which the blood of a person is passed through an apparatus that separates out one particular constituent and returns the remainder to the circulation. It is thus an extracorporeal therapy. Bioengineering solutions can be used to improve leukapheresis from an extended outpatient procedure to a process that substitutes implantable devices for traditional blood filtration. For instance, subcutaneous biomaterial scaffolds have been developed to recruit specific T cell subsets in vivo. Additionally, functionalized carbon nanotubes have been shown to successfully recruit and activate T cells in vitro and similar approaches could potentially be used in vivo. Within this model, the device would be implanted into the patient under a sterile field to reduce the probability of infection, and harvested a few days later with an enriched population of cytotoxic T cells suitable for transfection. Hematologic malignancies are forms of cancer that begin in the cells of blood-forming tissue, such as the bone marrow, or in the cells of the immune system. Examples of hematologic cancer are acute and chronic leukemias, lymphomas, multiple myeloma and myelodysplastic syndromes. CAR-T showing similar adoption presently as bone marrow transplant (BMT) when first started. Cytokine release syndrome is a form of systemic inflammatory response syndrome that arises as a complication of some diseases or infections and is also an adverse effect of some monoclonal antibody drugs, as well as adoptive T-cell therapies. Neurotoxicity is a form of toxicity in which a biological, chemical, or physical agent produces an adverse effect on the structure or function of the central and/or peripheral nervous system. It has proved challenging to find proper target antigens for solid tumors, and strategies to improve T cell penetration into the tumor microenvironment are needed. CAR-T currently most effective treating blood based cancers. Autologous stem-cell transplantation in which stem cells (undifferentiated cells from which other cell types develop) are removed from a person, stored, and later given back to that same person. Although some current clinical trials have successfully used freezing and thawing to transport T cells, remains room for improvement:

a. QC mechanisms to confirm cell viability and immune profile changes
b. Removal of DMSO—cryopreservation reagents.
c. Uses of hypothermic preservation solutions to eliminate the need to freeze the blood.

Activation—The most commonly used activation process is independent of antigen presentation and involves culturing T cells with beads coated with CD3/CD28 antibody fragments, along with IL-2 supplementation. The current method is time consuming and sustained signaling (activation) can cause exhaustion.

Alternative Activation Method—Tissue engineering approaches may improve the activation process via customizable ligand-presenting scaffolds in the place of aAPCs. These could feature controlled spatial or temporal patterns of ligand presentation.

T-cell Expansion—Expansion is required to increase the population of T cells available for transduction or infusion to the patient and can occur either before or after gene transduction, depending on the manufacturer. Use a single use bioprocessing bag—wave bag, rocking bags, etc.

The cell expansion process takes approximately ten days, upon which cells are harvested and cryopreserved for distribution. Problems with beads are aggregation especially when agitated in a bioprocessing bag. Removing the beads at the end of the process can cause shear stress thus damaging the T-cells.

Gene Transfers—Introduction of viral vectors to the T-cells. As this is a limiting factor in the overall efficacy of CAR T cell therapies, bioengineering strategies to improve gene transfer are in high demand.

The predominant safety concerns for therapies currently in the clinic are cytokine release syndrome, neurotoxicities and off-target CAR T cell activity, all of which have resulted in severe adverse events, and in some cases, patient deaths. Efforts to mitigate these issues are of utmost importance.

### CAR T Packaging Overview

### CAR T Sample Collection, Drug Manufacturing and Drug Delivery to Patient

CAR T involves the collection of patient blood (about 30-70 ml). This collection is done at a hospital or draw center. The blood is collected in a bag specifically designed to be frozen at cryogenic temperatures. The bags are made of EVA. Attached are technical data sheets of blood bags used for froze storage. The bags typically have 2-3 ports where tubing is attached.

The blood is collected and then the bag is placed in an aluminum cassette. The cassette is about 1 inch thick and about the size of a DVD case.

The blood bag is subjected to a freezing cycle in the aluminum cassette (gradual controlled rate freezing). The blood is frozen to −120 to −150° C. Once frozen, the cassettes are placed in secondary packaging, with a liquid nitrogen to maintain the blood at −120 to −150° C.

The frozen blood is transported by air, truck to the drug company. At the drug company, the blood is thawed to room temperature and the blood is used to make the CAR T drug. The CAR T drug (30-70 ml) is placed into another blood bag. The CAR T drug in the blood bag is placed into an aluminum cassette. The CAR T drug is frozen. The frozen CAR T drug is placed in in secondary packaging, with a liquid nitrogen to maintain the blood at −120 to −150° C. The CAR T drug is shipped to the hospital. The hospital thaws the CAR T drug and infuses into the patient. The process takes about 25 days to complete.

Problems with CAR T Packaging

The biggest problem with CAR T blood collection is that the EVA bags can chip, crack and break when maintained at cryogenic temperature. At −120 to −150° C., the EVA bags become brittle. One study by a major pharmaceutical company showed 133 failures had been reported to FDA from 2008-2018 of frozen blood bags. The majority of these failures were breakage and were found during the storage of the frozen blood bags. A small handling study with blood bags used for CAR T was conducted related to the effects of fill volume, transport and dropping.
Fill Volume—minimum of 30 ml and maximum of 70 ml.
Transport
Drop—1 meter drop of the frozen blood bag in the cassette
There were three populations of samples—all samples were filled bags, frozen and placed in aluminum cassettes:
(i) Control—no shipping
(ii) Shipped but not dropped
(iii) Shipped and dropped

The control samples did not have any visual failures. Both the shipped and shipped+dropped samples had failures that included: (1) edge chipping of the bag, (2) crack in the bag that extended into the seal (seam) and (3) crack around a sampling port—this failure lead to a leak when the bag was thawed.

To mitigate the impact of sample breakage, they require the patient to provide two bags of blood (they collect a back-up).

The major pharmaceutical company is currently looking at secondary packaging to address the bag breakage issue.

### Opportunities to Improve CAR T Packaging

Any change to the CAR T packaging must be evaluated throughout the process: collection, drug manufacturing, etc.

There is a possibility to use a rigid package (to replace the bags) but a rigid package would have a potentially larger impact on the overall CAR T process.

A unique ID on each bag or container could be beneficial long term as it could potentially eliminate the need for a separate label (and the challenges of adhesion at cryo temperatures).

The coatings of the current disclosure provide a way of changing or optimizing the bag construction without increasing the risk of higher leachables from the bag. The coating would keep the drug contact surface the same while making improvements to the package robustness.

### PECVD Treated Pharmaceutical Packages or Other Vessels

A vessel with a passivation layer or pH protective coating as described herein and/or prepared according to a method described herein can be used for reception and/or storage and/or delivery of a compound or composition. The compound or composition can be sensitive, for example air-sensitive, oxygen-sensitive, sensitive to humidity and/or sensitive to mechanical influences. It can be a biologically active compound or composition, for example a pharmaceutical preparation or medicament like insulin or a composition comprising insulin. A prefilled syringe can be especially considered which contains injectable or other liquid drugs like insulin.

In another aspect, the compound or composition can be a biological fluid, optionally a bodily fluid, for example blood or a blood fraction. In certain aspects of the present disclosure, the compound or composition can be a product to be administrated to a subject in need thereof, for example a product to be injected, like blood (as in transfusion of blood from a donor to a recipient or reintroduction of blood from a patient back to the patient) or insulin.

A vessel with a passivation layer or pH protective coating as described herein and/or prepared according to a method described herein can further be used for protecting a compound or composition contained in its interior space against mechanical and/or chemical effects of the surface of the vessel material. For example, it can be used for preventing or reducing precipitation and/or clotting or platelet activation of the compound or a component of the composition, for example insulin precipitation or blood clotting or platelet activation.

It can further be used for protecting a compound or composition contained in its interior against the environment outside of the pharmaceutical package or other vessel, for example by preventing or reducing the entry of one or more compounds from the environment surrounding the vessel into the interior space of the vessel. Such environmental compound can be a gas or liquid, for example an atmospheric gas or liquid containing oxygen, air, and/or water vapor.

Referring to the Figures, an aspect of the disclosure can be a method in which a barrier coating or layer 30 and a passivation layer or pH protective coating 34 are applied directly or indirectly applied to at least a portion of the interior wall 16 of a vessel such as a bioprocess bag, a bag used for CAR-T cell therapy including CAR-T cell manufacturing or treatment, a process flask, a sample collection tube, for example a blood collection tube and/or a closed-ended sample collection tube; a conduit; a cuvette; or a vessel part, for example a plunger tip, piston, stopper, or seal for contact with and/or storage and/or delivery of a compound or composition.

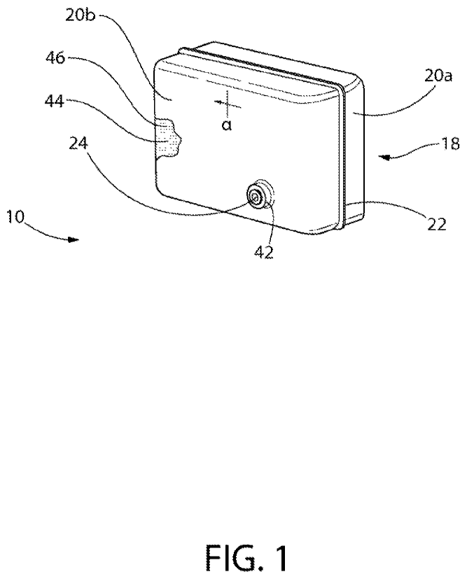

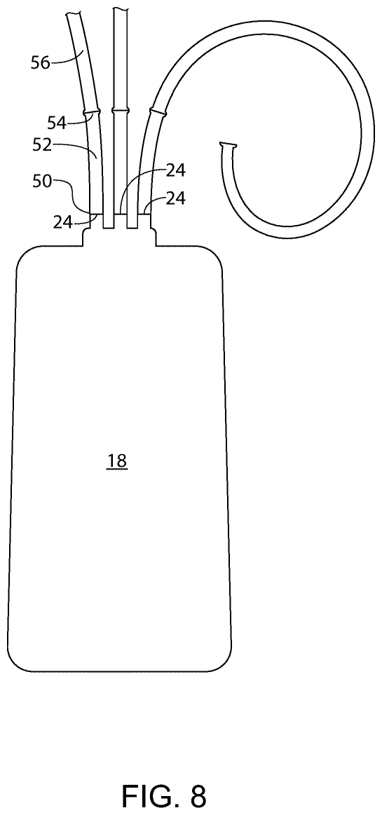

Referring to FIGS. 1 and 8, there is shown an embodiment of a container 10 according to the present disclosure. The container 10 is optionally constructed using standard methods for making wine boxes. Wine boxes generally include wine contained in a plastic bag. The plastic bag is retained in a box (usually cardboard), which provides a protective shell and rigid structure for retaining the bag. Examples of wine boxes and processes for making the same are disclosed in U.S. Pat. Nos. 3,474,933 and 4,274,554 and U.S. Pat. App. Pub. No. 2012/0255971, all of which are incorporated by reference herein in their entireties.

The embodiment of the container 10 according to the present disclosure includes an external package 12 optionally comprising a package body 14 and package lid 16, although a unitary package is also within the scope of the present disclosure. The external package 12 is preferably constructed from an inexpensive rigid or semi-rigid material, such as cardboard, plastic or a soft metal (e.g., aluminum).

The container 10 further includes a sealed flexible bag 18 for containing a liquid, such as a high purity solvent (preferably hexane). The bag, which is retained within the external package 12, is preferably constructed from polyethylene or another thin, flexible polymer with similar physical properties to polyethylene. The flexible bag 18 is made of at least one film sheet 20 having major surface portions 32.

Referring to FIG. 8, a bioprocess bag 18 is shown having three spouts or ports 24 for passing materials in or out of the bag. One or more ports 24 can optionally be made large enough to receive solid reactants or other materials, while one or more ports 24 can be adapted specially for the introduction or removal of liquids. The ports 24 can have fittings 50 to connect tubing such as 52, or tubing such as 52 can be permanently molded in place.

The film sheets 20 may alternatively be packaging laminates of any number of different layers, which can include water vapor sealing layers, support layers, heat sealable layers, decorative layers, print layers, tie layers, and the like. Such laminates are well known in the packaging industry, and need not be described in detail here.

A barrier coating 30 from 2 to 1000 nanometers (nm) thick, optionally from 10 to 200 nm thick, optionally from 20 to 200 nm thick, optionally from 20 to 30 nm thick, is optionally provided on at least one major surface portion 32. For the purposes of the present disclosure the thickness of the SiOx coating or layer or other barrier coating or layer is determined by transmission electron microscopy (TEM). Optionally in any embodiment, the bather coating 30 comprises or consists essentially of SiOx in which x is from about 1.5 to about 2.9, or 1.5 to about 2.6, or about 2, or about 2.3. For the purposes of the present disclosure the value of x, and thus the ratio of silicon to oxygen, is determined by x-ray photoelectron spectroscopy, commonly known as XPS. Optionally, other types of barrier layers can instead be used.

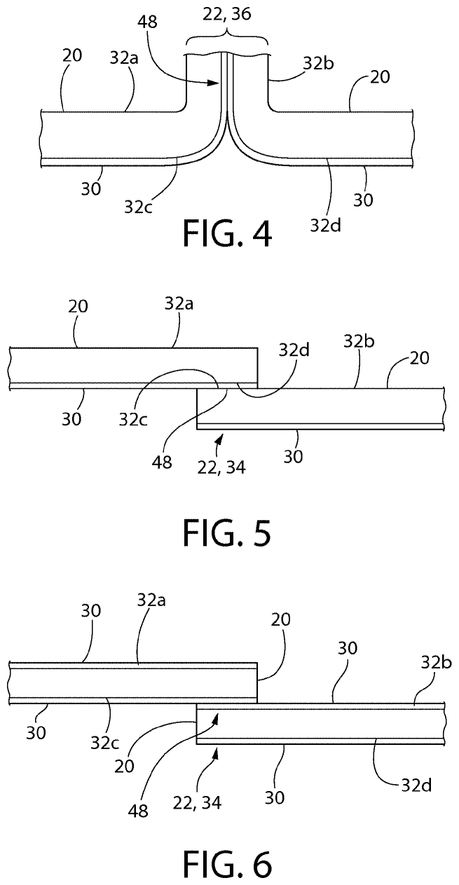

The barrier coating 30 optionally faces the lumen 46, as is desirable when the barrier layer 30 functions to protect the film sheet 20 from the contents of the lumen 46. In an embodiment, the film sheet 20 has first and second major surfaces 32 on opposite sides of the sheet 20 and the barrier coating 30 is on the first major surface 32 only, preferably defining the interior surface, illustrated in FIGS. 4 and 5. Optionally, the barrier coating 30 is coextensive with the first major surface 32, although it could optionally extend into the seal 22 but not all the way to the extreme side edge at the outside of the seal.

Another advantage of providing a barrier coating 30 on the inside surface of the flexible bag 18, is that this protects the barrier coating 30 somewhat from abrasion and other damage during handling and transportation. Optionally in any embodiment, each of the facing major surface portions 32 is at least partially coated with the barrier coating 30. Optionally, each of the facing major surfaces 32 is entirely coated with the barrier coating 30, completely enveloping the lumen 46 without interruption (except in the vicinity of the spout 24, which can be made in such a fashion as to prevent leakage or permeation by the contents of the flexible bag 18). This embodiment is illustrated in FIG. 6, and is also an option in the embodiment of FIG. 4.

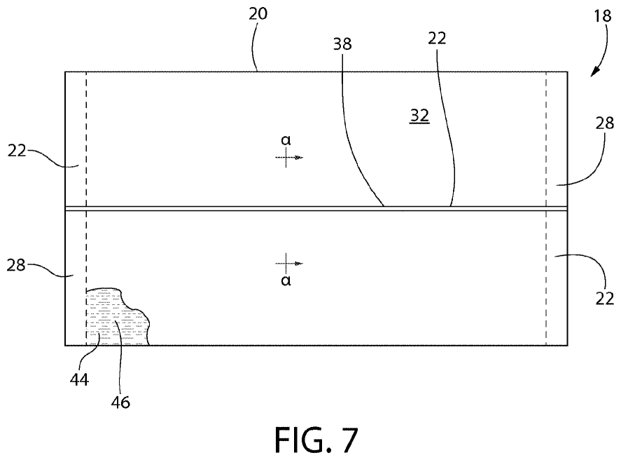

At least one seal 22 is provided between facing major surface portions 32. The reference character 22 in this disclosure or the drawings indicates a seal generically. Seals 22 having various forms are more specifically defined as a face-to-face seal 36 as illustrated in FIG. 4, a lapped seal 34, illustrated in FIGS. 5 and 6, an end seal 28, illustrated in FIG. 7, and a side seal 38, also illustrated in FIG. 7. While end seals 28, side seals 38, and perimeter seals 40 commonly are face-to-face seals, lapped seals 34 can alternatively be used in any embodiment. Other seal types and patterns can also be used, without limitation. At least one film sheet 20 and at least one seal 22 define a flexible bag 18 comprising a lumen 46.

The barrier coating 30 optionally extends into the seal 22. The barrier coating 30 extends into the seal 22 as defined in this specification if, in the seal as assembled, the barrier coating 30 is located between the fused portions 48 of the respective film sheets 20 that are joined. Thus, FIGS. 4, 5, and 6 all illustrate a barrier coating 30 extending into the seal 22. The embodiment of FIG. 4, in which barrier coatings 30 on both sides of the seal 22 extend into the seal is preferred, although the embodiments of FIGS. 5 and 6, in which a barrier coating on only one side of the seal extends into the seal, are also contemplated, particularly when the primary concern is providing a barrier to ingress of oxygen, rather than an internal barrier to egress of the solvent or other fluid contents 44.

It is contemplated that the barrier coating 30, which is extremely thin and has very little volume, will not prevent the use of heat sealing or ultrasonic sealing methods to fuse the adjacent film sheets 20, providing the facing surfaces of the film sheets 20 are directly heat-sealable to each other. It is further contemplated that in the process of heat or ultrasonic sealing, the portion of the barrier coating 30 extending into the seal 22 will be disrupted, allowing direct contact between the adjacent film sheets 20. After sealing, the barrier coating 30 is still regarded as extending into the seal if it was present before the seal was effected, whether or not it can be detected within the finished seal. Alternatively, however, the seal can be effected by placing an adhesive between the surfaces sealed together, as is well known.

The bag 18 is optionally made from a single two dimensional polymer film sheet that is formed into a three dimensional bag. This embodiment is illustrated in FIG. 7, showing a single sheet 20 in which each side has been folded inward, with the free ends of the respective sides registered and sealed together to form the side seal 38. The respective ends have been sealed with end seals 28. Thus, the flexible bag 18 is formed from a single film sheet 20 joined by a side seal 38 and first and second end seals 28.

Alternatively, the bag 18 can be made from two or more separate (originally two dimensional) film sheets 20a, 20b, which are joined together and sealed along a seal (also known as a spine) 22 according to known methods, to form a three dimensional bag 18, as illustrated in FIG. 1. In this embodiment, the two sheets 20a and 20b are joined by a perimeter seal 40.

Optionally, the bag 18 includes an openable spout 24, which is adapted to seat within an opening 26 in the external package 12. A user wishing to release liquid contents (e.g. a high purity solvent) from the bag 18 when the bag 18 holds such contents may open the spout 24.

The bag 18, as described above, is made from one or more two dimensional polymer film sheets. According to an aspect of the present disclosure, before the one or more polymer film sheets are used to construct the bag, they are coated, e.g. on one side or both sides, with an SiOx coating or layer, preferably using plasma enhanced chemical vapor deposition (PECVD). It is contemplated that a two dimensional polymer film sheet, e.g., polyethylene, is an optimal surface on which to apply an SiOx coating or layer because a flat film is less prone to having surface imperfections that can affect the integrity of a SiOx coating than, e.g., the internal surface of a three dimensional container. With fewer such surface imperfections, there is a lower likelihood or incidence of unevenness of coating, missed spots, surface imperfections and cracking, than a conventional three dimensional SiOx coated plastic container. As such, it is contemplated that high purity solvents held in a container according to the present disclosure would have less of an opportunity to contact and attack the polymer substrate of the bag 18 than would a conventional three dimensional SiOx coated plastic container.

Optionally, the SiOx coating may be part of a coating set. For example, a tie coating or layer, a barrier coating or layer, and a pH protective coating or layer, collectively referred to herein as a “trilayer coating,” may be applied to the flexible sheet of the bag. With a trilayer coating, the barrier coating or layer of SiOx optionally is protected against contents having a pH otherwise high enough to remove it by being sandwiched between the pH protective coating or layer and the tie coating or layer, each being optionally an organic layer of SiOxCy as defined in this specification.

Optionally, the tie coating or layer comprises SiOxCy or SiNxCy, preferably can be composed of, comprise, or consist essentially of SiOxCy wherein x is from about 0.5 to about 2.4 and y is from about 0.6 to about 3. The atomic ratios of Si, O, and C in the tie coating or layer 34 optionally can be: Si 100:O 50-150: C 90-200 (i.e. x=0.5 to 1.5, y=0.9 to 2); Si 100:O 70-130: C 90-200 (i.e. x=0.7 to 1.3, y=0.9 to 2); Si 100:O 80-120: C 90-150 (i.e. x=0.8 to 1.2, y=0.9 to 1.5); Si 100:O 90-120: C 90-140 (i.e. x=0.9 to 1.2, y=0.9 to 1.4); or Si 100:O 92-107: C 116-133 (i.e. x=0.92 to 1.07, y=1.16 to 1.33). The atomic ratio can be determined by XPS. Taking into account the H atoms, which are not measured by XPS, the tie coating or layer 34 may thus in one aspect have the formula SiwOxCyHz (or its equivalent SiOxCy), for example where w is 1, x is from about 0.5 to about 2.4, y is from about 0.6 to about 3, and z is from about 2 to about 9. Typically, tie coating or layer 34 would hence contain 36% to 41% carbon normalized to 100% carbon plus oxygen plus silicon.

Optionally, the tie coating or layer can be similar or identical in composition with the pH protective coating or layer described elsewhere in this specification, although this is not a requirement.

Optionally, the tie coating or layer is on average between 5 and 200 nm (nanometers), optionally between 5 and 100 nm, optionally between 5 and 20 nm thick. These thicknesses are not critical. Commonly but not necessarily, the tie coating or layer 34 will be relatively thin, since its function is to change the surface properties of the substrate. Optionally, the tie coating or layer is applied by PECVD, for example of a precursor feed comprising octamethylcyclotetrasiloxane (OMCTS), tetramethyldisiloxane (TMDSO), or hexamethyldisiloxane (HMDSO).

Certain bather coatings or layers such as SiOx as defined here have been found to have the characteristic of being subject to being measurably diminished in barrier improvement factor in less than six months as a result of attack by certain relatively high pH contents of the coated vessel as described elsewhere in this specification, particularly where the barrier coating or layer directly contacts the contents. Barrier layers or coatings of SiOx are eroded or dissolved by some fluids, for example aqueous compositions having a pH above about 5. Since coatings applied by chemical vapor deposition can be very thin tens to hundreds of nanometers thick—even a relatively slow rate of erosion can remove or reduce the effectiveness of the barrier layer in less time than the desired shelf life of a product package. This is particularly a problem for aqueous fluids having a pH of from 4 to 9. The higher the pH of the contents of a coated container or bag, the more quickly it erodes or dissolves the SiOx coating.

The pH protective coating or layer optionally provides protection of the underlying barrier coating or layer against contents of the bag 18 having a pH from 4 to 9, including where a surfactant is present.

Applicant has found that certain pH protective coatings or layers of SiOxCy or SiNxCy formed from polysiloxane precursors, which pH protective coatings or layers have a substantial organic component, do not erode quickly when exposed to fluids, and in fact erode or dissolve more slowly when the fluids have pHs within the range of 4 to 8 or 5 to 9. For example, at pH 8, the dissolution rate of a pH protective coating or layer made from the precursor octamethylcyclotetrasiloxane, or OMCTS, is quite slow. These pH protective coatings or layers of SiOxCy or SiNxCy can therefore be used to cover a barrier layer of SiOx, retaining the benefits of the barrier layer by protecting it from the fluid in the bag. The protective layer is applied over at least a portion of the SiOx layer to protect the SiOx layer from contents stored in a vessel, where the contents otherwise would be in contact with the SiOx layer. The pH protective coating or layer optionally is effective to keep the barrier coating or layer at least substantially undissolved as a result of attack by the fluid for a period of at least six months.

The pH protective coating or layer 38 can be composed of, comprise, or consist essentially of SiwNxCIIz (or its equivalent SiOxCy) or SiwNxCIIz or its equivalent SiNxCy), each as defined previously, preferably SiOxCy, wherein x is from about 0.5 to about 2.4 and y is from about 0.6 to about 3. The atomic ratios of Si, O, and C in the pH protective coating or layer 286 optionally can be: Si 100:O 50-150:C 90-200 (i.e. x=0.5 to 1.5, y=0.9 to 2); Si 100:O70-130:C 90-200 (i.e. x=0.7 to 1.3, y=0.9 to 2); Si 100:O 80-120:C 90-150 (i.e. x=0.8 to 1.2, y=0.9 to 1.5); Si 100:O90-120:C 90-140 (i.e. x=0.9 to 1.2, y=0.9 to 1.4); or Si 100:O 92-107: C 116-133 (i.e. x=0.92 to 1.07, y=1.16 to 1.33); or Si 100:O 80-130:C 90-150.

The thickness of the pH protective coating or layer as applied optionally is between 10 and 1000 nm; alternatively from 10 nm to 900 nm; alternatively from 10 nm to 800 nm; alternatively from 10 nm to 700 nm; alternatively from 10 nm to 600 nm; alternatively from 10 nm to 500 nm; alternatively from 10 nm to 400 nm; alternatively from 10 nm to 300 nm; alternatively from 10 nm to 200 nm; alternatively from 10 nm to 100 nm; alternatively from 10 nm to 50 nm; alternatively from 20 nm to 1000 nm; alternatively from 50 nm to 1000 nm; alternatively from 50 nm to 800 nm; optionally from 50 to 500 nm; optionally from 100 to 200 nm; alternatively from 100 nm to 700 nm; alternatively from 100 nm to 200 nm; alternatively from 300 to 600 nm. The thickness does not need to be uniform throughout the vessel, and will typically vary from the preferred values in portions of a vessel.

Optionally, the pH protective coating or layer is at least coextensive with the barrier coating or layer. The pH protective coating or layer alternatively can be less extensive than the barrier coating, as when the fluid does not contact or seldom is in contact with certain parts of the barrier coating absent the pH protective coating or layer. The pH protective coating or layer alternatively can be more extensive than the barrier coating, as it can cover areas that are not provided with a barrier coating.

The pH protective coating or layer 38 optionally can be applied by plasma enhanced chemical vapor deposition (PECVD) of a precursor feed comprising an acyclic siloxane, a monocyclic siloxane, a polycyclic siloxane, a polysilsesquioxane, a monocyclic silazane, a polycyclic silazane, a polysilsesquiazane, a silatrane, a silquasilatrane, a silproatrane, an azasilatrane, an azasilquasiatrane, an azasilproatrane, or a combination of any two or more of these precursors. Some particular, non-limiting precursors contemplated for such use include octamethylcyclotetrasiloxane (OMCTS).

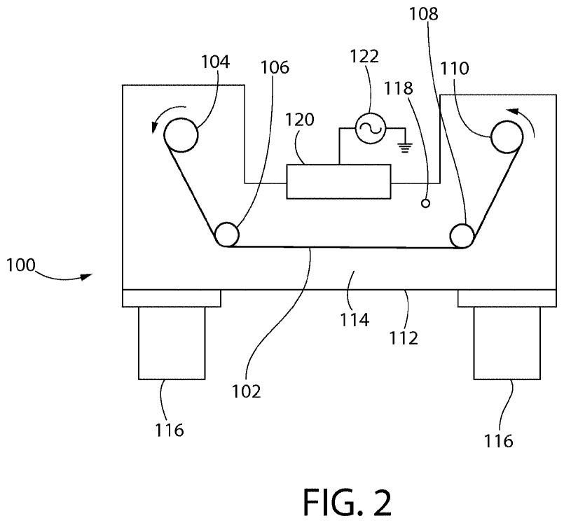

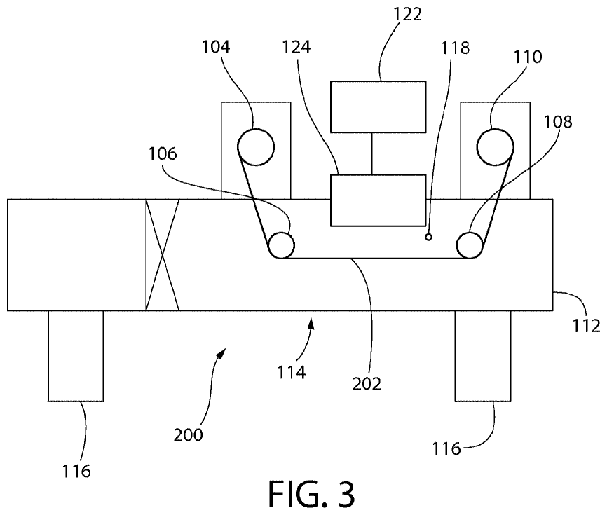

Referring to FIGS. 2 and 3, there are shown alternative embodiments of apparatus 100, 200, which may be used to apply PECVD SiOx coatings to flat flexible polymer films 102, 202, e.g., polyethylene. It is preferred that the coatings are applied in a bulk manufacturing process to rolls of the polymer films. The roll of coated film may then be separated into separate sheets according to known methods for constructing bags.

The respective PECVD coating apparatus of FIGS. 2 and 3 includes an unwind reel 104, guide rollers 106 and 108 and a rewind reel 110 to convey the film 102 or 202 within the chamber 112 through a treatment area 114. The chamber 112 is evacuated to a suitable pressure by the vacuum pumps 116. A gas inlet 118 is provided to introduce chemical vapor deposition precursors and reactants for forming the SiOx or other barrier coating. Plasma is generated in the treatment area 114 of FIG. 2 by an unbalanced magnetron 120 powered by an alternating current power source 122. Plasma is generated in the treatment area 114 of FIG. 3 by a cathode 124.

Processes for applying PECVD coatings to these rolls of film are described, for example in the following articles, which are incorporated herein by reference in their entireties: (1) L. Wood and H. Chatham, “A Comparison of SiO2 Barrier Coated Polypropylene to Other Coated Flexible Substrates,” 35.sup.th Annual Technical Conference Proceedings, Society of Vacuum Coaters (1992); (2) J. Fahlteich, N. Schiller, M. Fahland, S. Straach, S. Gunther, and C. Brantz, “Vacuum Roll-to-Roll Technologies for Transparent Barrier Films,” 54.sup.th Annual Technical Conference Proceedings, Society of Vacuum Coaters, Chicago, Ill. Apr. 16-21, 2011; and (3) J. T. Felts, 36.sup.th Annual Technical Conference Proceedings, Society of Vacuum Coaters (2011).

### Vessel Wall Construction

Optionally, at least a portion of the internal wall 18 of the pharmaceutical package 210 comprises or consists essentially of a polymer, for example a polyolefin (for example a cyclic olefin polymer, a cyclic olefin copolymer, or polypropylene), a polyester, for example polyethylene terephthalate or polyethylene naphthalate, a polycarbonate, polylactic acid, a styrenic polymer or co-polymer or any combination, composite or blend of any two or more of the above materials.

In at least one embodiment, the wall consists of an ethylene vinyl acetate (EVA) and ultra low density polyethylene (ULDPE); or a EVA and linear low density polyethylene (LLDPE), which increases the wall or film's resistance to abrasion, puncture, stretching, and tearing. Optionally, polyethylene vinyl alcohol-copolymers (EVOH) may be utilized separately or together with the above to add a gas barrier. Similarly, a fluid contact material, particularly an ultra low-density polyethylene (ULDPE), may be utilized separately or together with the above. The thickness of these film materials producing the wall may be between 0.00005″ to 0.5″ in thickness, more generally between 0.0005″ to 0.1″ in thickness, between 0.005″ to 0.05″ in thickness, and particularly between 0.01″ to 0.025″ in thickness, each individually or in the aggregate when used together to form the wall of the pharmaceutical package, vessel, or bioprocessing bag or transfer bag or a bag used for CAR-T cell therapy including CAR-T cell manufacturing or treatment.

Other known polymers may be utilized, separately or in combination (including in combination with those described herein), to form the films and walls of the vessel. For example, polyethylene terephthalate (commonly abbreviated PET, PETE, or the obsolete PETP or PET-P PET) and/or polyamide (PA) polymers may be utilized for the present disclosure. In at least one embodiment of the present disclosure, the film materials producing the wall may comprise one or more synthetic polymers. For example, the film materials producing the wall may be a synthetic polymer made of an aliphatic or semi-aromatic polyamide, such as the synthetic polymer commonly referred to Nylon. Nylon is made of repeating units linked by peptide bonds. Commercially, nylon polymer is made by reacting monomers which are either lactams, acid/amines or stoichiometric mixtures of diamines (—NH2) and diacids (—COOH). Mixtures of these can be polymerized together to make copolymers. Nylon polymers can be mixed with a wide variety of additives to achieve many different property variations. Nylon polymers have found significant commercial applications in fabric and fibers (apparel, flooring and rubber reinforcement), in shapes (molded parts for cars, electrical equipment, etc.), and in films (mostly for food packaging). The film materials producing the wall may be one or more such synthetic polymers, or be blends of such materials with other materials.

As an optional feature of any of the foregoing embodiments the polymeric material can be a silicone elastomer or a thermoplastic polyurethane, as two examples, or any material suitable for contact with blood, or with insulin. For example, the use of a coated substrate according to any described embodiment is contemplated for storing insulin.

Optionally, the pharmaceutical package comprises a vessel, such as a bioprocessing bag or a transfer bag or a bag used for CAR-T cell therapy including CAR-T cell manufacturing or treatment, having a wall comprising one or more films. In at least one embodiment, the wall comprises a multi-layer film. The film is put on a roll. The coatings or treatments described herein are then applied using a reel-to-reel PECVD coating process where the coating is applied to at least one side of the film, such as the interior surface of the film or wall.

Optionally, the pharmaceutical package or vessel is a rigid container.

Optionally, the pharmaceutical package comprises a syringe barrel or a cartridge.

Optionally, the pharmaceutical package 210 comprises a vial.

Optionally, the pharmaceutical package 210 comprises a blister package.

Optionally, the pharmaceutical package comprises an ampoule.

Alternatively, the vessel can be a length of tubing from about 1 cm to about 200 cm, optionally from about 1 cm to about 150 cm, optionally from about 1 cm to about 120 cm, optionally from about 1 cm to about 100 cm, optionally from about 1 cm to about 80 cm, optionally from about 1 cm to about 60 cm, optionally from about 1 cm to about 40 cm, optionally from about 1 cm to about 30 cm long, and processing it with a probe electrode as described below. Particularly for the longer lengths in the above ranges, it is contemplated that relative motion between the PECVD or other chemical vapor deposition probe and the vessel can be useful during passivation layer or pH protective coating formation. This can be done, for example, by moving the vessel with respect to the probe or moving the probe with respect to the vessel.

In these embodiments, it is contemplated that the barrier coating or layer discussed below can be thinner or less complete than would be preferred to provide the high gas barrier integrity needed in an evacuated blood collection tube, and thus the long shelf life needed to store a liquid material in contact with the barrier coating or layer for an extended period.

As an optional feature of any of the foregoing embodiments the vessel can have a central axis. As an optional feature of any of the foregoing embodiments the vessel wall can be sufficiently flexible to be flexed at least once at 20° C., without breaking the wall, over a range from at least substantially straight to a bending radius at the central axis of not more than 100 times as great as the outer diameter of the vessel. As an optional feature of any of the foregoing embodiments the bending radius at the central axis can be, for example, not more than 90 times as great as, or not more than 80 times as great as, or not more than 70 times as great as, or not more than 60 times as great as, or not more than 50 times as great as, or not more than 40 times as great as, or not more than 30 times as great as, or not more than 20 times as great as, or not more than 10 times as great as, or not more than 9 times as great as, or not more than 8 times as great as, or not more than 7 times as great as, or not more than 6 times as great as, or not more than 5 times as great as, or not more than 4 times as great as, or not more than 3 times as great as, or not more than 2 times as great as, not more than 1 time as great as, or not more than ½ as great as the outer diameter of the vessel.

As an optional feature of any of the foregoing embodiments the vessel wall can be a fluid-contacting surface made of flexible material.

As an optional feature of any of the foregoing embodiments the vessel lumen can be the fluid flow passage of a pump.

As an optional feature of any of the foregoing embodiments the vessel can be a blood containing vessel. The passivation layer or pH protective coating can be effective to reduce the clotting or platelet activation of blood exposed to the inner or interior surface, compared to the same type of wall uncoated with a hydrophobic layer.

It is contemplated that the incorporation of a hydrophobic layer will reduce the adhesion or clot forming tendency of the blood, as compared to its properties in contact with an unmodified polymeric or SiOx surface. This property is contemplated to reduce or potentially eliminate the need for treating the blood with heparin, as by reducing the necessary blood concentration of heparin in a patient undergoing surgery of a type requiring blood to be removed from the patient and then returned to the patient, as when using a heart-lung machine during cardiac surgery. It is contemplated that this will reduce the complications of surgery involving the passage of blood through such a pharmaceutical package or other vessel, by reducing the bleeding complications resulting from the use of heparin.

Another embodiment can be a vessel including a wall and having an inner or interior surface defining a lumen. The inner or interior surface can have an at least partial passivation layer or pH protective coating that presents a hydrophobic surface, the thickness of the passivation layer or pH protective coating being from monomolecular thickness to about 1000 nm thick on the inner or interior surface, the passivation layer or pH protective coating being effective to reduce the clotting or platelet activation of blood exposed to the inner or interior surface.

Several non-limiting examples of such a vessel are a blood transfusion bag, a blood sample collection vessel in which a sample has been collected, the tubing of a heart-lung machine, a flexible-walled blood collection bag, or tubing used to collect a patient's blood during surgery and reintroduce the blood into the patient's vasculature. If the vessel includes a pump for pumping blood, a particularly suitable pump can be a centrifugal pump or a peristaltic pump. The vessel can have a wall; the wall can have an inner or interior surface defining a lumen. The inner or interior surface of the wall can have an at least partial passivation layer or pH protective coating of a protective layer, which optionally also presents a hydrophobic surface. The passivation layer or pH protective coating can be as thin as monomolecular thickness or as thick as about 1000 nm. Optionally, the vessel can contain blood viable for return to the vascular system of a patient disposed within the lumen in contact with the hydrophobic layer.

An embodiment can be a blood containing vessel including a wall and having an inner or interior surface defining a lumen. The inner or interior surface can have an at least partial passivation layer or pH protective coating that optionally also presents a hydrophobic surface. The passivation layer or pH protective coating can also comprise or consist essentially of SiOxCy where x and y are as defined in this specification. The vessel contains blood viable for return to the vascular system of a patient disposed within the lumen in contact with the hydrophobic coating or layer.

An embodiment can be carried out under conditions effective to form a hydrophobic passivation layer or pH protective coating on the substrate. Optionally, the hydrophobic characteristics of the passivation layer or pH protective coating can be set by setting the ratio of the oxidizing gas to the organosilicon precursor in the gaseous reactant, and/or by setting the electric power used for generating the plasma. Optionally, the passivation layer or pH protective coating can have a lower wetting tension than the uncoated surface, optionally a wetting tension of from 20 to 72 dyne/cm, optionally from 30 to 60 dynes/cm, optionally from 30 to 40 dynes/cm, optionally 34 dyne/cm. Optionally, the passivation layer or pH protective coating can be more hydrophobic than the uncoated surface.

In an optional embodiment, the vessel can have an inner diameter of at least 2 mm, or at least 4 mm.

As an optional feature of any of the foregoing embodiments the vessel can be a tube.

As an optional feature of any of the foregoing embodiments the lumen can have at least two open ends.

Optionally, the pharmaceutical package comprises a vessel, such as a bioprocessing bag or a transfer bag or a bag used for CAR-T cell therapy including CAR-T cell manufacturing or treatment, having a wall comprising one or more films. In at least one embodiment, the wall comprises a multi-layer film. The film is put on a roll. The coatings or treatments described herein are then applied using a reel-to-reel PECVD coating process (aka roll-to-roll process) where the coating is applied to at least one side of the film, such as the interior surface of the film or wall. The fabrication of the film(s) can be achieved using full roll-to-roll (R2R) processes by, for example, either: (i) in a discrete process configuration of one or more machines where each step (e.g., each coating or layer if one or more coatings or layers are applied) can be applied on separate roll-to-roll setups in series or in sequence, or (ii) in an inline process configuration where all the steps (e.g., each coating or layer is applied in one machine all at the same time or in sequence. The main difference is the number of machines (pairs of starting rolls and finished rolls) used to achieve the final finished roll product.

An embodiment of the coating system for the film, wall, or vessel in any embodiment is at least one tie coating or layer, at least one barrier coating or layer, and at least one pH protective coating or layer, and present in any embodiment. This coating or layer set is sometimes known as a “trilayer coating” in which the barrier coating or layer of SiOx is protected against contents having a pH otherwise high enough to remove it by being sandwiched between the pH protective coating or layer and the tie coating or layer, each an organic layer of SiOxCy as defined in this specification.

It will be appreciated that not all three layers of the trilayer coating will necessarily be present, depending on the application and materials used, and that any one or more such coating layers may be included or excluded, or combined with one or more other coatings or layers, while remaining within the embodiments of the present disclosure. The tie coating or layer is optional, as the barrier coating or layer can optionally be directly applied directly to the wall of the bottle 210. The pH protective coating or layer is optional, as it need not be used if the lumen does not contain any liquid contents that tend to erode the barrier coating or layer. For these alternative embodiments, the description of corresponding individual coatings or layers below is applicable.

As another embodiment, the pH protective coating can be applied using PECVD directly on the interior surface of the vessel. As another embodiment, the pH protective coating can be the sole coating on the interior surface of the vessel. The pH protective coating can block extractables/leachables from the wall. The pH protective coating can also provide gas barrier properties. The pH protective coating can also maintain its gas barrier and extractable blocking properties after being stretched.

It is important to characterize the extractables/leachables from the construction polymer materials of the vessels, e.g. single use bioprocess bags. Irgafos 168 is a common antioxidant additive present in many polymers used to form bioprocess bags, which is highly detrimental to cell growth. The extractables resulted from Irgafos 168 can be Irgafos 168 (Mass: 647.46), Irgafos 168 oxide (Mass: 663.46) and Irgafos 168 oxide trimethylamine (TEA) (Mass: 764.57). These components can be characterized by LC-MS spectroscopy.

Specific examples of this trilayer coating in any embodiment are provided in this specification. The contemplated thicknesses of the respective layers in nm (preferred ranges in parentheses) are given in the Trilayer Thickness Table.

Trilayer Thickness Table

| Adhesion | Barrier | Protection |
|---|---|---|
| 5-100 | 20-200 | 50-500 |
| (5-20) | (20-30) | (100-200) |

The trilayer coating set includes as a first layer an adhesion or tie coating or layer that improves adhesion of the barrier coating or layer to the COP substrate. The adhesion or tie coating or layer is also believed to relieve stress on the barrier coating or layer 288, making the barrier layer less subject to damage from thermal expansion or contraction or mechanical shock. The adhesion or tie coating or layer is also believed to decouple defects between the barrier coating or layer and the COP substrate. This is believed to occur because any pinholes or other defects that may be formed when the adhesion or tie coating or layer is applied tend not to be continued when the barrier coating or layer is applied, so the pinholes or other defects in one coating do not line up with defects in the other. The adhesion or tie coating or layer has some efficacy as a barrier layer, so even a defect providing a leakage path extending through the barrier coating or layer is blocked by the adhesion or tie coating or layer.

The trilayer coating set includes as a second layer a barrier coating or layer that provides a barrier to oxygen that has permeated the COP wall. The barrier coating or layer also is a barrier to extraction of the composition of the bottle wall 214 by the contents of the lumen.

The trilayer coating set includes as a third layer a pH protective coating or layer that provides protection of the underlying barrier coating or layer against contents of the syringe, including where a surfactant is present.

The features of each layer of the trilayer coating set are further described below.

### Tie Coating or Layer

The tie coating or layer has at least two functions. One function of the tie coating or layer is to improve adhesion of a barrier coating or layer to a substrate, in particular a thermoplastic substrate. For example, a tie coating or layer, also referred to as an adhesion layer or coating can be applied to the substrate and the barrier layer can be applied to the adhesion layer to improve adhesion of the barrier layer or coating to the substrate.

Another function of the tie coating or layer has been discovered: a tie coating or layer applied under a barrier coating or layer can improve the function of a pH protective coating or layer applied over the barrier coating or layer.

The tie coating or layer can be composed of, comprise, or consist essentially of SiOxCy, in which x is between 0.5 and 2.4 and y is between 0.6 and 3. Alternatively, the atomic ratio can be expressed as the formula SiwOxCy, The atomic ratios of Si, O, and C in the tie coating or layer are, as several options:

Si 100:O 50-150: C 90-200 (i.e. w=1, x=0.5 to 1.5, y=0.9 to 2);
Si 100:O 70-130: C 90-200 (i.e. w=1, x=0.7 to 1.3, y=0.9 to 2)
Si 100:O 80-120: C 90-150 (i.e. w=1, x=0.8 to 1.2, y=0.9 to 1.5)
Si 100:O 90-120: C 90-140 (i.e. w=1, x=0.9 to 1.2, y=0.9 to 1.4), or
Si 100:O 92-107: C 116-133 (i.e. w=1, x=0.92 to 1.07, y=1.16 to 1.33)

The atomic ratio can be determined by XPS. Taking into account the H atoms, which are not measured by XPS, the tie coating or layer may thus in one aspect have the formula SiwOxCyHz (or its equivalent SiOxCy), for example where w is 1, x is from about 0.5 to about 2.4, y is from about 0.6 to about 3, and z is from about 2 to about 9. Typically, tie coating or layer would hence contain 36% to 41% carbon normalized to 100% carbon plus oxygen plus silicon.

Optionally, the tie coating or layer can be similar or identical in composition with the pH protective coating or layer described elsewhere in this specification, although this is not a requirement.

The tie coating or layer is contemplated generally to be from 5 nm to 100 nm thick, preferably from 5 to 20 nm thick, particularly if applied by chemical vapor deposition. These thicknesses are not critical. Commonly but not necessarily, the tie coating or layer will be relatively thin, since its function is to change the surface properties of the substrate.

### Barrier Coating or Layer

In the filled pharmaceutical package or other vessel 210 the barrier coating or layer 30 can be located between the inner or interior surface of the thermoplastic internal wall 16 and the fluid material 40. The barrier coating or layer 286 of SiOx can be supported by the thermoplastic internal wall 16. The barrier coating or layer 286 can have the characteristic of being subject to being measurably diminished in barrier improvement factor in less than six months as a result of attack by the fluid material 40. The barrier coating or layer 286 as described elsewhere in this specification, or in U.S. Pat. No. 7,985,188, or in PCT/US2014/023813 can be used in any embodiment. A silicon-oxide coating is applied using a reel-to-reel PECVD coating process where the coating is applied to at least one side of the film

The barrier coating or layer 30 can be effective to reduce the ingress of atmospheric gas into the lumen 18, compared to an uncoated container otherwise the same as the pharmaceutical package or other vessel 210. The barrier coating or layer for any embodiment defined in this specification (unless otherwise specified in a particular instance) is optionally applied by PECVD as indicated in U.S. Pat. No. 7,985,188 or PCT/US2014/023813.

The barrier improvement factor (BIF) of the barrier coating or layer can be determined by providing two groups of identical containers, adding a barrier coating or layer to one group of containers, testing a barrier property (such as the rate of outgassing in micrograms per minute or another suitable measure) on containers having a barrier coating or layer, doing the same test on containers lacking a barrier coating or layer, and taking a ratio of the properties of the materials a barrier coating or layer versus the materials without a barrier coating or layer. For example, if the rate of outgassing through the barrier coating or layer is one-third the rate of outgassing without a barrier coating or layer, the barrier coating or layer has a BIF of 3.

The barrier coating or layer optionally can be characterized as an “SiOx” coating, and contains silicon, oxygen, and optionally other elements, in which x, the ratio of oxygen to silicon atoms, can be from about 1.5 to about 2.9, or 1.5 to about 2.6, or about 2. These alternative definitions of x apply to any use of the term SiOx in this specification. The barrier coating or layer can be applied, for example to the interior of a pharmaceutical package or other vessel, for example a sample collection tube, a syringe barrel, a vial, or another type of vessel.

The barrier coating or layer 30 comprises or consists essentially of SiOx, from 2 to 1000 nm thick, the barrier coating or layer 30 of SiOx having an interior surface facing the lumen 18 and an outer surface facing the internal wall 16. The barrier coating or layer 30 can be effective to reduce the ingress of atmospheric gas into the lumen 18 compared to an uncoated pharmaceutical package 210. One suitable barrier composition can be one where x is 2.3, for example.

For example, the barrier coating or layer such as 30 of any embodiment can be applied at a thickness of at least 2 nm, or at least 4 nm, or at least 7 nm, or at least 10 nm, or at least 20 nm, or at least 30 nm, or at least 40 nm, or at least 50 nm, or at least 100 nm, or at least 150 nm, or at least 200 nm, or at least 300 nm, or at least 400 nm, or at least 500 nm, or at least 600 nm, or at least 700 nm, or at least 800 nm, or at least 900 nm. The barrier coating or layer can be up to 1000 nm, or at most 900 nm, or at most 800 nm, or at most 700 nm, or at most 600 nm, or at most 500 nm, or at most 400 nm, or at most 300 nm, or at most 200 nm, or at most 100 nm, or at most 90 nm, or at most 80 nm, or at most 70 nm, or at most 60 nm, or at most 50 nm, or at most 40 nm, or at most 30 nm, or at most 20 nm, or at most 10 nm, or at most 5 nm thick. Specific thickness ranges composed of any one of the minimum thicknesses expressed above, plus any equal or greater one of the maximum thicknesses expressed above, are expressly contemplated. The thickness of the SiOx or other barrier coating or layer can be measured, for example, by transmission electron microscopy (TEM), and its composition can be measured by X-ray photoelectron spectroscopy (XPS). The passivation layer or pH protective coating described herein can be applied to a variety of pharmaceutical packages or other vessels made from plastic or glass, for example to plastic tubes, vials, and syringes.

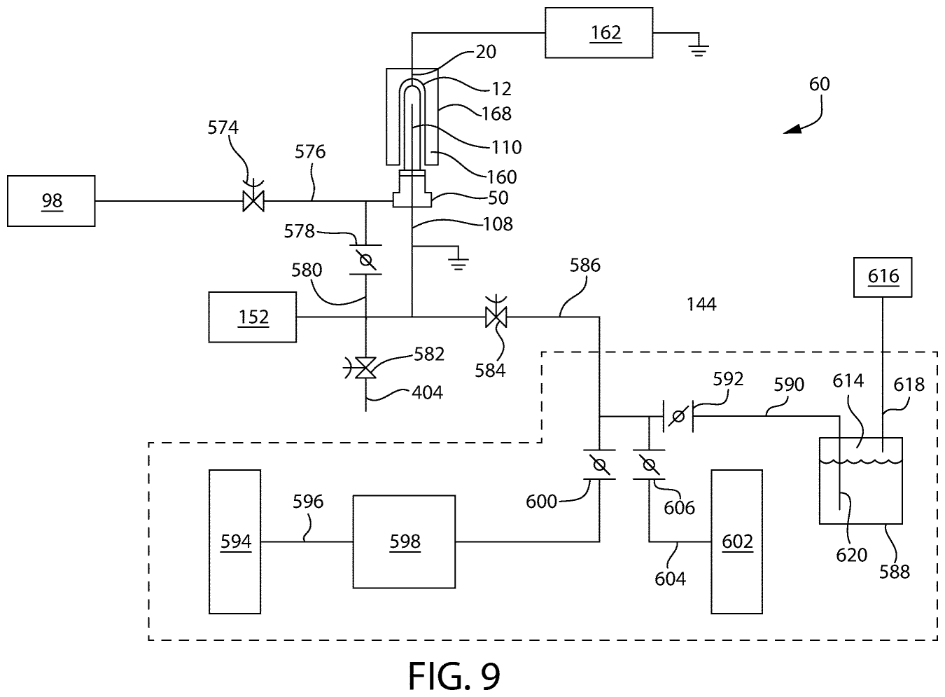

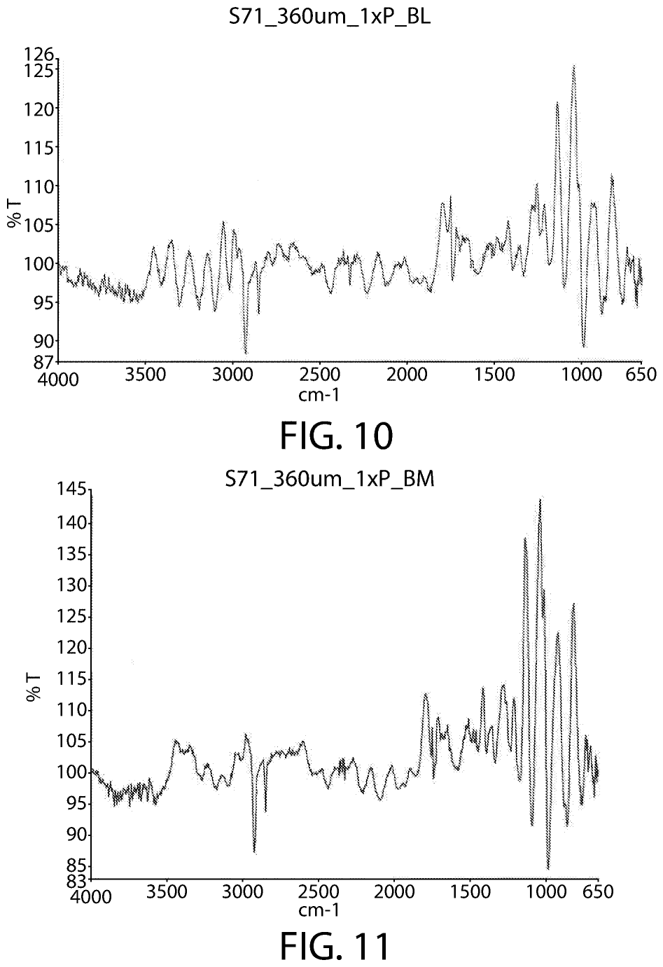

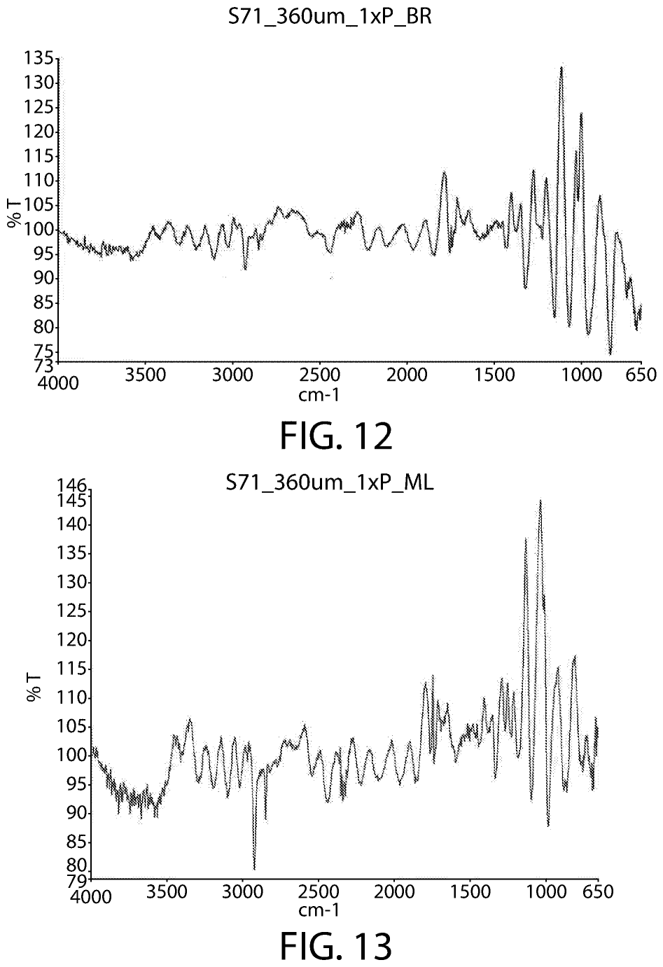

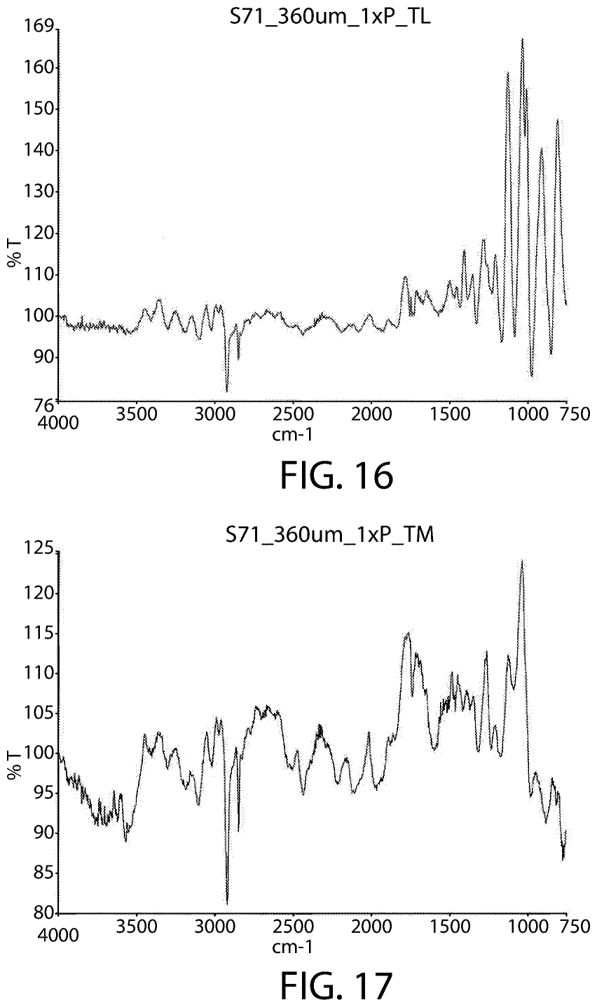

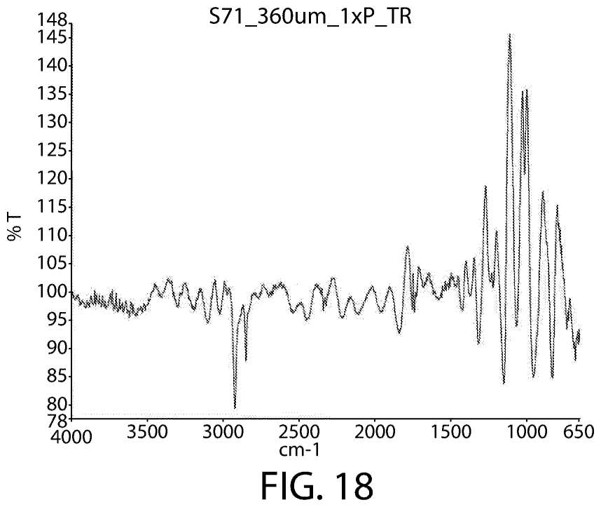

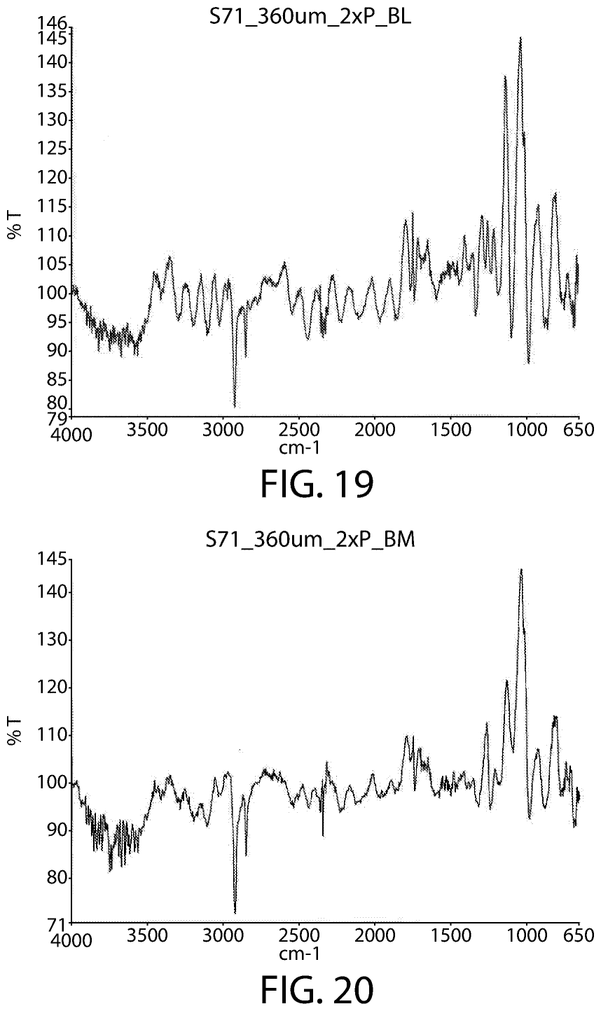

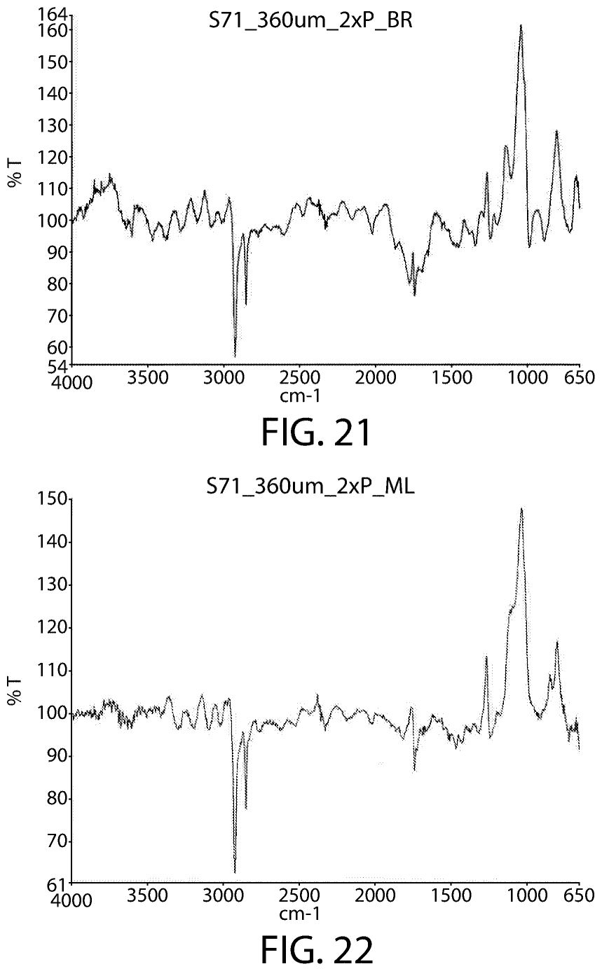

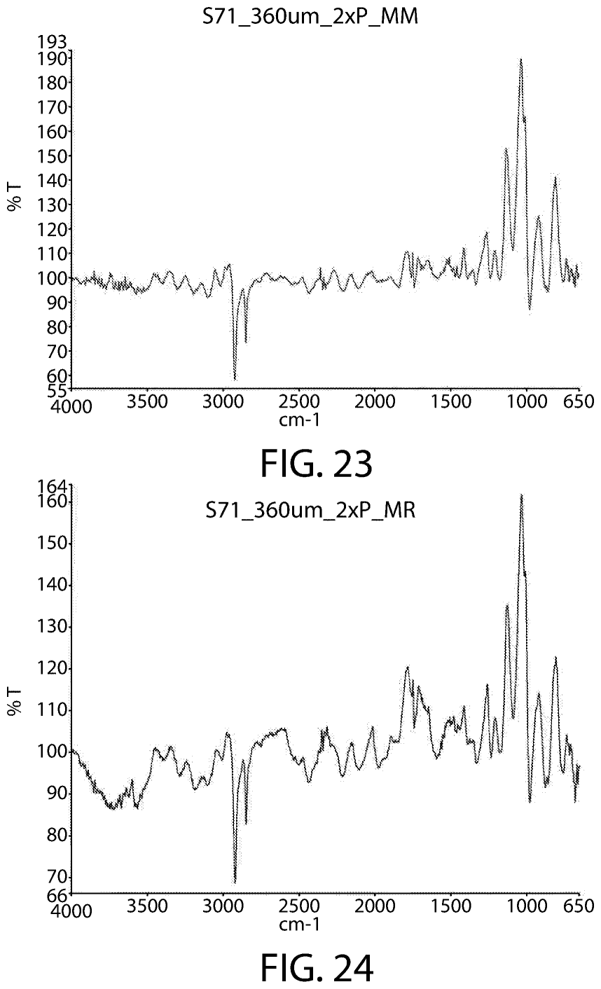

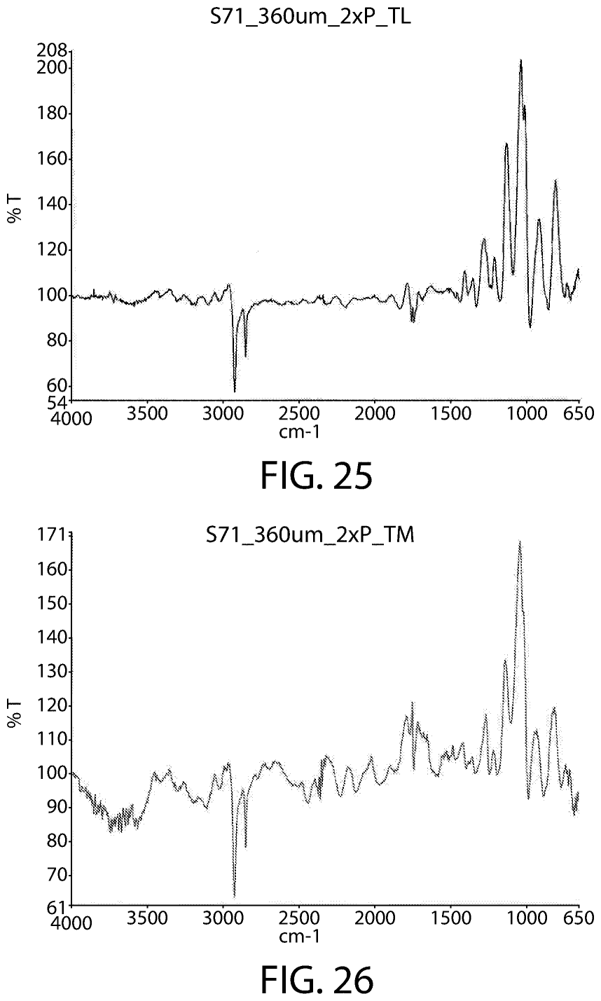

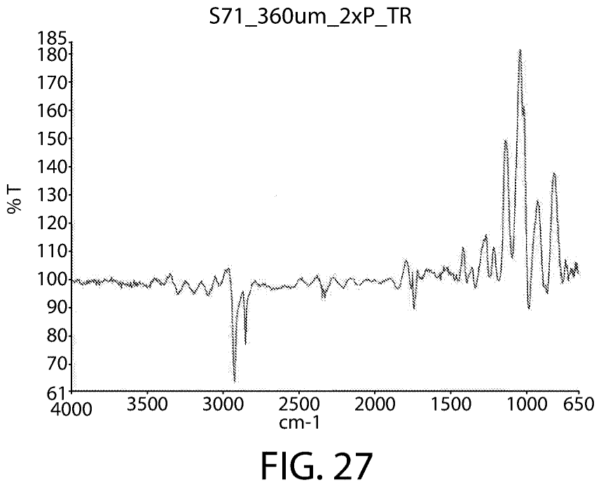

The Fourier Transform Infrared Spectrophotometer (FTIR) absorbance spectrum can provide further information or details regarding the PECVD applied barrier coating. FIGS. 9-18 provide the FTIR absorbance spectrum for 1× (or single) treatments of the barrier coating onto a plastic or polymeric film. FIGS. 19-27 provide the FTIR absorbance spectrum for 2× (or double) treatments of the barrier coating onto a plastic or polymeric film.

### Passivation Layer or pH Protective Coating

A passivation layer or pH protective coating 34 of SiOxCy can be applied, for example, by PECVD directly or indirectly to the barrier coating or layer 30 so it can be located between the barrier coating or layer 30 and the fluid material 40 in the finished article. The passivation layer or pH protective coating 34 can have an interior surface facing the lumen 18 and an outer surface facing the interior surface of the barrier coating or layer 30. The passivation layer or pH protective coating 34 can be supported by the thermoplastic internal wall 16. The passivation layer or pH protective coating 34 can be effective to keep the barrier coating or layer 30 at least substantially undissolved as a result of attack by the fluid material 40 for a period of at least six months, in one non-limiting embodiment.

Optionally, the passivation layer or pH protective coating of SiOxCy can be applied, for example, by PECVD directly on the interior surface of the vessel.

Optionally, the passivation layer or pH protective coating of SiOxCy can be a sole PECVD coating on the interior surface of the vessel.

Optionally, the passivation layer or pH protective coating can be composed of SiwOxCyHz (or its equivalent SiOxCy) or SiwNxCyHz or its equivalent SiNxCy), each as defined in this specification. Taking into account the H atoms, the passivation layer or pH protective coating may thus in one aspect have the formula SiwOxCyHz, or its equivalent SiOxCy, for example where w is 1, x is from about 0.5 to about 2.4, y is from about 0.6 to about 3, and z (if defined) is from about 2 to about 9.

The atomic ratio can be determined by XPS (X-ray photoelectron spectroscopy). XPS does not detect hydrogen atoms, so it is customary, when determining the atomic ratio by XPS, to omit hydrogen from the stated formulation. The formulation thus can be typically expressed as SiwOxCy, where w is 1, x is from about 0.5 to about 2.4, and y is from about 0.6 to about 3, with no limitation on z.

The atomic ratios of Si, O, and C in the “lubricity and/or passivation layer or pH protective coating” can be, as several options:

Si 100:O 50-150: C 90-200 (i.e. w=1, x=0.5 to 1.5, y=0.9 to 2);
Si 100:O 70-130: C 90-200 (i.e. w=1, x=0.7 to 1.3, y=0.9 to 2)
Si 100:O 80-120: C 90-150 (i.e. w=1, x=0.8 to 1.2, y=0.9 to 1.5)
Si 100:O 90-120: C 90-140 (i.e. w=1, x=0.9 to 1.2, y=0.9 to 1.4), or
Si 100:O 92-107: C 116-133 (i.e. w=1, x=0.92 to 1.07, y=1.16 to 1.33)

Typically, such a coating or layer would contain 36% to 41% carbon normalized to 100% carbon plus oxygen plus silicon. Alternatively, the passivation layer or pH protective coating can have atomic concentrations normalized to 100% carbon, oxygen, and silicon, as determined by X-ray photoelectron spectroscopy (XPS) of less than 50% carbon and more than 25% silicon. Alternatively, the atomic concentrations can be from 25 to 45% carbon, 25 to 65% silicon, and 10 to 35% oxygen. Alternatively, the atomic concentrations can be from 30 to 40% carbon, 32 to 52% silicon, and 20 to 27% oxygen. Alternatively, the atomic concentrations can be from 33 to 37% carbon, 37 to 47% silicon, and 22 to 26% oxygen.

Optionally, the atomic concentration of carbon in the protective layer, normalized to 100% of carbon, oxygen, and silicon, as determined by X-ray photoelectron spectroscopy (XPS), can be greater than the atomic concentration of carbon in the atomic formula for the organosilicon precursor. For example, embodiments are contemplated in which the atomic concentration of carbon increases by from 1 to 80 atomic percent, alternatively from 10 to 70 atomic percent, alternatively from 20 to 60 atomic percent, alternatively from 30 to 50 atomic percent, alternatively from 35 to 45 atomic percent, alternatively from 37 to 41 atomic percent.

Optionally, the atomic ratio of carbon to oxygen in the passivation layer or pH protective coating can be increased in comparison to the organosilicon precursor, and/or the atomic ratio of oxygen to silicon can be decreased in comparison to the organosilicon precursor.

Optionally, the passivation layer or pH protective coating can have an atomic concentration of silicon, normalized to 100% of carbon, oxygen, and silicon, as determined by X-ray photoelectron spectroscopy (XPS), less than the atomic concentration of silicon in the atomic formula for the feed gas. For example, embodiments are contemplated in which the atomic concentration of silicon decreases by from 1 to 80 atomic percent, alternatively by from 10 to 70 atomic percent, alternatively by from 20 to 60 atomic percent, alternatively by from 30 to 55 atomic percent, alternatively by from 40 to 50 atomic percent, alternatively by from 42 to 46 atomic percent.

As another option, a passivation layer or pH protective coating is contemplated that can be characterized by a sum formula wherein the atomic ratio C:O can be increased and/or the atomic ratio Si:O can be decreased in comparison to the sum formula of the organosilicon precursor.

The passivation layer or pH protective coating can have a density between 1.25 and 1.65 g/cm3, alternatively between 1.35 and 1.55 g/cm3, alternatively between 1.4 and 1.5 g/cm3, alternatively between 1.4 and 1.5 g/cm3, alternatively between 1.44 and 1.48 g/cm3, as determined by X-ray reflectivity (XRR). Optionally, the organosilicon compound can be octamethylcyclotetrasiloxane and the passivation layer or pH protective coating can have a density which can be higher than the density of a passivation layer or pH protective coating made from HMDSO as the organosilicon compound under the same PECVD reaction conditions.

The passivation layer or pH protective coating optionally can have an RMS surface roughness value (measured by AFM) of from about 2 to about 9, optionally from about 6 to about 8, optionally from about 6.4 to about 7.8. The Ra surface roughness value of the passivation layer or pH protective coating, measured by AFM, can be from about 4 to about 6, optionally from about 4.6 to about 5.8. The Rmax surface roughness value of the passivation layer or pH protective coating, measured by AFM, can be from about 70 to about 160, optionally from about 84 to about 142, optionally from about 90 to about 130.

The rate of erosion, dissolution, or leaching (different names for related concepts) of the construction including a passivation layer or pH protective coating 34, if directly contacted by the fluid material 40, can be less than the rate of erosion, dissolution, or leaching of the barrier coating or layer 30, if directly contacted by the fluid material 40.

The passivation layer or pH protective coating 34 can be effective to isolate or protect the barrier coating or layer 30 from the fluid material 40 at least for sufficient time to allow the barrier coating or layer to act as a barrier during the shelf life of the pharmaceutical package or other vessel 210.

Optionally an FTIR absorbance spectrum of the passivation layer or pH protective coating 34 can have a ratio greater than 0.75 between the maximum amplitude of the Si—O—Si symmetrical stretch peak normally located between about 1000 and 1040 cm-1, and the maximum amplitude of the Si—O—Si asymmetric stretch peak normally located between about 1060 and about 1100 cm-1. Alternatively in any embodiment, this ratio can be at least 0.8, or at least 0.9, or at least 1.0, or at least 1.1, or at least 1.2. Alternatively in any embodiment, this ratio can be at most 1.7, or at most 1.6, or at most 1.5, or at most 1.4, or at most 1.3. Any minimum ratio stated here can be combined with any maximum ratio stated here.

Optionally, the passivation layer or pH protective coating, in the absence of the medicament, can have a non-oily appearance. This appearance has been observed in some instances to distinguish an effective passivation layer or pH protective coating from a lubricity layer, which in some instances has been observed to have an oily (i.e. shiny) appearance.

Optionally, the silicon dissolution rate by a 50 mM potassium phosphate buffer diluted in water for injection, adjusted to pH 8 with concentrated nitric acid, and containing 0.2 wt. % polysorbate-80 surfactant, (measured in the absence of the medicament, to avoid changing the dissolution reagent), at 40° C., can be less than 170 ppb/day. (Polysorbate-80 is a common ingredient of pharmaceutical preparations, available for example as Tween®-80 from Uniqema Americas LLC, Wilmington Del.) As will be seen from the working examples, the silicon dissolution rate can be measured by determining the total silicon leached from the vessel into its contents, and does not distinguish between the silicon derived from the passivation layer or pH protective coating 34, the lubricity layer 287, the barrier coating or layer 30, or other materials present.

Optionally, the silicon dissolution rate can be less than 160 ppb/day, or less than 140 ppb/day, or less than 120 ppb/day, or less than 100 ppb/day, or less than 90 ppb/day, or less than 80 ppb/day. Optionally, in any embodiment of FIGS. 7-9 the silicon dissolution rate can be more than 10 ppb/day, or more than 20 ppb/day, or more than 30 ppb/day, or more than 40 ppb/day, or more than 50 ppb/day, or more than 60 ppb/day. Any minimum rate stated here can be combined with any maximum rate stated here.

Optionally, the total silicon content of the passivation layer or pH protective coating and barrier coating or layer, upon dissolution into a test composition with a pH of 8 from the vessel, can be less than 66 ppm, or less than 60 ppm, or less than 50 ppm, or less than 40 ppm, or less than 30 ppm, or less than 20 ppm.

Optionally, the calculated shelf life of the package (total Si/Si dissolution rate) can be more than six months, or more than 1 year, or more than 18 months, or more than 2 years, or more than 2′/% years, or more than 3 years, or more than 4 years, or more than 5 years, or more than 10 years, or more than 20 years. Optionally, the calculated shelf life of the package (total Si/Si dissolution rate) can be less than 60 years.

Any minimum time stated here can be combined with any maximum time stated here.

The pH protective coating or layer coating or layer described in this specification can be applied in many different ways. For one example, the low-pressure PECVD process described in U.S. Pat. No. 7,985,188 can be used. For another example, instead of using low-pressure PECVD, atmospheric PECVD can be employed to deposit the pH protective coating or layer. For another example, the coating can be simply evaporated and allowed to deposit on the SiOx layer to be protected. For another example, the coating can be sputtered on the SiOx layer to be protected. For still another example, the pH protective coating or layer can be applied from a liquid medium used to rinse or wash the SiOx layer.

### O-Parameter or P-Parameters of Passivation Coating or Protective Layer

The passivation layer or pH protective coating 34 optionally can have an O-Parameter measured with attenuated total reflection (ATR) of less than 0.4, measured as:

$$O \text{-} Parameter = \frac{Intensity at 1253 cm}{Maximum intensity in the range 1000 to 1100 {cm}^{- 1}} .$$

The O-Parameter is defined in U.S. Pat. No. 8,067,070, which claims an O-parameter value of most broadly from 0.4 to 0.9. It can be measured from physical analysis of an FTIR amplitude versus wave number plot to find the numerator and denominator of the above expression, which is the same as FIG. 13 of U.S. Pat. No. 8,067,070, except annotated to show interpolation of the wave number and absorbance scales to arrive at an absorbance at 1253 cm-1 of 0.0424 and a maximum absorbance at 1000 to 1100 cm-1 of 0.08, resulting in a calculated O-parameter of 0.53. The O-Parameter can also be measured from digital wave number versus absorbance data.

U.S. Pat. No. 8,067,070 asserts that its claimed O-parameter range provides a superior passivation layer or pH protective coating, relying on experiments only with HMDSO and HMDSN, which are both non-cyclic siloxanes. Surprisingly, it has been found by the present inventors that if the PECVD precursor is a cyclic siloxane, for example OMCTS, O-parameters outside the ranges claimed in U.S. Pat. No. 8,067,070, using OMCTS, can provide better results than are obtained in U.S. Pat. No. 8,067,070 with HMDSO.

Alternatively, the O-parameter can have a value of from 0.1 to 0.39, or from 0.15 to 0.37, or from 0.17 to 0.35.

Even another aspect of the disclosure can be a composite material as just described, wherein the passivation layer or pH protective coating shows an N-Parameter measured with attenuated total reflection (ATR) of less than 0.7, measured as:

$$N \text{-} Parameter = \frac{Intensity at 840 {cm}^{- 1}}{Intensity at 799 {cm}^{- 1}} .$$

The N-Parameter is also described in U.S. Pat. No. 8,067,070, and can be measured analogously to the O-Parameter except that intensities at two specific wave numbers are used—neither of these wave numbers is a range. U.S. Pat. No. 8,067,070 claims a passivation layer or pH protective coating with an N-Parameter of 0.7 to 1.6. Again, the present inventors have made better coatings employing a passivation layer or pH protective coating 34 having an N-Parameter lower than 0.7, as described above. Alternatively, the N-parameter can have a value of 0.3 to lower than 0.7, or from 0.4 to 0.6, or from at least 0.53 to lower than 0.7.

### Surface Coatings and Layers

Other precursors and methods can be used to apply the pH protective coating or layer or passivating treatment. Similarly, these can be used as a separate surface coatings or layers in addition to or as an alternative to the pH protective coatings or layers described above. To accommodate the latter format, these layers and coatings are referred to herein as surface layers and coatings but may be described herein as a passivation or pH protective treatment. For example, hexamethylene disilazane (HMDZ) can be used as the precursor. HMDZ has the advantage of containing no oxygen in its molecular structure. This passivation treatment is contemplated to be a surface treatment of the SiOx barrier layer with HMDZ. To slow down and/or eliminate the decomposition of the silicon dioxide coatings at silanol bonding sites, the coating must be passivated. It is contemplated that passivation of the surface with HMDZ (and optionally application of a few mono layers of the HMDZ-derived coating) will result in a toughening of the surface against dissolution, resulting in reduced decomposition. It is contemplated that HMDZ will react with the —OH sites that are present in the silicon dioxide coating, resulting in the evolution of NH3 and bonding of S—(CH3)3 to the silicon (it is contemplated that hydrogen atoms will be evolved and bond with nitrogen from the HMDZ to produce NH3).

It is contemplated that this HMDZ passivation can be accomplished through several possible paths.

One contemplated path is dehydration/vaporization of the HMDZ at ambient temperature. First, an SiOx surface is deposited, for example using hexamethylene disiloxane (HNDSO). The as-coated silicon dioxide surface is then reacted with HNDZ vapor. In an embodiment, as soon as the SiOx surface is deposited onto the article of interest, the vacuum is maintained. The HMDSO and oxygen are pumped away and a base vacuum is achieved. Once base vacuum is achieved, HMDZ vapor is flowed over the surface of the silicon dioxide (as coated on the part of interest) at pressures from the mTorr range to many Torr. The HMDZ is then pumped away (with the resulting NH3 that is a by-product of the reaction). The amount of NH3 in the gas stream can be monitored (with a residual gas analyzer—RGA—as an example) and when there is no more NH3 detected, the reaction is complete. The part is then vented to atmosphere (with a clean dry gas or nitrogen). The resulting surface is then found to have been passivated. It is contemplated that this method optionally can be accomplished without forming a plasma.

Alternatively, after formation of the SiOx barrier coating or layer, the vacuum can be broken before dehydration/vaporization of the HMDZ. Dehydration/vaporization of the HMDZ can then be carried out in either the same apparatus used for formation of the SiOx barrier coating or layer or different apparatus.

Dehydration/vaporization of HMDZ at an elevated temperature is also contemplated. The above process can alternatively be carried out at an elevated temperature exceeding room temperature up to about 150° C. The maximum temperature is determined by the material from which the coated part is constructed. An upper temperature should be selected that will not distort or otherwise damage the part being coated.

Dehydration/vaporization of HMDZ with a plasma assist is also contemplated. After carrying out any of the above embodiments of dehydration/vaporization, once the HMDZ vapor is admitted into the part, a plasma is generated. The plasma power can range from a few watts to 100+ watts (similar powers as used to deposit the SiOx). The above is not limited to HMDZ and could be applicable to any molecule that will react with hydrogen, for example any of the nitrogen-containing precursors described in this specification.

Another way of applying the pH protective coating or layer is to apply as the pH protective coating or layer an amorphous carbon or fluorocarbon coating (or a fluorinated hydrocarbon coating), or a combination of the two.

Amorphous carbon coatings can be formed by PECVD using a saturated hydrocarbon, (e.g. methane or propane) or an unsaturated hydrocarbon (e.g. ethylene, acetylene) as a precursor for plasma polymerization. Fluorocarbon coatings (or a fluorinated hydrocarbon coating) can be derived from fluorocarbons (for example, hexafluoroethylene or tetrafluoroethylene). Either type of coating, or a combination of both, can be deposited by vacuum PECVD or atmospheric pressure PECVD.

It is further contemplated that fluorosilicon precursors can be used to provide a pH protective coating or layer over an SiOx barrier layer. This can be carried out by using as a precursor a fluorinated silane precursor such as hexafluorosilane and a PECVD process. The resulting coating would also be expected to be a non-wetting coating.

It is further contemplated that any embodiment of the pH protective coating or layer processes described in this specification can also be carried out without using the article to be coated to contain the plasma.

Yet another coating modality contemplated for protecting or passivating an SiOx barrier layer is coating the barrier layer using a polyamidoamine epichlorohydrin resin. For example, the barrier coated part can be dip coated in a fluid polyamidoamine epichlorohydrin resin melt, solution or dispersion and cured by autoclaving or other heating at a temperature between 60 and 100° C. It is contemplated that a coating of polyamidoamine epichlorohydrin resin can be preferentially used in aqueous environments between pH 5-8, as such resins are known to provide high wet strength in paper in that pH range. Wet strength is the ability to maintain mechanical strength of paper subjected to complete water soaking for extended periods of time, so it is contemplated that a coating of polyamidoamine epichlorohydrin resin on an SiOx barrier layer will have similar resistance to dissolution in aqueous media. It is also contemplated that, because polyamidoamine epichlorohydrin resin imparts a lubricity improvement to paper, it will also provide lubricity in the form of a coating on a thermoplastic surface made of, for example, COC or COP.

Even another approach for protecting an SiOx layer is to apply as a pH protective coating or layer a liquid-applied coating of a polyfluoroalkyl ether, followed by atmospheric plasma curing the pH protective coating or layer. For example, it is contemplated that the process practiced under the trademark TriboGlide®, described in this specification, can be used to provide a pH protective coating or layer that is also a lubricity layer, as TriboGlide® is conventionally used to provide lubricity.

The surface layers and coatings, and the pH protection or passivation coatings and layers, are described herein as protecting an SiOx layer or coating; but that is not required for the embodiments of the present disclosure. The surface layers and coatings, and the pH protection or passivation coatings and layers, may be applied directly to a surface of the wall of the vessel or container or other surface, such as a film or bag.

The preferred drug contact surface includes a coating or layer that provides flexibility while retaining the desirable characteristics of the coatings or layers described herein, including but not limited to moisture barrier, resistance to degradation, compatibility, and the like. Of particular interest is a coating or layer that can provide 1×, 10×, 100×, or larger stretch and elongation of the underlying surface, wall, or film, without detrimentally reducing the desirable characteristics of the coatings or layers described herein, including but not limited to moisture barrier, resistance to degradation, compatibility, and the like. Accordingly, while the embodiments of the present disclosure provide one or more such coatings and layers, other coatings and layers may be contemplated within the scope and breadth of the current disclosure.

In a particular embodiment of the present disclosure, such drug contact surface coating or layer is applied to film materials which comprise one or more synthetic polymers. For example, the film materials producing the wall may be a synthetic polymer made of an aliphatic or semi-aromatic polyamide, such as the synthetic polymer commonly referred to Nylon. Nylon is made of repeating units linked by peptide bonds. Commercially, nylon polymer is made by reacting monomers which are either lactams, acid/amines or stoichiometric mixtures of diamines (—NH₂) and diacids (—COOH). Mixtures of these can be polymerized together to make copolymers. Nylon polymers can be mixed with a wide variety of additives to achieve many different property variations. Nylon polymers have found significant commercial applications in fabric and fibers (apparel, flooring and rubber reinforcement), in shapes (molded parts for cars, electrical equipment, etc.), and in films (mostly for food packaging). The film materials producing the wall may be one or more such synthetic polymers, or be blends of such materials with other materials.

In at least one embodiment, a pharmaceutical package or vessel, for example a bioprocess bag or a transfer bag or a bag used for CAR-T cell therapy including CAR-T cell manufacturing or treatment, comprises:
a polymeric wall having an interior surface and an outer surface;
a tie coating or layer of SiOxCy, wherein x is from about 0.5 to about 2.4 and y is from about 0.6 to about 3, on the interior surface of the wall; and/or
a barrier coating or layer of SiOx, wherein x is from 1.5 to 2.9, on the interior surface of the wall, or when present, the tie coating or layer of SiOxCy; and/or
a passivation layer or pH protective coating of SiOxCy or SiNxCy, wherein x is from about 0.5 to about 2.4 and y is from about 0.6 to about 3, on the interior surface of the wall or, when present, the barrier coating or layer of SiOₓ; and/or
a surface layer or coating of any of, or combination of, the following: silicon-based barrier coating system;
   amorphous carbon coating;
   fluorocarbon coating;
   direct fluorination;
   antiscratch/antistatic coating;
   antistatic coating;
   antistatic additive compound in polymer;
   oxygen scavenging additive compound in polymer;
   colorant additive compound in polymer;
   or antioxidation additive compound in polymer,
   wherein the coating(s) affords improved barrier properties to gases, moisture and solvents and/or the coating(s) is effective to block extractables/leachables from the substrate and any coatings thereon and/or the coating(s) is able to maintain its desirable characteristics described herein against stretching/elongation conditions.

In at least one embodiment, on the interior surface of the pharmaceutical package or vessel, the coating(s) affords improved barrier properties to gases, moisture and solvents and/or the coating(s) is effective to block extractables/leachables from the substrate and any coatings thereon and/or the coating(s) is able to maintain its blocking properties after the coating(s) and the surface thereunder are being stretched/elongated by 5%, optionally 10%, optionally 20%, optionally 30%, optionally 40%, optionally 50%, optionally 70%, optionally 90%, optionally 100%, optionally 150%, optionally 200% of the original size.

In at least one embodiment, on the interior surface of the pharmaceutical package or vessel, the coating(s) affords improved barrier properties to gases, moisture and solvents and maintain the blocking properties after being stretched/elongated.

In at least one embodiment, on the interior surface of the pharmaceutical package or vessel, the coating(s) affords improved barrier properties to gases, moisture and solvents and maintain the blocking properties after being stretched/elongated by 5%, optionally 10%, optionally 20%, optionally 30%, optionally 40%, optionally 50%, optionally 70%, optionally 90%, optionally 100%, optionally 150%, optionally 200% of the original size.

In at least one embodiment, on the interior surface of the pharmaceutical package or vessel, the coating(s) is effective to block extractables/leachables from the substrate and any coatings thereon and maintain the blocking properties after being stretched/elongated.

In at least one embodiment, on the interior surface of the pharmaceutical package or vessel, the coating(s) is effective to block extractables/leachables from the substrate and any coatings thereon and maintain the blocking properties after the coating(s) and the surface under there being stretched/elongated by 5%, optionally 10%, optionally 20%, optionally 30%, optionally 40%, optionally 50%, optionally 70%, optionally 90%, optionally 100%, optionally 150%, optionally 200% of the original size.

In at least one embodiment, the pharmaceutical package or vessel is, for example, a bioprocess bag or a transfer bag or a bag used for CAR-T cell therapy including CAR-T cell manufacturing or treatment, comprising:
a polymeric wall having an interior surface and an outer surface;
a tie coating or layer of SiOxCy, wherein x is from about 0.5 to about 2.4 and y is from about 0.6 to about 3, on the interior surface of the wall;
a barrier coating or layer of SiOx, wherein x is from 1.5 to 2.9, on the tie coating or layer of SiOxCy; and
a passivation layer or pH protective coating of SiOxCy or SiNxCy, wherein x is from about 0.5 to about 2.4 and y is from about 0.6 to about 3, on the barrier coating or layer of SiOₓ;
wherein the coatings are effective to block extractables/leachables from the substrate and any coatings thereon when the coatings and the surface thereunder are not being stretched or after being stretched/elongated.

In at least one embodiment, the pharmaceutical package or vessel is, for example, a bioprocess bag or a transfer bag or a bag used for CAR-T cell therapy including CAR-T cell manufacturing or treatment, comprising:
a polymeric wall having an interior surface and an outer surface;
a tie coating or layer of SiOxCy, wherein x is from about 0.5 to about 2.4 and y is from about 0.6 to about 3, on the interior surface of the wall;
a barrier coating or layer of SiOx, wherein x is from 1.5 to 2.9, on the tie coating or layer of SiOxCy; and
a passivation layer or pH protective coating of SiOxCy or SiNxCy, wherein x is from about 0.5 to about 2.4 and y is from about 0.6 to about 3, on the barrier coating or layer of SiOₓ;
   wherein the coatings are effective to block extractables/leachables from the substrate and any coatings thereon after the coatings and the surface thereunder being stretched/elongated by 5%, optionally 10%, optionally 25%, optionally 30%, optionally 40%, optionally 50%, optionally 70%, optionally 90%, optionally 100%, optionally 150%, optionally 200% of the original size.

In at least one embodiment, the pharmaceutical package or vessel is, for example, a bioprocess bag or a transfer bag or a bag used for CAR-T cell therapy including CAR-T cell manufacturing or treatment, comprising:
a polymeric wall having an interior surface and an outer surface; and
a passivation layer or pH protective coating of SiOxCy or SiNxCy, wherein x is from about 0.5 to about 2.4 and y is from about 0.6 to about 3, on the interior surface of the wall;
   wherein the coating is effective to block extractables/leachables from the substrate after the coating and the surface thereunder being stretched/elongated by 5%, optionally 10%, optionally 20%, optionally 25%, optionally 30%, optionally 40%, optionally 50%, optionally 70%, optionally 90%, optionally 100%, optionally 150%, optionally 200% of the original size.

Optionally, the vessels are flexible and stretchable.

Optionally it is desired to limit the stretching of at least a portion of the vessel, for example a bag, in order to obtain the optimum properties, such as sealing, blocking or barrier properties. It is contemplated that any supportive structures can be used for this purpose, for example, any rigid supportive structure, such as a frame, a rigid box, a wine box type structure.

### PECVD Apparatus

The low-pressure PECVD process described in U.S. Pat. No. 7,985,188 can be used to provide the barrier coating or layer, lubricity coating or layer, and/or passivation layer or pH protective coating described in this specification. A brief synopsis of that process follows, with reference to present FIG. 1.

A PECVD apparatus or coating station 60 suitable for the present purpose includes a vessel holder 50, an inner electrode defined by the probe 108, an outer electrode 160, and a power supply 162. The pre-assembly 12 seated on the vessel holder 50 defines a plasma reaction chamber, which optionally can be a vacuum chamber. Optionally, a source of vacuum 98, a reactant gas source 144, a gas feed (probe 108) or a combination of two or more of these can be supplied.

The PECVD apparatus can be used for atmospheric-pressure PECVD, in which case the plasma reaction chamber defined by the pre-assembly 12 does not need to function as a vacuum chamber.

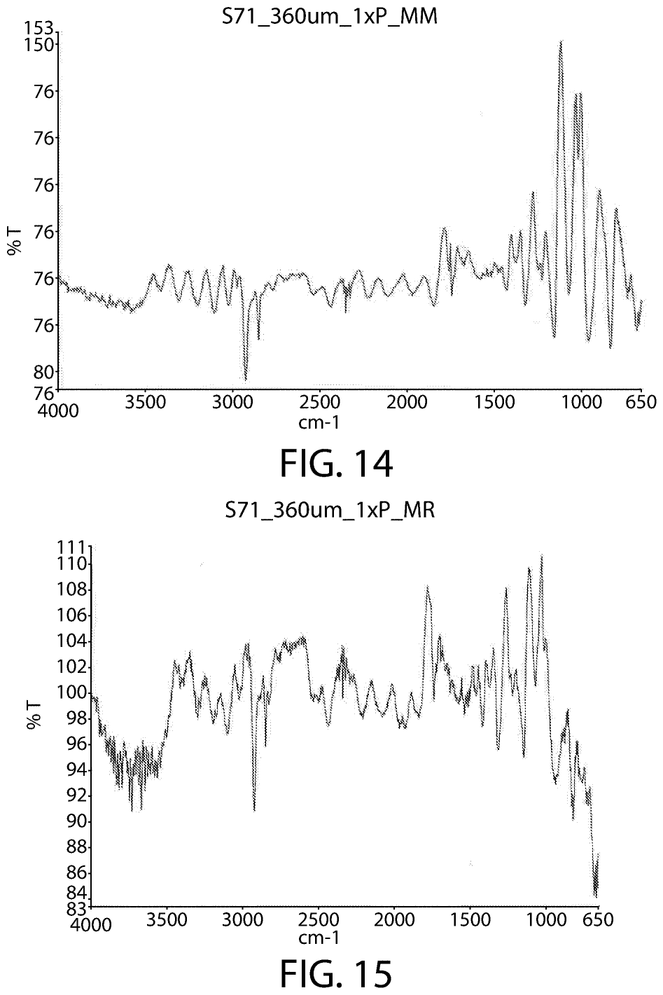

Referring to FIG. 14, the vessel holder 50 comprises a gas inlet port 104 for conveying a gas into the pre-assembly 12 seated on the opening 82. The gas inlet port 104 can have a sliding seal provided for example by at least one O-ring 106, or two O-rings in series, or three O-rings in series, which can seat against a cylindrical probe 108 when the probe 108 is inserted through the gas inlet port 104. The probe 108 can be a gas inlet conduit that extends to a gas delivery port at its distal end 110. The distal end 110 of the illustrated embodiment can be inserted at an appropriate depth in the pre-assembly 12 for providing one or more PECVD reactants and other precursor feed or process gases.

FIG. 9 shows additional optional details of the coating station 60 that are usable, for example, with all the illustrated embodiments. The coating station 60 can also have a main vacuum valve 574 in its vacuum line 576 leading to the pressure sensor 152. A manual bypass valve 578 can be provided in the bypass line 580. A vent valve 582 controls flow at the vent 404.

Flow out of the PECVD gas or precursor source 144 can be controlled by a main reactant gas valve 584 regulating flow through the main reactant feed line 586. One component of the gas source 144 can be the organosilicon liquid reservoir 588, containing the precursor. The contents of the reservoir 588 can be drawn through the organosilicon capillary line 590, which optionally can be provided at a suitable length to provide the desired flow rate. Flow of organosilicon vapor can be controlled by the organosilicon shut-off valve 592. Pressure can be applied to the headspace 614 of the liquid reservoir 588, for example a pressure in the range of 0-15 psi (0 to 78 cm. Hg), from a pressure source 616 such as pressurized air connected to the headspace 614 by a pressure line 618 to establish repeatable organosilicon liquid delivery that is not dependent on atmospheric pressure (and the fluctuations therein). The reservoir 588 can be sealed and the capillary connection 620 can be at the bottom of the reservoir 588 to ensure that only neat organosilicon liquid (not the pressurized gas from the headspace 614) flows through the capillary tube 590. The organosilicon liquid optionally can be heated above ambient temperature, if necessary or desirable to cause the organosilicon liquid to evaporate, forming an organosilicon vapor. To accomplish this heating, the apparatus can advantageously include heated delivery lines from the exit of the precursor reservoir to as close as possible to the gas inlet into the syringe. Preheating can be useful, for example, when feeding OMCTS.

Oxidant gas can be provided from the oxidant gas tank 594 via an oxidant gas feed line 596 controlled by a mass flow controller 598 and provided with an oxidant shut-off valve 600.

Optionally in any embodiment, other precursor, oxidant, and/or carrier gas reservoirs such as 602 can be provided to supply additional materials if needed for a particular deposition process. Each such reservoir such as 602 can have an appropriate feed line 604 and shut-off valve 606.

The processing station 60 can include an electrode 160 fed by a radio frequency power supply 162 for providing an electric field for generating plasma within the pre-assembly 12 during processing. In this embodiment, the probe 108 can be electrically conductive and can be grounded, thus providing a counter-electrode within the pre-assembly 12. Alternatively, in any embodiment the outer electrode 160 can be grounded and the probe 108 can be directly connected to the power supply 162.

The outer electrode 160 can either be generally cylindrical or a generally U-shaped elongated channel. Each embodiment can have one or more sidewalls, such as 164 and 166, and optionally a top end 168, disposed about the pre-assembly 12 in close proximity.

### Equipment

### PECVD Apparatus for Forming PECVD Coating or Layer

PECVD apparatus, a system and precursor materials suitable for applying any of the PECVD coatings or layers described in this specification, specifically including the tie coating or layer 289, the barrier coating or layer 288, or the pH protective coating or layer 286 is described in described in U.S. Pat. No. 7,985,188, which is incorporated by reference.

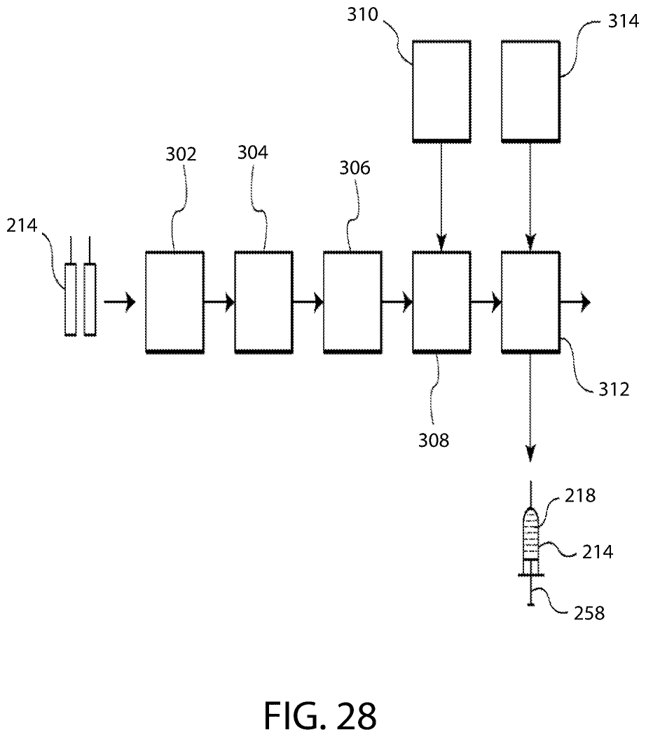

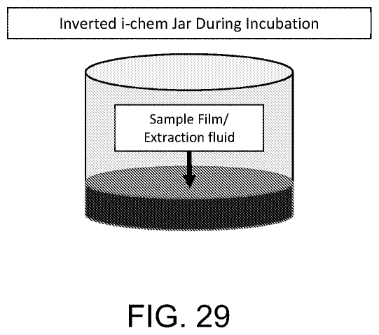

An overview of these conditions is provided in FIG. 28, which shows a vessel processing system adapted for making such a vessel. The vessels having walls 214 can be conveyed to a tie coater 302, which is suitable apparatus for applying a tie coating or layer to the interior surface of the wall, such as the PECVD apparatus described in U.S. Pat. No. 7,985,188.

Optionally, the vessels can then be conveyed to a barrier coater 304, which is suitable apparatus for applying a barrier coating or layer to the interior surface of the wall, such as the PECVD apparatus described in U.S. Pat. No. 7,985,188 or PCT/US2014/023813.

The vessels can then be conveyed to a pH protective coater 306, which is suitable apparatus for applying a pH protective coating or layer to the interior surface of the wall, such as the PECVD apparatus described in U.S. Pat. No. 7,985,188 or PCT/US2014/023813. This then completes the coating set.

Optionally, further steps can be carried out by the system. For example, the coated vessels can be conveyed to a fluid filler 308 which places fluid from a fluid supply 310 into the lumens of the coated vessels.

For another example the filled vessels can be conveyed to a closure installer 312, which takes closures, for example plungers or stoppers, from a closure supply 314 and seats them in the lumens of the coated vessels.

In any embodiment of the disclosure, the tie coating or layer optionally can be applied by plasma enhanced chemical vapor deposition (PECVD).

In any embodiment of the disclosure, the barrier coating or layer optionally can be applied by PECVD.

In any embodiment of the disclosure, the pH protective coating or layer optionally can be applied by PECVD.

In any embodiment of the disclosure, the vessel can comprise or consist of a syringe barrel, a vial, cartridge or a blister package.

Reaction conditions for forming the SiOx barrier layer are described in U.S. Pat. No. 7,985,188, which is incorporated by reference.

The tie or adhesion coating or layer can be produced, for example, using as the precursor tetramethyldisiloxane (TMDSO) or hexamethyldisiloxane (HMDSO) at a flow rate of 0.5 to 10 sccm, preferably 1 to 5 sccm; oxygen flow of 0.25 to 5 sccm, preferably 0.5 to 2.5 sccm; and argon flow of 1 to 120 sccm, preferably in the upper part of this range for a 1 mL syringe and the lower part of this range for a 5 ml. vial. The overall pressure in the vessel during PECVD can be from 0.01 to 10 Torr, preferably from 0.1 to 1.5 Torr. The power level applied can be from 5 to 100 Watts, preferably in the upper part of this range for a 1 mL syringe and the lower part of this range for a 5 ml. vial. The deposition time (i.e. “on” time for RF power) is from 0.1 to 10 seconds, preferably 1 to 3 seconds. The power cycle optionally can be ramped or steadily increased from 0 Watts to full power over a short time period, such as 2 seconds, when the power is turned on, which may improve the plasma uniformity. The ramp up of power over a period of time is optional, however.

The pH protective coating or layer 286 coating or layer described in this specification can be applied in many different ways. For one example, the low-pressure PECVD process described in U.S. Pat. No. 7,985,188 can be used. For another example, instead of using low-pressure PECVD, atmospheric PECVD can be employed to deposit the pH protective coating or layer. For another example, the coating can be simply evaporated and allowed to deposit on the SiOx layer to be protected. For another example, the coating can be sputtered on the SiOx layer to be protected. For still another example, the pH protective coating or layer 286 can be applied from a liquid medium used to rinse or wash the SiOx layer.

Other precursors and methods can be used to apply the pH protective coating or layer or passivating treatment. For example, hexamethylene disilazane (HNDZ) can be used as the precursor. HMDZ has the advantage of containing no oxygen in its molecular structure. This passivation treatment is contemplated to be a surface treatment of the SiOx barrier layer with HMDZ. To slow down and/or eliminate the decomposition of the silicon dioxide coatings at silanol bonding sites, the coating must be passivated. It is contemplated that passivation of the surface with HMDZ (and optionally application of a few mono layers of the HMDZ-derived coating) will result in a toughening of the surface against dissolution, resulting in reduced decomposition. It is contemplated that HMDZ will react with the —OH sites that are present in the silicon dioxide coating, resulting in the evolution of NH3 and bonding of S—(CH3)3 to the silicon (it is contemplated that hydrogen atoms will be evolved and bond with nitrogen from the HMDZ to produce NH3).

It is contemplated that this HMDZ passivation can be accomplished through several possible paths.

One contemplated path is dehydration/vaporization of the HMDZ at ambient temperature. First, an SiOx surface is deposited, for example using hexamethylene disiloxane (HNDSO). The as-coated silicon dioxide surface is then reacted with HMDZ vapor. In an embodiment, as soon as the SiOx surface is deposited onto the article of interest, the vacuum is maintained. The HMDSO and oxygen are pumped away and a base vacuum is achieved. Once base vacuum is achieved, HMDZ vapor is flowed over the surface of the silicon dioxide (as coated on the part of interest) at pressures from the mTorr range to many Torr. The HMDZ is then pumped away (with the resulting NH3 that is a byproduct of the reaction). The amount of NH3 in the gas stream can be monitored (with a residual gas analyzer—RGA—as an example) and when there is no more NH3 detected, the reaction is complete. The part is then vented to atmosphere (with a clean dry gas or nitrogen). The resulting surface is then found to have been passivated. It is contemplated that this method optionally can be accomplished without forming a plasma.

Alternatively, after formation of the SiOx barrier coating or layer, the vacuum can be broken before dehydration/vaporization of the HMDZ. Dehydration/vaporization of the HMDZ can then be carried out in either the same apparatus used for formation of the SiOx barrier coating or layer or different apparatus.

Dehydration/vaporization of HMDZ at an elevated temperature is also contemplated. The above process can alternatively be carried out at an elevated temperature exceeding room temperature up to about 150° C. The maximum temperature is determined by the material from which the coated part is constructed. An upper temperature should be selected that will not distort or otherwise damage the part being coated.

Dehydration/vaporization of HMDZ with a plasma assist is also contemplated. After carrying out any of the above embodiments of dehydration/vaporization, once the HMDZ vapor is admitted into the part, a plasma is generated. The plasma power can range from a few watts to 100+ watts (similar powers as used to deposit the SiOx). The above is not limited to HMDZ and could be applicable to any molecule that will react with hydrogen, for example any of the nitrogen-containing precursors described in this specification.

Another way of applying the pH protective coating or layer is to apply as the pH protective coating or layer an amorphous carbon or fluorocarbon coating, or a combination of the two.

Amorphous carbon coatings can be formed by PECVD using a saturated hydrocarbon, (e.g. methane or propane) or an unsaturated hydrocarbon (e.g. ethylene, acetylene) as a precursor for plasma polymerization. Fluorocarbon coatings can be derived from fluorocarbons (for example, hexafluoroethylene or tetrafluoroethylene). Either type of coating, or a combination of both, can be deposited by vacuum PECVD or atmospheric pressure PECVD. It is contemplated that that an amorphous carbon and/or fluorocarbon coating will provide better passivation of an SiOx barrier layer than a siloxane coating since an amorphous carbon and/or fluorocarbon coating will not contain silanol bonds.

It is further contemplated that fluorosilicon precursors can be used to provide a pH protective coating or layer over an SiOx barrier layer. This can be carried out by using as a precursor a fluorinated silane precursor such as hexafluorosilane and a PECVD process. The resulting coating would also be expected to be a non-wetting coating.

It is further contemplated that any embodiment of the pH protective coating or layer processes described in this specification can also be carried out without using the article to be coated to contain the plasma. For example, external surfaces of medical articles, for example catheters, surgical instruments, closures, and others can be protected or passivated by sputtering the coating, employing a radio frequency target.

Yet another coating modality contemplated for protecting or passivating an SiOx barrier layer is coating the barrier layer using a polyamidoamine epichlorohydrin resin. For example, the barrier coated part can be dip coated in a fluid polyamidoamine epichlorohydrin resin melt, solution or dispersion and cured by autoclaving or other heating at a temperature between 60 and 100° C. It is contemplated that a coating of polyamidoamine epichlorohydrin resin can be preferentially used in aqueous environments between pH 5-8, as such resins are known to provide high wet strength in paper in that pH range. Wet strength is the ability to maintain mechanical strength of paper subjected to complete water soaking for extended periods of time, so it is contemplated that a coating of polyamidoamine epichlorohydrin resinon an SiOx barrier layer will have similar resistance to dissolution in aqueous media. It is also contemplated that, because polyamidoamine epichlorohydrin resin imparts a lubricity improvement to paper, it will also provide lubricity in the form of a coating on a thermoplastic surface made of, for example, COC or COP.

Even another approach for protecting an SiOx layer is to apply as a pH protective coating or layer a liquid-applied coating of a polyfluoroalkyl ether, followed by atmospheric plasma curing the pH protective coating or layer. For example, it is contemplated that the process practiced under the trademark TriboGlide®, described in this specification, can be used to provide a pH protective coating or layer that is also a lubricity layer, as TriboGlide® is conventionally used to provide lubricity.

Exemplary PECVD reaction conditions for preparing a pH protective coating or layer 286 in a 3 ml sample size syringe with a ⅛″ diameter tube (open at the end) are as follows:

For depositing a pH protective coating or layer, a precursor feed or process gas can be employed having a standard volume ratio of, for example:
from 0.5 to 10 standard volumes, optionally from 1 to 6 standard volumes, optionally from 2 to 4 standard volumes, optionally equal to or less than 6 standard volumes, optionally equal to or less than 2.5 standard volumes, optionally equal to or less than 1.5 standard volumes, optionally equal to or less than 1.25 standard volumes of the precursor, for example OMCTS or one of the other precursors of any embodiment;
from 0 to 100 standard volumes, optionally from 1 to 200 standard volumes, optionally from 1 to 80 standard volumes, optionally from 5 to 100 standard volumes, optionally from 10 to 70 standard volumes, of a carrier gas of any embodiment, for example argon.
from 0.1 to 10 standard volumes, optionally from 0.1 to 2 standard volumes, optionally from 0.2 to 1.5 standard volumes, optionally from 0.2 to 1 standard volumes, optionally from 0.5 to 1.5 standard volumes, optionally from 0.8 to 1.2 standard volumes of an oxidizing agent.
   The power level can be, for example, from 0.1-500 watts.
   Specific Flow rates and power levels contemplated include:

| | |
|---|---|
| OMCTS: | 2.0 sccm |
| Oxygen: | 0.7 sccm |
| Argon: | 7.0 sccm |
| Power: | 3.5 watts |

### Application of Barrier Coating or Layer

When carrying out the present method, a barrier coating or layer 30 can be applied directly or indirectly to at least a portion of the internal wall 16 of the barrel 14. In the illustrated embodiment, the barrier coating or layer 30 can be applied while the pre-assembly 12 is capped, though this is not a requirement. The barrier coating or layer 30 can be an SiOx barrier coating or layer applied by plasma enhanced chemical vapor deposition (PECVD), under conditions substantially as described in U.S. Pat. No. 7,985,188. The barrier coating or layer 30 can be applied under conditions effective to maintain communication between the barrel lumen 18 and the dispensing portion lumen 26 via the proximal opening 22 at the end of the applying step.

In any embodiment the barrier coating or layer 30 optionally can be applied through the opening 32.

In any embodiment the barrier coating or layer 30 optionally can be applied by introducing a vapor-phase precursor material through the opening and employing chemical vapor deposition to deposit a reaction product of the precursor material on the internal wall of the barrel.

In any embodiment the precursor material for forming the barrier coating optionally can be any of the precursors described in U.S. Pat. No. 7,985,188 or in this specification for formation of the passivating layer or pH protective coating.

In any embodiment the reactant vapor material optionally can comprise an oxidant gas.

In any embodiment the reactant vapor material optionally can comprise oxygen.

In any embodiment the reactant vapor material optionally can comprise a carrier gas.

In any embodiment the reactant vapor material optionally can include helium, argon, krypton, xenon, neon, or a combination of two or more of these.

In any embodiment the reactant vapor material optionally can include argon.

In any embodiment the reactant vapor material optionally can be a precursor material mixture with one or more oxidant gases and a carrier gas in a partial vacuum through the opening and employing chemical vapor deposition to deposit a reaction product of the precursor material mixture on the internal wall of the barrel.

In any embodiment the reactant vapor material optionally can be passed through the opening at sub-atmospheric pressure.

In any embodiment plasma optionally can be generated in the barrel lumen 18 by placing an inner electrode into the barrel lumen 18 through the opening 32, placing an outer electrode outside the barrel 14 and using the electrodes to apply plasma-inducing electromagnetic energy which optionally can be radio frequency energy, in the barrel lumen 18. If a different arrangement is used, the plasma-inducing electromagnetic energy can be microwave energy or other forms of electromagnetic energy.

In any embodiment the electromagnetic energy optionally can be direct current. In any embodiment the electromagnetic energy optionally can be alternating current. The alternating current optionally can be modulated at frequencies including audio, or microwave, or radio, or a combination of two or more of audio, microwave, or radio.

In any embodiment the electromagnetic energy optionally can be applied across the barrel lumen (18).

The recipe for the PECVD coating is as follows:

| Test ID | | Delay (s) | | | | |
|---|---|---|---|---|---|---|
| (2017- | Delay (s) | (With | O2 | HMDSO | RF | Duration |
| 109-M) | (No Gas) | Gas) | (SCCM) | (SCCM) | (W) | (s) |
| 1 | 15 | 15 | 10 | 10 | 300 | 60 |
| 2 | 15 | 15 | 10 | 10 | 300 | 120 |

### Application of Passivation Layer or pH Protective Coating

In any embodiment, in addition to applying a first coating or layer as described above, the method optionally can include applying second or further coating or layer of the same material or a different material. As one example useful in any embodiment, particularly contemplated if the first coating or layer is an SiOx barrier coating or layer, a further coating or layer can be placed directly or indirectly over the barrier coating or layer. One example of such a further coating or layer useful in any embodiment is a passivation layer or pH protective coating 34.

Optionally, the passivation layer or pH protective layer can be applied directly on the interior surface of the vessel. Optionally, the pH protective coating is the sole coating on the interior surface of the vessel.

### Application of Surface Layers or Coatings

In any embodiment, in addition or as a substitute to applying one or more of the coatings or layers as described above, the method optionally can include applying a surface layer or coating of the same material or a different material. As one example useful in any embodiment, particularly contemplated if the first coating or layer is an SiOx barrier coating or layer, a further coating or layer can be placed directly or indirectly over the barrier coating or layer. One example of such a further coating or layer useful in any embodiment is a surface layer or coating of a fluorinated hydrocarbon (fluorocarbon coating). Alternatively, a surface layer or coating may be applied directly to the wall or surface of the vessel, container, film, or bag.

The PECVD coating apparatus and process are as described generally in PECVD protocols of U.S. Pat. No. 7,985,188, PCT/US16/47622, or PCT/US2014/023813. The entire text and drawings of U.S. Pat. No. 7,985,188, PCT/US16/47622 and PCT/US2014/023813 are incorporated here by reference.

In one embodiment of the disclosure, the tie or adhesion coating or layer and the barrier coating or layer, and optionally the pH protective layer, are applied in the same apparatus, without breaking vacuum between the application of the adhesion coating or layer and the barrier coating or layer or, optionally, between the barrier coating or layer and the pH protective coating or layer. During the process, a partial vacuum is drawn in the lumen. While maintaining the partial vacuum unbroken in the lumen, a tie coating or layer of SiOxCy is applied by a tie PECVD coating process. The tie PECVD coating process is carried out by applying sufficient power to generate plasma within the lumen while feeding a gas suitable for forming the coating. The gas feed includes a linear siloxane precursor, optionally oxygen, and optionally an inert gas diluent. The values of x and y are as determined by X-ray photoelectron spectroscopy (XPS). Then, while maintaining the partial vacuum unbroken in the lumen, the plasma is extinguished. A tie coating or layer of SiOxCy, for which x is from about 0.5 to about 2.4 and y is from about 0.6 to about 3, is produced on the inside surface as a result.

Later during the process, while maintaining the partial vacuum unbroken in the lumen, a barrier coating or layer is applied by a barrier PECVD coating process. The barrier PECVD coating process is carried out by applying sufficient power to generate plasma within the lumen while feeding a gas. The gas feed includes a linear siloxane precursor and oxygen. A barrier coating or layer of SiOx, wherein x is from 1.5 to 2.9 as determined by XPS is produced between the tie coating or layer and the lumen as a result.

Then optionally, while maintaining the partial vacuum unbroken in the lumen, the plasma is extinguished.

Later, as a further option, a pH protective coating or layer of SiOxCy can be applied. In this formula as well, x is from about 0.5 to about 2.4 and y is from about 0.6 to about 3, each as determined by XPS. The pH protective coating or layer is optionally applied between the barrier coating or layer and the lumen, by a pH protective PECVD coating process. This process includes applying sufficient power to generate plasma within the lumen while feeding a gas including a linear siloxane precursor, optionally oxygen, and optionally an inert gas diluent.

Optionally in any embodiment, the PECVD process for applying the tie coating or layer, the barrier coating or layer, and/or the pH protective coating or layer, or any combination of two or more of these, is carried out by applying pulsed power (alternatively the same concept is referred to in this specification as “energy”) to generate plasma within the lumen.

Alternatively, the tie PECVD coating process, or the barrier PECVD coating process, or the pH protective PECVD coating process, or any combination of two or more of these, can be carried out by applying continuous power to generate plasma within the lumen.

### Trilayer Coating Process Protocol (all Layers Coated in the Same Apparatus):

The trilayer coating as described in this embodiment of the disclosure is applied by adjusting the flows of a single organosilicon monomer (HMDSO) and oxygen and also varying the PECVD generating power between each layer (without breaking vacuum between any two layers).

The vessel (here a 6 mL COP vial) is placed on a vessel holder, sealed, and a vacuum is pulled within the vessel. Vials are used to facilitate storage while containing fluid as indicated below. Proportional results are contemplated if blood sample collection tubes are used. After pulling vacuum, the gas feed of precursor, oxygen, and argon is introduced, then at the end of the “plasma delay” continuous (i.e. not pulsed) RF power at 13.56 MHz is turned on to form the tie coating or layer. Then power is turned off, gas flows are adjusted, and after the plasma delay power is turned on for the second layer—an SiOx barrier coating or layer. This is then repeated for a third layer before the gases are cut off, the vacuum seal is broken, and the vessel is removed from the vessel holder. The layers are put down in the order of Tie then Barrier then pH Protective. An exemplary process settings are as shown in the following table.

| | O₂ | Ar | HMDSO | Power | Deposition |
|---|---|---|---|---|---|
| Coating | (sccm) | (sccm) | (sccm) | (W) | Time (sec) |
| Tie | 1 | 40 | 2 | 20 | 2.5 |
| Barrier | 100 | 0 | 1 | 60 | 15 |
| PH | 1 | 40 | 2 | 20 | 10 |
| Protective | | | | | |

As a still further alternative, pulsed power can be used for some steps, and continuous power can be used for others. For example, when preparing a trilayer coating or layer composed of a tie coating or layer, a barrier coating or layer, and a pH protective coating or layer, an option specifically contemplated for the tie PECVD coating process and for the pH protective PECVD coating process is pulsed power, and an option contemplated for the corresponding barrier layer is using continuous power to generate plasma within the lumen.

### Forming and Welding of the Pharmaceutical Package

Either before or after the film of the pharmaceutical package, particularly a polymeric film, has been coated or treated by the coatings described above, the film must be formed into the desired pharmaceutical package configuration. When the pharmaceutical package is a bioprocessing bag or a transfer bag, such as a bag used for CAR-T cell therapy including CAR-T cell manufacturing or treatment an aseptic transfer bag, as described herein, the film that forms the wall of the bag may be coated before or after it is shaped into a bag configuration by the coatings processes described above. A film may be shaped into its final pharmaceutical package or vessel configuration by a number of known means, including heat staking, fusing, sewing, hot molding, cold molding, injection molding, extrusion, welding, ultrasonic welding, or laser welding (including, as described herein).

In at least one embodiment of the present disclosure, a laser welding system that allows clear-to-clear plastic welding without the need for laser absorbing additives is utilized. This system incorporates a micron-scale laser, such as a 2 micron laser, with a greatly increased absorption by clear polymers and enables a highly controlled melting through the thickness of optically clear parts. The system utilizes, in at least one embodiment, a programmable multi-axes servo gantry and a scan head to control the action of both components moving the beam. This assures highly precise and controllable beam delivery when welding mid-size and large components. This system is designed to provide clear-to-clear laser welding solutions to produce the pharmaceutical packages, vessel, and other surfaces described in the embodiments of the present application, including bioprocessing bags, transfer bags, or a bag used for CAR-T cell therapy including CAR-T cell manufacturing or treatment.

Optionally, the pharmaceutical package comprises a vessel, such as a bioprocessing bag or a transfer bag or a bag used for CAR-T cell therapy including CAR-T cell manufacturing or treatment, having a wall comprising one or more films. In at least one embodiment, the wall comprises a multi-layer film. The film is put on a roll. The coatings or treatments described herein are then applied using a reel-to-reel PECVD coating process (aka roll-to-roll process) where the coating is applied to at least one side of the film, such as the interior surface of the film or wall. The fabrication of the film(s) can be achieved using full roll-to-roll (R2R) processes by, for example, either: (i) in a discrete process configuration of one or more machines where each step (e.g., each coating or layer if one or more coatings or layers are applied) can be applied on separate roll-to-roll setups in series or in sequence, or (ii) in an inline process configuration where all the steps (e.g., each coating or layer is applied in one machine all at the same time or in sequence. The main difference is the number of machines (pairs of starting rolls and finished rolls) used to achieve the final finished roll product.

Optionally the pharmaceutical package comprises an coated Ethylene-vinyl acetate (EVA) bag.

Ethylene-vinyl acetate (EVA), is the copolymer of ethylene and vinyl acetate. EVA materials are “rubber-like” in softness and flexibility. The material has good clarity and gloss, low-temperature toughness, stress-crack resistance, hot-melt adhesive waterproof properties, and resistance to UV radiation. EVA materials find many applications in medical devices, for example Macopharma's EVA Bags. These EVA bags can be used for CAR-T cell therapy.

Cell therapies, termed “living drugs” for their capacity to dynamically and temporally respond to changes during their production ex vivo and after their administration in vivo, are very promising in recent cancer treatment. Genetically engineered chimeric antigen receptor (CAR) T cells have rapidly developed into powerful tools to harness the power of immune system manipulation against cancer. Regulatory agencies are beginning to approve CAR T cell therapies due to their striking efficacy in treating some hematological malignancies (Biotechnol J. 2018 February; 13(2); doi:10.1002/biot.201700095).

The typical CAR T cell manufacturing process begins with harvesting the patient's peripheral blood mononuclear cells (PBMCs) through leukapheresis. The cells are cryopreserved in blood bags and shipped frozen, then thawed and activated after arrival at the manufacturing facility.

During CAR T cell manufacturing process, a bioreactor including a bioprocessing bag (e.g. Cellbag®, Flexsafe®) is often used. In the current disclosure, these bioprocessing bags can be coated.

Once the film is formed, and optionally coated with one or more coatings or layers, the film may be formed into an intermediate or final configuration—such as a bag. One or more of the methods described herein may be used to form the desired configuration, such as by heat staking, fusing, sewing, hot molding, cold molding, injection molding, extrusion, welding, ultrasonic welding, or laser welding (including, as described herein). The desired configuration may be formed before or after the coating stages or steps are performed. If the forming is to occur after the coating stages or steps, i.e., once a coating or layer of SiOx, SiOxCy, and/or SiNxCy is applied, the final shape may be achieved by a number of methods. In at least one embodiment, the coated film may be cuffed (i.e., bent over itself) such that plastic substrate surfaces (instead of the coated surfaces) are able to contact each other and then joined such as by heat staking, fusing, sewing, hot molding, cold molding, injection molding, extrusion, welding, ultrasonic welding, or laser welding. Alternatively, a method such as high speed laser welding (e.g., femtosecond laser welding) could be used to join either the plastic substrate surfaces or the coated surfaces.

Additionally or alternatively, the film could be masked, either passively or actively, during the coating process to enable suitable surfaces to be joined to form the desired configuration. For example, active masking such as with a tape, removable or irremovable coating or layer, or other material that prevents a coating or layer of SiOx, SiOxCy, and/or SiNxCy from being applied to the substrate may be used to enable suitable surfaces to be joined to form the desired configuration. Additionally or alternatively, passive masking such as computer-assisted coaters or detectors may be utilized to ensure certain areas of the film are not coated. For example, the coatings systems may use computers to preserve certain portions, such as edge portions for example, of the film from receiving one or more coatings. The computers may be preprogrammed to identify the uncoated locations of the film. Additionally or alternatively, detectors such as mechanical or optical detectors may be utilized to preserve or identify uncoated portions of the substrate surface. Once the films are processed and the uncoated portions are identified, the plastic substrate surfaces (instead of the coated surfaces) are able to contact each other and then joined such as by heat staking, fusing, sewing, hot molding, cold molding, injection molding, extrusion, welding, ultrasonic welding, or laser welding. The entire film manufacturing, coating, masking, joining, and final forming of the desired configuration may be achieved in one or more machines, such as the roll-to-roll processes described herein.

### Handling of the Pharmaceutical Package

Single use bioreactor packages are used for manufacturing biopharmaceutical drugs. The packages are intended for single use. They range in size from 50 L to 10,000 L. The more common sizes are from 500 L-5,000 L, used in the manufacturing of biopharmaceuticals.

Most single use bioreactor packages comprise components made of polymeric materials, which together create a system or unit operation designed for one-time or campaign use. Single-use bioreactor bags are self-contained, preassembled and usually gamma irradiated for sterility and ready-to-use. Single-use assemblies can be customized to meet defined applications and unit operations.

The packages are designed to stretch up to 200% without breaking. This is intended to address any stretching of the bag during shipping, filling and processing.

The bioreactor packages are made of a multi-layer polymer. These polymers have additives (e.g. anti-oxidants) that can leach into the drug. There is a need to eliminate/block leachables. The silicon-based barrier coating system of the current disclosure eliminates/reduces the leachables from the polymer packages. In order to optimize the blocking function of the coated packages, another embodiment of the disclosure is a method of handling the silicon-based coated, single use bioreactor packages comprising limiting the stretching of the packages during manufacturing, packaging, filling, processing and transporting of the packages.

The silicon-based coating comprises:
a tie coating or layer of SiOxCy, wherein x is from about 0.5 to about 2.4 and y is from about 0.6 to about 3, on the interior surface of the wall; and/or
a barrier coating or layer of SiOₓ, wherein x is from 1.5 to 2.9, on the interior surface of the wall, or when present, the tie coating or layer of SiOxCy; and/or
a passivation layer or pH protective coating of SiOxCy or SiNxCy, wherein x is from about 0.5 to about 2.4 and y is from about 0.6 to about 3, on the interior surface of the wall or, when present, the barrier coating or layer of SiOₓ; and/or
a surface layer or coating of any of, or combination of, the following: silicon-based barrier coating system;
   amorphous carbon coating;
   fluorocarbon coating;
   direct fluorination;
   antiscratch/antistatic coating;
   antistatic coating;
   antistatic additive compound in polymer;
   oxygen scavenging additive compound in polymer;
   colorant additive compound in polymer;
   or antioxidation additive compound in polymer.

One aspect of the disclosure is a method to limit the stretching of the silicon-based coating coated packages which comprises avoiding folding or avoiding sharp creases, optionally putting the package or vessel in
a tube or sleeve; or
a rigid frame, optionally made of stainless steel; or
a flexible intermediate bulk container (FIBC), optionally made of a flexible fabric, optionally with four loops on each of the top four corners; or
on a pallet, optionally lifted up from underneath

FIBCs are large containers made of a flexible fabric, usually with four loops on each of the top four corners. When filled with material, these containers can weigh up to 2000 pounds or more. The loops are designed to be placed around the forks of a forklift to move the container from one location to another. The package can also be placed on a pallet and lift it up from underneath, which places considerably less stress on the package itself.

When the container is empty, it weighs nothing more than five to seven pounds. However, the container combined with its contents can weigh as much as 2000 pounds, and so the container can transport a full metric ton of material. Although reusable, due to the low cost of these containers, users tend to cut the containers open when they are ready to pour the transported material out. These containers are economical, inexpensive to own, and make managing loose, flowing materials simple and easy.

The single use, coated bioreactor packages would be fitted inside the FIBC. These packages come in many different structural forms with various electrostatic properties. Structural forms refer to how the package is made, its different features, and the specifications that comprise its structure.

### Pressure Monitoring and Controlling

Another aspect of the disclosure is to incorporate a pressure monitoring system and/or release valve in the single use bioreactor package to prevent an increased pressure in the bag that can cause the package to burst.

One key difference between single use bioreactor packages and traditional stainless-steel piping systems is the pressure tolerance of the plastic components, which is generally lower than that of their stainless-steel counterparts. Challenges of high-pressure operations with single-use assemblies have been reported in several applications: e.g., a sterile filtration integrity test, a final fill system, and a high-viscosity concentrated product flow stream. A system's pressure rating is defined as its maximum allowable internal working pressure, whether for a vessel, tank, or piping used to hold or transport liquids or gases. It depends on the component's materials of construction.

The following reasons advocate for development of a single-use assembly pressure design guideline to accommodate bioprocessing conditions. First, tubing pressure performance data are reported only in terms of burst pressure. Second, expansion under pressure of tubing's internal diameter (ID) has not been considered for adapter connections, although an increase in internal volume of tubing is not permitted in some applications. Third, although fasteners for connecting tubes and adapters/connectors are expected to maintain system integrity and prevent leaks, no comprehensive pressure performance data for assemblies have yet been resolved.

One aspect of the disclosure is that the package comprises a pressure device. Optionally, the pressure can be monitored by a pressure gauge installed in one of the ports of the single use bioreactor package. For single use bioreactors, one example of a pressure sensor is PendoTECH, Princeton, N.J. This sensor can monitor the pressure in the range of 1 psi and less. Optionally, the pressure devices are compatible with gamma irradiation up to 50 KGy so they can be placed on the bioreactor before it is gamma sterilized.

The package or vessel of the current disclosure can be used in the entire process of Cart-T drug preparation and treatment. The package or vessel maintains its integrity and its desired properties during the entire process. The contents in the package or vessel also maintain their integrity and activities.

One example is described as the following.

Optionally as described in “Facts About Chimeric Antigen Receptor (CAR)T-Cell Therapy” published by Leukemia & Lymphoma Society, revised June 2018, Car-T cell therapy involves the following steps:

1. Patients are evaluated to determine if CAR T-cell therapy is safe and appropriate.
2. T cells are harvested from the patient by leukapheresis, optionally contained in a bag or a rigid container of the current disclosure. Depending on the product or clinical trial, the bag or container may be frozen and shipped to a Good Manufacturing Practice (GMP) facility for further processing.
3. The T cells are activated by being placed in culture and are exposed to antibody-coated beads in order to activate them.
4. The CAR gene is introduced into activated T cells in vitro. Viral vectors can be used.
5. The CAR T cells are expanded in vitro. Finally, the CAR T cells, are optionally introduced into a bag or a rigid container of the current disclosure and are optionally frozen for shipment to the infusion site.
6. The patient undergoes “preconditioning” chemotherapy.
7. The CAR T cells, optionally contained in the bag or rigid container of the current disclosure, are thawed and infused back into the patient.

In another embodiment a pressure relief valve (check valve) is installed in the single use bioreactor bag to ensure that the pressure does not exceed a maximum threshold.

### EXAMPLES

### Example 1

The purpose of this example was to compare the extractable level of a pH protective layer coated film versus that of an uncoated film.

10×10 cm² Square LLDPE film samples were coated according to the pH protective coating method described in the specification. After the completion of the coating process, the coated samples were taken straight from the coater and set-up with limited handling. For each of the coated film sample, a circle was “punched” out of the square film to fit snugly inside the PTFE lined lid of an i-chem glass sample jar (43.2 mm opening). The jar was filled with 3.0 ml of extraction fluid (EtOH). The i-chem lid was then secured onto the mouth of jar with the coated side of the film exposed to the inside of the jar. The surface area of the film in contact with the extraction fluid (EtOH) is 14.66 cm² (surface area/volume ratio of 4.9 cm²/ml). The jars were then placed in the incubation oven (50° C.) inverted so that the extraction fluid (EtOH) was in contact with the film. After the completion of the extraction, the extraction solution was analyzed using LC-MS spectroscopy. EtOH blanks were prepared in chromatography vials and incubated with the samples side by side.

The above extraction procedures were repeated with uncoated LLDPE film samples using the extraction fluid (EtOH) and incubated in the same way as described above.

After 18 hrs of incubation, the extraction fluid from each jar was then transferred to a 2 ml chromatography vial and analyzed via LC-MS. EtOH blanks were ran after every sample to verify the LC-MS system was clean.

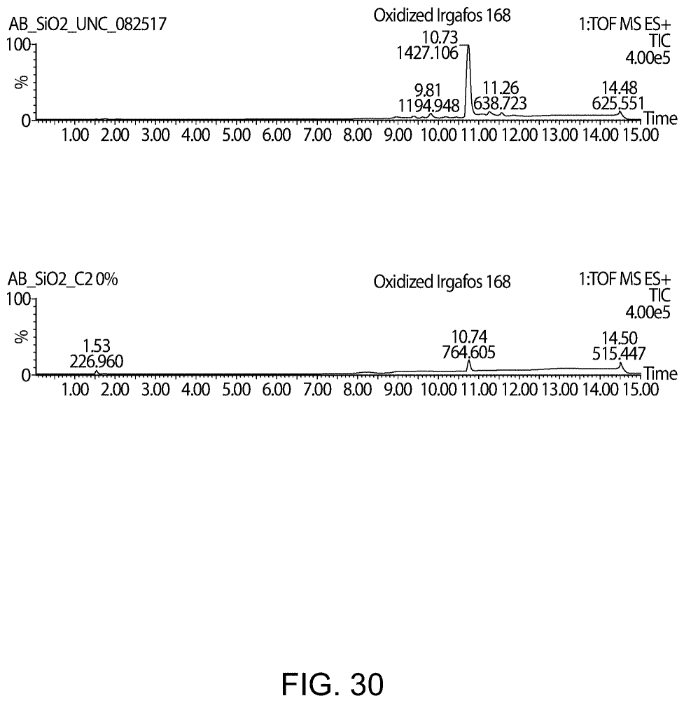

The results are presented in FIG. 30. The top scheme of FIG. 30 shows the peaks of extracted oxidized irgafos168 from uncoated film and the bottom scheme shows the peak of extracted oxidized irgafos168 from the protective layer coated film. The rest peaks are minimal. The results demonstrate that the protective coating effectively blocks the extractables from the film.

### Example 2

This example was to determine how much stretching/elongation that the pH protective coated film can tolerate with acceptable extractable-blocking function.

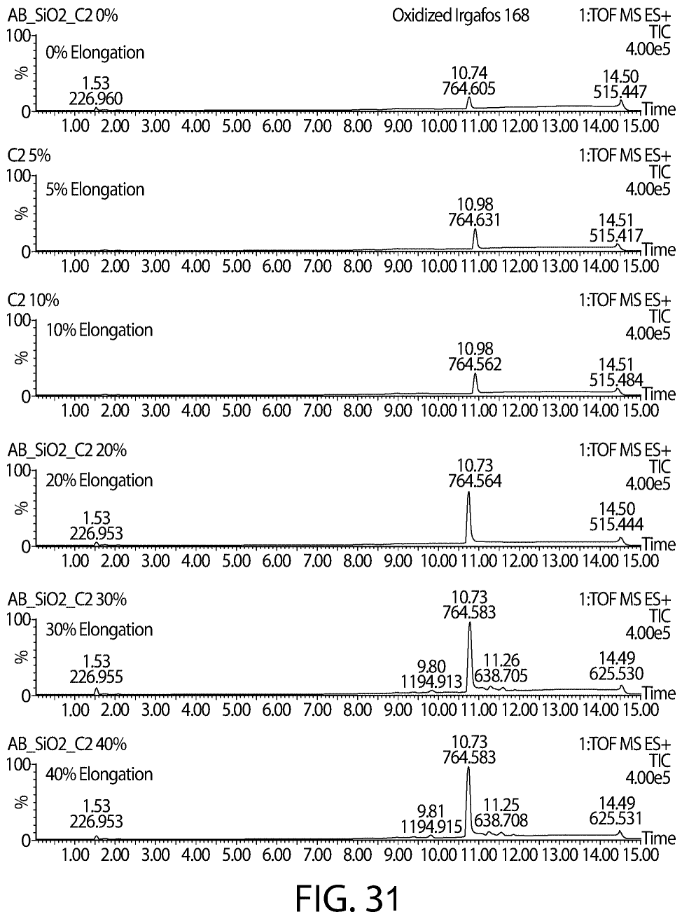

10×10 cm2 Square LLDPE film samples were coated according to the protective coating method described in the specification. After the completion of the coating process, the coated samples were subject to stretching/elongation condition. A Zwick electro-mechanical testing machine was used to stretch the films. The film samples were clamped in at a jaw spacing of 10 cm. Depending on the desired % stretch, samples were stretched up to 20 cm at a rate of 1 cm/s. In this example, the films were stretched by 5%, 10%, 20%, 30% and 40%. For each film after the desired stretching, a circle was “punched” out of the square film to fit snugly inside the PTFE lined lid of an i-chem glass sample jar (43.2 mm opening). The jar was filled with 3.0 ml of extraction fluid (EtOH). The i-chem lid was then secured onto the mouth of jar with the coated side of the film exposed to the inside of the jar. The surface area of the film in contact with the extraction fluid (EtOH) is 14.66 cm² (surface area/volume ratio of 4.9 cm²/ml). The jars were then placed in the incubation oven (50° C.) inverted so that the extraction fluid (EtOH) was in contact with the film. After the completion of the extraction, the extraction solution was analyzed using LC-MS spectroscopy. The results shown in FIG. 31 demonstrate that the peaks of extractables are still lower than that of the uncoated film after the coated film being stretched/elongated up to 20%.

### Example 3

This example was to visually assess the quality of the coating on the film surface after the coated film being stretched/elongated.

10×10 cm² Square LLDPE film samples were coated according to the pH protective coating method described in the specification. After the completion of the coating, the coated samples were subject to stretching condition. A Zwick electro-mechanical testing machine was used to stretch the films. The film samples were clamped in at a jaw spacing of 10 cm. Depending on desired % stretch, samples were stretched up to 20 cm at a rate of 1 cm/s. In this example, the films were stretched by 20%, 30% and 40%.

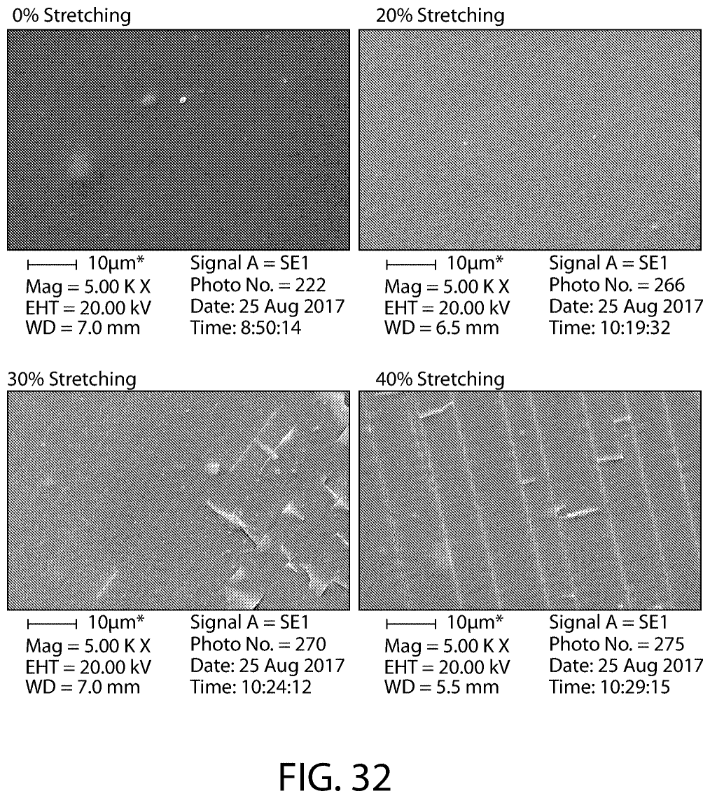

The stretched films were subject to SEM (Zeiss EVO 50 Scanning Electron Microscope) analysis to assess the coating quality after stretching experiment. The images are shown in FIG. 32. The images showed that the protective coating maintained intact by visual observation up to 20% of stretching.

### Example 4

This example was to evaluate the barrier coating of SiOx for its ability to maintain intactness under stretching/elongation conditions.

10×10 cm2 Square LLDPE film samples were coated with barrier coatings of SiOx according to the method described in the specification. After the completion of the coating, the coated samples were subject to stretching conditions as described in Example 4. The films were stretched by 5%, 10%, 50% and 100%.

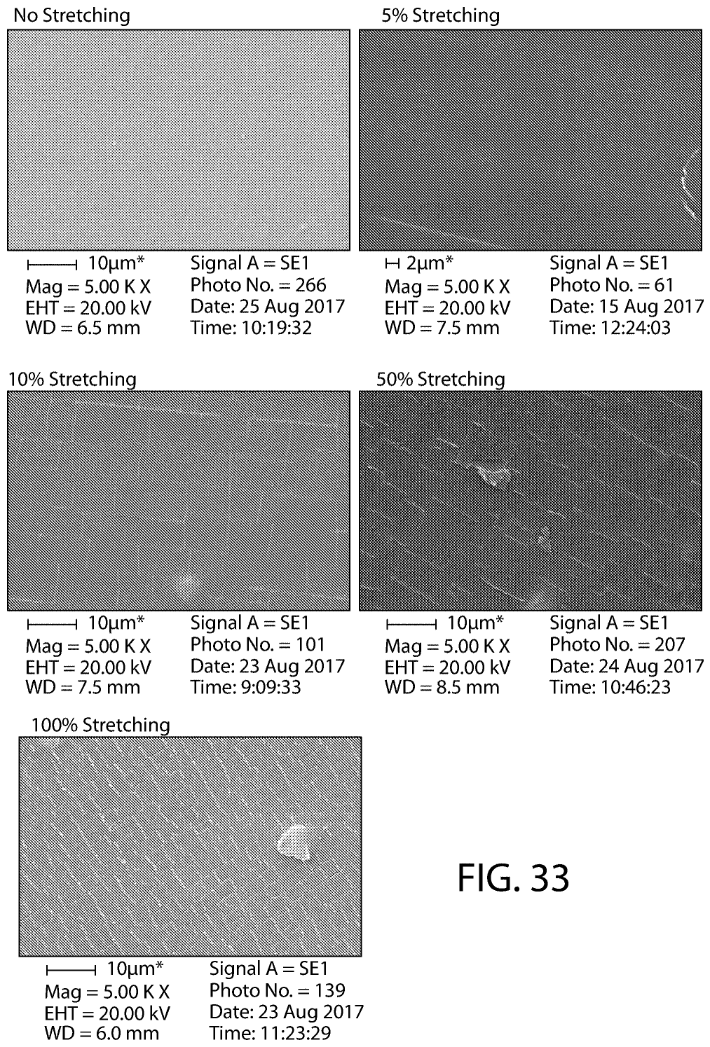

The stretched films were subject to SEM (Zeiss EVO 50 Scanning Electron Microscope) analysis to assess the coating quality after stretching. The images are presented in FIG. 33. The images show that the barrier coating of SiOx started cracking even at 5% of stretching while the pH protective coating in Example 4 maintains its intactness up to 20% of stretching/elongation. Comparing the performance of the barrier coating vs that of the pH protective coating under stretching/elongation conditions demonstrates that the pH protective coating of SiCxHy is advantageous in maintaining the coating intactness under stretching/elongation conditions.

### Example 5

The purpose of this example was to evaluate the extractable level of a trilayer coated film versus that of an uncoated film. In this experiment, the trilayer coated film was strectched/elongated to a different size.

The same uncoated film in Example 1 was used. The uncoated film was coated with trilayer coating according to the trilayer coating method described in the specification. The coating parameters are as follows.

### Trilayer Coating Parameters

| | Monomer | Oxygen | Power | Duration (s) |
|---|---|---|---|---|
| Adhesive | 10 | 0 | 250-350 | 30-60 |
| Barrier | 2-10 | 50-100 | 300-375 | 150-210 |
| Protective | 10 | 0 | 400-450 | 60-120 |

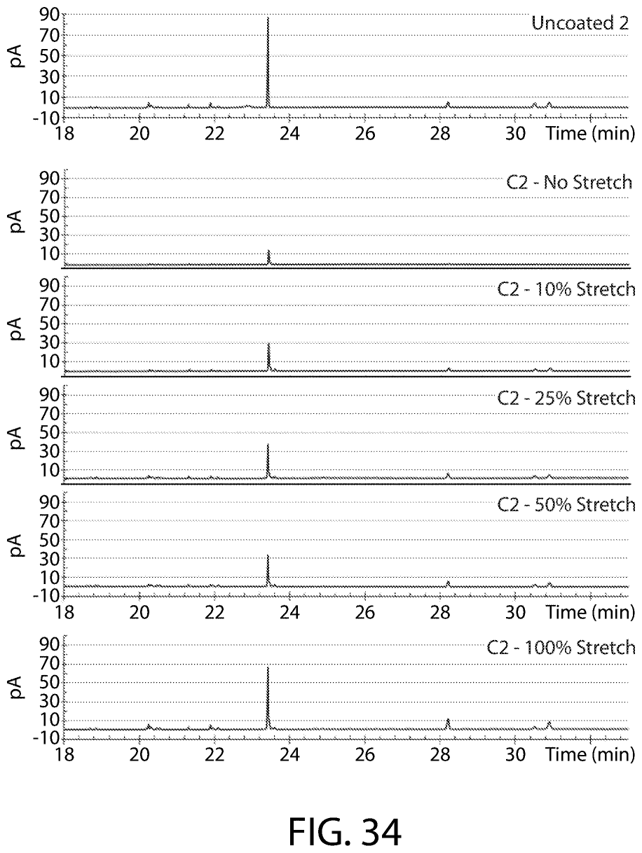

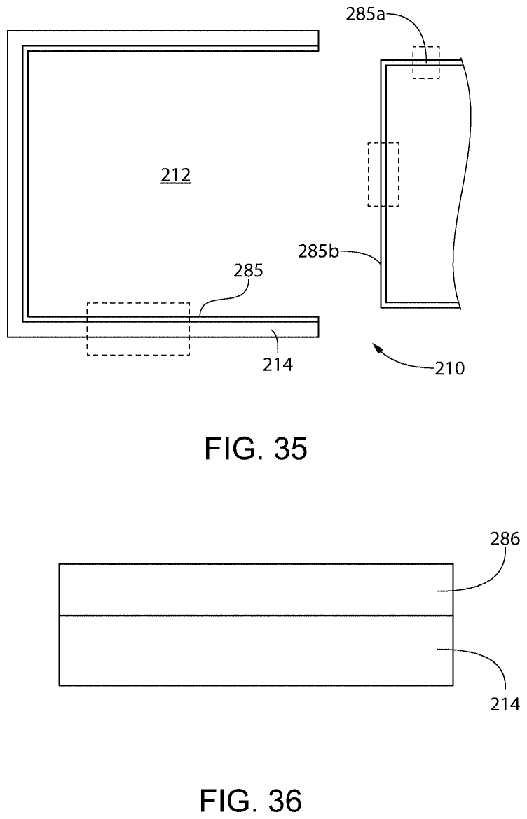

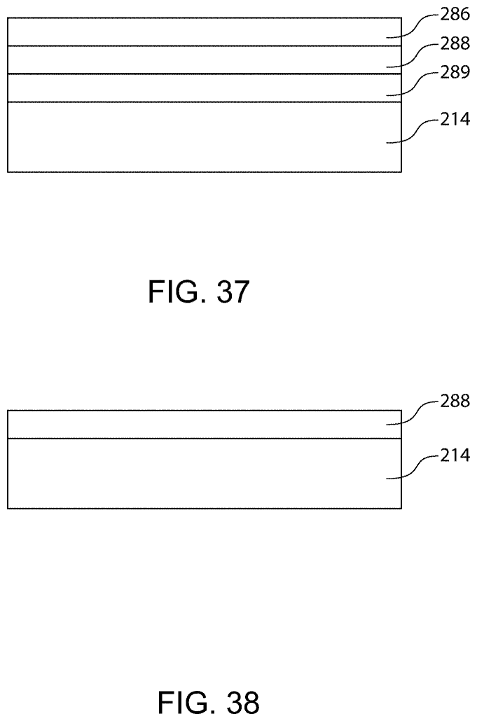

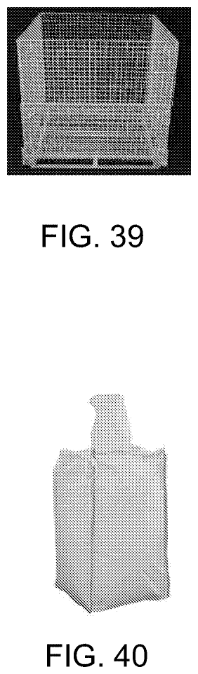

After the film was coated with trilayer coating, it was extracted in the same way as described in Example 1 except IPA (isopropyl alcohol) was used as the extraction solvent instead of EtOH. The extractables were evaluated by GC-FID. The results shown in FIG. 34 demonstrate that the trilayer coating is effective to block extractables even after stretching/elongation. The extractable peaks for trilayer coated film are still lower than uncoated film even after being stretched by 100% of the original size.

Non limiting examples of CAR-T related drug candidates or technologies for which the current disclosure can be used include:
Switchable CAR-T platform (AbbVie and Calibr)
UCART19 (Allogene)
Engineered autologous cell therapy (eACT™) platform (Amgen and Kite Pharma)
GoCAR-T technology (Bellicum Pharmaceuticals)
BB2121 (Bluebird Bio and Celgene)
Anti-GPC3 CAR-T for hepatocellular carcinoma (HCC), anti-GPC3 CAR-T for squamous lung cancer (SLC), cancer-specific anti-EGFR CAR-T for glioblastoma multiforme (GBM), and first-in-class anti-Claudin18.2-CAR-T for gastric and pancreatic cancer (CARsgen Therapeutics)
UCART19 and UCART123 (Cellectis)
T cell receptor (TCR) technology (Cell Medica)
Throttle™ and synNotch™ (Cell Design Labs)
NKR-T platform (Celyad and Dartmouth)
FT819 (Fate Therapeutics)
Yescarta (Gilead Sciences and Kite Pharma, approved by US FDA)
LCAR-B38M (Janssen Biotech)
CRISPR/Cas9-enhanced CAR-T therapies (Mustang Bio)
Kymriah (Novartis, approved by US FDA)
ARCUS genome editing technology (Precision Biosciences)
P-PSMA-101 (Poseida Therapeutics)
anti-CEA CAR-T (Sorrento Therapeutics)
non-viral “Sleeping Beauty” (SB) platform (Ziopharm)

While the disclosure has been illustrated and described in detail in the drawings and foregoing description, such illustration and description are to be considered illustrative or exemplary and not restrictive; the disclosure is not limited to the disclosed embodiments. Other variations to the disclosed embodiments can be understood and effected by those skilled in the art and practicing the claimed disclosure, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A pharmaceutical package or vessel capable of use in CAR-T cell therapy, comprising:
a polymeric wall comprising an interior surface and an outer surface;
a tie coating or layer of SiOₓC_{y}, wherein x is from about 0.5 to about 2.4 and y is from about 0.6 to about 3, on the interior surface of the polymeric wall; and/or
a barrier coating or layer of SiOₓ, wherein x is from 1.5 to 2.9, on the interior surface of the polymeric wall, or when present, the tie coating or layer of SiOₓC_{y}; and/or
a passivation coating or layer or pH protective coating or layer of SiOₓC_{y} or SiNₓC_{y}, wherein x is from about 0.5 to about 2.4 and y is from about 0.6 to about 3, on the interior surface of the polymeric wall or, when present, on the innermost surface of the tie coating or layer or the barrier coating or layer; and/or optionally
a surface layer or coating of any of, or combination of, the following:
silicon-based barrier coating system;
amorphous carbon coating;
fluorocarbon coating;
direct fluorination;
anti scratch/anti static coating;
antistatic coating;
antistatic additive compound in polymer;
oxygen scavenging additive compound in polymer;
colorant additive compound in polymer;
or antioxidation additive compound in polymer,
on any of the interior surface of the polymeric wall or, when present, the inner surface of any of the other coatings or layers.

2. The pharmaceutical package or vessel of claim 1, wherein the package or vessel is flexible or stretchable.

3. The pharmaceutical package or vessel of claim 1, wherein the package or vessel is a bag, a bioprocess bag, a transfer bag, single use bioreactor bag, a tube, a stopper, a connector or sleeve, a rigid frame, a flexible intermediate bulk container (FIBC), or on a pallet.

4. The pharmaceutical package or vessel of claim 1, wherein the polymeric wall is formed into the vessel or package by a laser welding device before or after the polymeric wall have been coated with the tie coating or layer and/or the barrier coating or layer and/or the passivation coating or layer or pH protective coating or layer and/or the surface layer or coating.

5. (canceled)

6. The pharmaceutical package or vessel of claim 4, wherein the laser welding uses a laser beam to melt the wall in a joint area of the parts of the walls to be joined by delivering a controlled amount of energy to a precise location and wherein a heat input of the laser beam is controlled by adjusting a laser beam size and/or moving the laser beam.

7. (canceled)

8. The pharmaceutical package or vessel of claim 6, wherein the laser beam is delivered to the joint area through the upper “transparent” part of the wall to be joined and is absorbed by the lower absorbing part, which converts infra-red (IR) energy into heat, and wherein the parts of the wall to be joined are held together by clamping for heat transfer between the parts.

9. (canceled)

10. The pharmaceutical package or vessel claim 1, further comprising carbon black and/or other absorbers blended into the resin of the polymeric wall.

11. The pharmaceutical package or vessel of claim 4, wherein the laser welding is facilitated by one or more micron-scale laser beams.

12. The pharmaceutical package or vessel of claim 4, wherein the laser welding device comprises:
fiber-optic cable;
scan head with mirrors coated for appropriate wave length;
focusing optics;
programmable multi-axis servo stages for accurate and reproducible laser beam delivery; and
one or more servo motors to move and precisely position the laser beam.

13. (canceled)

14. (canceled)

15. The pharmaceutical package or vessel of claim 2, wherein the coating(s) is able to maintain its desirable characteristics during stretching/elongation conditions.

16. The pharmaceutical package or vessel of claim 1, wherein the package or vessel contains a rigid structure, and wherein the rigid structure is a rigid support structure, a frame, a rigid box or a rigid container.

17. (canceled)

18. The pharmaceutical package or vessel of claim 15, wherein the layer(s) or coating(s) and the surface thereunder are being stretched/elongated by 5%, optionally 10%, optionally 20%, optionally 30%, optionally 40%, optionally 50%, optionally 70%, optionally 90%, optionally 100%, optionally 150%, optionally 200% of the original size.

19. The pharmaceutical package or vessel of claim 15, wherein the layer(s) or coating(s) affords improved barrier properties to gases, moisture and solvents and maintains the blocking properties after being stretched/elongated.

20. (canceled)

21. The pharmaceutical package or vessel of claim 2, wherein the layer(s) or coating(s) is effective to block extractables/leachables from the substrate and any coatings thereon and maintain the blocking properties after being stretched/elongated.

22. (canceled)

23. The pharmaceutical package or vessel of claim 1, wherein the polymeric wall comprises a film material selected from the group consisting of a polyolefin, a cyclic olefin polymer, a cyclic olefin copolymer, a polypropylene, a polyester, a polyethylene terephthalate (PET, PETE, PETP, or PET-P PET), a polyethylene naphthalate, a polycarbonate, a polylactic acid, an ethylene vinyl acetate (EVA), an ultra low density polyethylene (ULDPE), a linear low density polyethylene (LLDPE), a polyethylene vinyl alcohol-copolymers (EVOH), an Ethylene-vinyl acetate (EVA) material, a polyamide (PA) polymer, a synthetic polymer (such as polyamide or Nylon), an aliphatic polyamide, a semi-aromatic polyamide, a styrenic polymer or co polymer, or any combination, composite or blend of any two or more thereof.

24. (canceled)

25. A pharmaceutical package or vessel capable of use in CAR-T cell therapy including CAR-T cell manufacturing or treatment comprising:
a polymeric wall comprising an interior surface and an outer surface;
a tie coating or layer of SiOₓC_{y}, wherein x is from about 0.5 to about 2.4 and y is from about 0.6 to about 3, on the interior surface of the polymeric wall;
a barrier coating or layer of SiOₓ, wherein x is from 1.5 to 2.9, on an innermost surface of the tie coating or layer of SiOₓC_{y}; and
a passivation coating or layer or pH protective coating or layer of SiOₓC_{y} or SiNₓC_{y}, wherein x is from about 0.5 to about 2.4 and y is from about 0.6 to about 3, on an innermost surface of the barrier coating or layer.

26-40. (canceled)

41. The pharmaceutical package or vessel of claim 1, further comprising a pressure device, wherein the pressure device is selected from one or more of a pressure monitor, a pressure relief valve or a check valve, wherein the pressure monitor is capable of monitoring the pressure from 0 to about 1 psi.

42-44. (canceled)

45. The pharmaceutical package or vessel of claim 41, wherein the pressure monitor is compatible with gamma sterilization.

46. (canceled)

47. The pharmaceutical package or vessel of claim 41, which further comprises at least one port, and
wherein the pressure device is installed in one of the ports.

48. (canceled)

49. The pharmaceutical package or vessel of claim 1, wherein during multiple freezing/thawing processes:
the coating(s) is able to maintain its desirable characteristics and any pharmaceutical material contained in the package or vessel is able to maintain its integrity.

50. (canceled)
